(19) United States
(12) Patent Application Publication
(10) Pub. No.: US 2013/0184273 A1
(43) Pub. Date: Jul. 18, 2013

(54) BICYCLIC PYRIMIDINES

(71) Applicants: Stuart Ince, Berlin (DE); Hartmut Rehwinkel, Berlin (DE); Andrea Hägebarth, Berlin (DE); Oliver Politz, Panketal OT Zepernick (DE); Roland Neuhaus, Berlin (DE); Ulf Bömer, Glienicke (DE)

(72) Inventors: Stuart Ince, Berlin (DE); Hartmut Rehwinkel, Berlin (DE); Andrea Hägebarth, Berlin (DE); Oliver Politz, Panketal OT Zepernick (DE); Roland Neuhaus, Berlin (DE); Ulf Bömer, Glienicke (DE)

(73) Assignee: Bayer Intellectual Property GmbH, Monheim (DE)

(21) Appl. No.: 13/808,461

(22) PCT Filed: Jul. 11, 2011
(86) PCT No.: PCT/EP2011/061736

§ 371 Date: Mar. 27, 2013

(30) Foreign Application Priority Data
Jul. 13, 2010 (EP) 10169369.5

(51) Int. Cl. A61K 31/5377; A61K 31/519; A61K 45/06; C07D 487/04

(52) U.S. Cl. CPC A61K 31/5377; C07D 487/04; A61K 31/519; A61K 45/06
USPC 514/233.2; 544/263; 514/259.31; 544/281; 514/259.1; 544/117

(57) ABSTRACT

Compounds of formula (I), which are effective inhibitors of the Pi3K/Akt pathway, processes for their production and their use as pharmaceuticals.

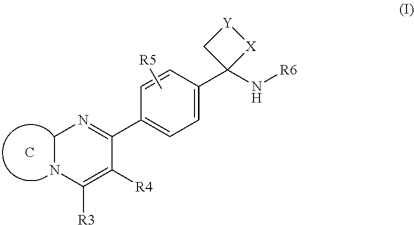

### FIELD OF APPLICATION OF THE INVENTION

The invention relates to substituted bicyclic pyrimidines, a process for their production and the use thereof.

### KNOWN TECHNICAL BACKGROUND

Cancer is the second most prevalent cause of death in the United States, causing 450,000 deaths per year. While substantial progress has been made in identifying some of the likely environmental and hereditary causes of cancer, there is a need for additional therapeutic modalities that target cancer and related diseases. In particular there is a need for therapeutic methods for treating diseases associated with dysregulated growth/proliferation.

Cancer is a complex disease arising after a selection process for cells with acquired functional capabilities like enhanced survival/resistance towards apoptosis and a limitless proliferative potential. Thus, it is preferred to develop drugs for cancer therapy addressing distinct features of established tumors.

One pathway that has been shown to mediate important survival signals for mammalian cells comprises receptor tyrosine kinases like platelet-derived growth factor receptor (PDGF-R), human epidermal growth factor 2/3 receptor (HER2/3), or the insulin-like growth factor 1 receptor (IGF-1R). After activation the respectives by ligand, these receptors activate the phoshatidylinositol 3-kinase (Pi3K)/Akt pathway. The phoshatidylinositol 3-kinase (Pi3K)/Akt protein kinase pathway is central to the control of cell growth, proliferation and survival, driving progression of tumors. Therefore within the class of serine-threonine specific signalling kinases, Akt (protein kinase B; PKB) with the isoenzmyes Akt1 (PKBα), Akt2 (PKB 11) and Akt3 (PKB γ) is of high interest for therapeutic intervention. Akt is mainly activated in a Pi3-kinase dependent manner and the activation is regulated through the tumor suppressor PTEN (phosphatase and tensin homolog), which works essentially as the functional antagonist of Pi3K.

The Pi3K/Akt pathway regulates fundamental cellular functions (e.g. transcription, translation, growth and survival), and is implicated in human diseases including diabetes and cancer. The pathway is frequently overactivated in a wide range of tumor entities like breast and prostate carcinomas. Upregulation can be due to overexpression or constitutively activation of receptor tyrosine kinases (e.g. EGFR, HER2/3), which are upstream and involved in its direct activation, or gain- or loss-of-function mutants of some of the components like loss of PTEN. The pathway is targeted by genomic alterations including mutation, amplification and rearrangement more frequently than any other pathway in human cancer, with the possible exception of the p53 and retinoblastoma pathways. The alterations of the Pi3K/Akt pathway trigger a cascade of biological events, that drive tumor progression, survival, angiogenesis and metastasis.

Activation of Akt kinases promotes increased nutrient uptake, converting cells to a glucose-dependent metabolism that redirects lipid precursors and amino acids to anabolic processes that support cell growth and proliferation. These metabolic phenotype with overactivated Akt lead to malignancies that display a metabolic conversion to aerobic glycolysis (the Warburg effect). In that respect the Pi3K/Akt pathway is discussed to be central for survival despite unfavourable growth conditions such as glucose depletion or hypoxia.

A further aspect of the activated PI3K/Akt pathway is to protect cells from programmed cell death (“apoptosis”) and is hence considered to transduce a survival signal. By acting as a modulator of anti-apoptotic signalling in tumor cells, the Pi3K/Akt pathway, particular Akt itself is a target for cancer therapy. Activated Akt phosphorylates and regulates several targets, e.g. BAD, GSK3 or FKHRL1, that affect different signalling pathways like cell survival, protein synthesis or cell movement. This Pi3K/Akt pathway also plays a major part in resistance of tumor cells to conventional anti-cancer therapies. Blocking the Pi3K/Akt pathway could therefore simultaneously inhibit the proliferation of tumor cells (e.g. via the inhibition of the metabolic effect) and sensitize towards pro-apoptotic agents.

Akt inhibition selectively sensitized tumor cells to apoptotic stimuli like Trail, Camptothecin and Doxorubicin. Dependent on the genetic background/molecular apperations of tumors, Akt inhibitors might induce apoptotic cell death in monotherapy as well.

From WO 2008/070016 tricyclic Akt inhibitors are known which are alledged to be unspecific Akt kinase inhibitors. No data for any specific compounds are disclosed. Different Akt inhibitors are disclosed in WO 2009/021992, WO2010088177, WO2010114780. In a recent disclosure, Y. Li et al (Bioorg. Med. Chem. Lett. 2009, 19, 834-836 and cited references therein) detail the difficulty in finding optimal Akt inhibitors. The potential application of Akt inhibitors in multiple disease settings, such as for example, cancer, makes the provision of new, improved Akt inhibitors to those currently available highly desirable.

### DESCRIPTION OF THE INVENTION

A solution to the above problem is the provision of improved Akt inhibitors, whereby the current compounds have an improved pharmacokinetic profile. It has now been found that the new substituted bicyclic pyrimidines, which are described in detail below, are Akt inhibitors with an improved pharmacokinetic profile.

In accordance with a first aspect, the invention relates to compounds of formula (I)

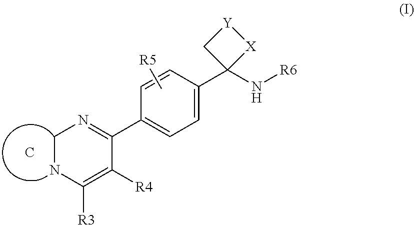

wherein ring C and the pyrimidine to which it is fused form a ring system selected from

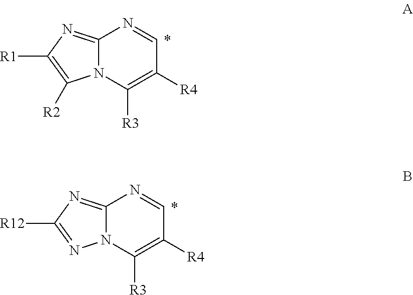

wherein * marks the point of the attachment,
wherein
R1 is hydrogen, or a group selected from 1-6C-alkyl, 3-7C-cycloalkyl, aryl, heteroaryl,
   wherein said group being optionally substituted, one or more times, identically or differently, with a substituent selected from:
   hydroxy, halogen, 1-6C-alkyl, 1-4C-haloalkyl, 1-6C-alkoxy, —NR8R9, cyano, —C(O)NR8R9, —C(O)OR10, —NHC(O)R11, —NHS(O)₂R11,
R12 is hydrogen, halogen, —NR13R14, or a group selected from 1-6C-alkyl, 3-7C-cycloalkyl, aryl, heteroaryl,
   wherein said group being optionally substituted, one or more times, identically or differently, with a substituent selected from:
   hydroxy, halogen, 1-6C-alkyl, 1-4C-haloalkyl, 1-6C-alkoxy, —NR8R9, cyano, —C(O)NR8R9, —C(O)OR10, —NHC(O)R11, —NHS(O)₂R11,
R2 is hydrogen, halogen, cyano, or a group selected from 1-6C-alkyl, 3-7C-cycloalkyl, aryl, heteroaryl, (1-6C-alkylen)-aryl, (1-6C-alkylen)-heteroaryl,
   wherein said group being optionally substituted, one or more times, identically or differently, with a substituent selected from:
   hydroxy, halogen, 1-6C-alkyl, 1-4C-haloalkyl, 1-6C-hydroxyalkyl, 1-6C-alkoxy, —NR8R9, cyano, —C(O)NR8R9, —C(O)OR10, —NHC(O)R11, —NHS(O)₂R11, —S(O)₂R11, —S(O)₂NR8R9,
R3 is hydrogen, 1-6C-alkyl, 3-7C-cycloalkyl, or NR15R16,
R4 is phenyl optionally substituted, one or more times, identically or differently, with a substituent selected from: 1-6C-alkyl, halogen, cyano,
R5 is hydrogen, halogen,
R6 is hydrogen, 1-6C-alkyl,
R8, R9 which can be the same or different, is hydrogen, 1-4C-alkyl (optionally substituted in the same way or differently one or more times with halogen, hydroxy, mono- or di-(1-4C-alkylamino), 1-4C-alkoxy); or 3-7C-cycloalkyl,
   or,
   in the case of —NR8R9, R8 and R9 together with the nitrogen to which they are attached may also form a 3-6C-heterocyclic ring,
R10 is hydrogen, 1-6C-alkyl,
R11 is 1-4C-alkyl (optionally substituted in the same way of differently one or more times with halogen, hydroxy) or 3-7C-cycloalkyl,
R13, R14 which can be the same or different, is hydrogen, or a group selected from 1-6C-alkyl, 3-7C-cycloalkyl, aryl, heteroaryl, (1-6C-alkylen)-aryl, (1-6C-alkylen)-heteroaryl,
   wherein said group being optionally substituted, one or more times, identically or differently, with a substituent selected from:
   hydroxy, halogen, 1-6C-alkyl, 1-4C-haloalkyl, 1-6C-alkoxy, cyano,
R15, R16 which can be the same or different, is hydrogen, or a group selected from 1-6C-alkyl, 3-7C-cycloalkyl, (1-6C-alkylen)-aryl, (1-6C-alkylen)-heteroaryl,
   wherein said group being optionally substituted, one or more times, identically or differently, with a substituent selected from:
   hydroxy, halogen, 1-6C-alkyl, 1-4C-haloalkyl, 1-6C-alkoxy, cyano, 3-7-cycloalkyl, heterocyclyl, —C(O)NR10R11, —C(O)OR10, —NHC(O)R11, —NHS(O)₂R11,
   or,
   R15 and R16 together with the nitrogen to which they are attached may also form a 3-6C-heterocyclic ring,
X is —(CH₂)ₙ—,
n is 0, 1, 2, or 3,
Y is —CH₂—, —CH(OH)—,
   or an N-oxide, a salt, a tautomer or a stereoisomer of said compound, or a salt of said N-oxide, tautomer or stereoisomer.

Another embodiment of the invention are compounds according to claim 1, wherein R1 is hydrogen, or a group selected from 1-6C-alkyl, 3-7C-cycloalkyl, aryl, heteroaryl,
wherein said group being optionally substituted, one or more times, identically or differently, with a substituent selected from:
   hydroxy, halogen, 1-6C-alkyl, 1-4C-haloalkyl, 1-6C-alkoxy, —NR8R9, cyano, —C(O)NR8R9, —C(O)OR10, —NHC(O)R11, —NHS(O)₂R11,
      R12 is hydrogen, halogen, —NR13R14, or a group selected from 1-6C-alkyl, 3-7C-cycloalkyl, aryl, heteroaryl,
wherein said group being optionally substituted, one or more times, identically or differently, with a substituent selected from:
hydroxy, halogen, 1-6C-alkyl, 1-4C-haloalkyl, 1-6C-alkoxy, —NR8R9, cyano, —C(O)NR8R9, —C(O)OR10, —NHC(O)R11, —NHS(O)₂R11,
   R2 is hydrogen, halogen, cyano, or a group selected from 1-6C-alkyl, 3-7C-cycloalkyl, aryl, heteroaryl, (1-6C-alkyl)-aryl, (1-6C-alkyl)-heteroaryl,
wherein said group being optionally substituted, one or more times, identically or differently, with a substituent selected from:
hydroxy, halogen, 1-6C-alkyl, 1-4C-haloalkyl, 1-6C-alkoxy, —NR8R9, cyano, —C(O)NR8R9, —C(O)OR10, —NHC(O)R11, —NHS(O)₂R11, —S(O)₂R11, —S(O)₂NR8R9,
   R3 is hydrogen, 1-6C-alkyl, 3-7C-cycloalkyl, or NR15R16,
   R4 is phenyl optionally substituted, one or more times, identically or differently, with a substituent selected from: 1-6C-alkyl, halogen, cyano,
   R5 is hydrogen, halogen,
   R6 is hydrogen, 1-6C-alkyl,
   R8, R9 which can be the same or different, is hydrogen, 1-4C-alkyl (optionally substituted in the same way of differently one or more times with halogen, hydroxy, mono- or di-(1-4C-alkylamino), 1-4C-alkoxy, or 3-7C-cycloalkyl,
or,
   in the case of —NR8R9, R8 and R9 together with the nitrogen to which they are attached may also form a 3-6C-heterocyclic ring,
   R10 is hydrogen, 1-6C-alkyl,
   R11 is 1-4C-alkyl (optionally substituted in the same way of differently one or more times with halogen, hydroxy) or 3-7C-cycloalkyl,
   R13, R14 which can be the same or different, is hydrogen, or a group selected from 1-6C-alkyl, 3-7C-cycloalkyl, aryl, heteroaryl, (1-6C-alkyl)-aryl, (1-6C-alkyl)-heteroaryl,
   wherein said group being optionally substituted, one or more times, identically or differently, with a substituent selected from:
   hydroxy, halogen, 1-6C-alkyl, 1-4C-haloalkyl, 1-6C-alkoxy, cyano,
   R15, R16 which can be the same or different, is hydrogen, or a group selected from 1-6C-alkyl, 3-7C-cycloalkyl, (1-6C-alkyl)-aryl, (1-6C-alkyl)-heteroaryl,
   wherein said group being optionally substituted, one or more times, identically or differently, with a substituent selected from: hydroxy, halogen, 1-6C-alkyl, 1-4C-haloalkyl, 1-6C-alkoxy, cyano, 3-7-cycloalkyl, heterocyclyl, —C(O)NR10R11, —C(O)OR10, —NHC(O)R11, —NHS(O)₂R11,
   or,
   in the case of —NR15R16, R15 and R16 together with the nitrogen to which they are attached may also form a 3-6C-heterocyclic ring,
   X is —(CH₂)ₙ—,
   n is 0, 1, 2, or 3,

### Y is —CH₂—, —CH(OH)—,

or an N-oxide, a salt, a tautomer or a stereoisomer of said compound, or a salt of said N-oxide, tautomer or stereoisomer.

A further embodiment of the invention are compounds according to claim 1, wherein

R1 is hydrogen, or a group selected from 1-6C-alkyl, 3-7C-cycloalkyl, aryl, heteroaryl,
wherein said group being optionally substituted, one or more times, identically or differently, with a substituent selected from:
hydroxy, halogen, 1-6C-alkyl, 1-4C-haloalkyl, 1-6C-alkoxy, —NR8R9, cyano, —C(O)NR8R9, —C(O)OR10, —NHC(O)R11, —NHS(O)₂R11,
R12 is hydrogen, halogen, —NR13R14, or a group selected from 1-6C-alkyl, 3-7C-cycloalkyl, aryl, heteroaryl,
wherein said group being optionally substituted, one or more times, identically or differently, with a substituent selected from:
hydroxy, halogen, 1-6C-alkyl, 1-4C-haloalkyl, 1-6C-alkoxy, —NR8R9, cyano, —C(O)NR8R9, —C(O)OR10, —NHC(O)R11, —NHS(O)₂R11,
R2 is hydrogen, halogen, cyano, or a group selected from 1-6C-alkyl, 3-7C-cycloalkyl, aryl, heteroaryl, (1-6C-alkylen)-aryl, (1-6C-alkylen)-heteroaryl,
wherein said group being optionally substituted, one or more times, identically or differently, with a substituent selected from:
hydroxy, halogen, 1-6C-alkyl, 1-4C-haloalkyl, 1-6C-hydroxyalkyl, 1-6C-alkoxy, —NR8R9, cyano, —C(O)NR8R9, —C(O)OR10, —NHC(O)R11, —NHS(O)₂R11, —S(O)₂R11, —S(O)₂NR8R9,
R3 is hydrogen, 1-6C-alkyl, 3-7C-cycloalkyl, or NR15R16,
R4 is phenyl optionally substituted, one or more times, identically or differently, with a substituent selected from: 1-6C-alkyl, halogen, cyano,
R5 is hydrogen, halogen,
R6 is hydrogen,
R8, R9 which can be the same or different, is hydrogen, 1-4C-alkyl (optionally substituted in the same way of differently one or more times with halogen, hydroxy, mono- or di-1-4C-alkylamino), 1-4C-alkoxy); or 3-7C-cycloalkyl,
or,
in the case of —NR8R9, R8 and R9 together with the nitrogen to which they are attached may also form a 3-6C-heterocyclic ring,
   R10 is hydrogen, 1-6C-alkyl,
   R11 is 1-4C-alkyl (optionally substituted in the same way of differently one or more times with halogen, hydroxy) or 3-7C-cycloalkyl,
   R13, R14 which can be the same or different, is hydrogen, or a group selected from 1-6C-alkyl, 3-7C-cycloalkyl, aryl, heteroaryl, (1-6C-alkylen)-aryl, (1-6C-alkylen)-heteroaryl,
   wherein said group being optionally substituted, one or more times, identically or differently, with a substituent selected from:
   hydroxy, halogen, 1-6C-alkyl, 1-4C-haloalkyl, 1-6C-alkoxy, cyano,
   R15, R16 which can be the same or different, is hydrogen, or a group selected from 1-6C-alkyl, 3-7C-cycloalkyl, (1-6C-alkylen)-aryl, (1-6C-alkylen)-heteroaryl,
   wherein said group being optionally substituted, one or more times, identically or differently, with a substituent selected from:
   hydroxy, halogen, 1-6C-alkyl, 1-4C-haloalkyl, 1-6C-alkoxy, cyano, 3-7-cycloalkyl, heterocyclyl, —C(O)OR10, or
   R15, R16 together with the nitrogen atom to which they are attached may also form a 5- or 6 membered heterocyclic ring optionally containing an additional nitrogen- or oxygen atom,
   X is —(CH₂)ₙ—,
   n is 0, 1, 2, or 3,

### Y is —CH₂—, —CH(OH)—,

or an N-oxide, a salt, a tautomer or a stereoisomer of said compound, or a salt of said N-oxide, tautomer or stereoisomer. or an N-oxide, a salt, a tautomer or a stereoisomer of said compound, or a salt of said N-oxide, tautomer or stereoisomer.

Another aspect of the invention are compounds of formula (I)

wherein
R1 is hydrogen, or a group selected from 1-6C-alkyl, 3-7C-cycloalkyl, aryl, heteroaryl,
   wherein said group being optionally substituted, one or more times, identically or differently, with a substituent selected from:
   hydroxy, halogen, 1-6C-alkyl, 1-4C-haloalkyl, 1-6C-alkoxy, —NR8R9, cyano, —C(O)NR8R9, —C(O)OR10, —NHC(O)R11, —NHS(O)₂R11,
R12 is hydrogen, halogen, —NR13R14, or a group selected from 1-6C-alkyl, 3-7C-cycloalkyl, aryl, heteroaryl,
   wherein said group being optionally substituted, one or more times, identically or differently, with a substituent selected from:
   hydroxy, halogen, 1-6C-alkyl, 1-4C-haloalkyl, 1-6C-alkoxy, —NR8R9, cyano, —C(O)NR8R9, —C(O)OR10, —NHC(O)R11, —NHS(O)₂R11,
R2 is hydrogen, halogen, cyano, or a group selected from 1-6C-alkyl, 3-7C-cycloalkyl, aryl, heteroaryl, (1-6C-alkyl)-aryl, (1-6C-alkyl)-heteroaryl,
   wherein said group being optionally substituted, one or more times, identically or differently, with a substituent selected from:
   hydroxy, halogen, 1-6C-alkyl, 1-4C-haloalkyl, 1-6C-alkoxy, —NR8R9, cyano, —C(O)NR8R9, —C(O)OR10, —NHC(O)R11, —NHS(O)₂R11, —S(O)₂R11, —S(O)₂NR8R9,
R3 is hydrogen, 1-6C-alkyl, 3-7C-cycloalkyl, or NR15R16,
R4 is phenyl optionally substituted, one or more times, identically or differently, with a substituent selected from: 1-6C-alkyl, halogen, cyano,
R5 is hydrogen, halogen,
R6 is hydrogen,
R8, R9 which can be the same or different, is hydrogen, 1-4C-alkyl (optionally substituted in the same way of differently one or more times with halogen, hydroxy, mono- or di-(1-4C-alkylamino), 1-4C-alkoxy, or 3-7C-cycloalkyl,
   or,
   in the case of —NR8R9, R8 and R9 together with the nitrogen to which they are attached may also form a 3-6C-heterocyclic ring,
R10 is hydrogen, 1-6C-alkyl,
R11 is 1-4C-alkyl (optionally substituted in the same way of differently one or more times with halogen, hydroxy) or 3-7C-cycloalkyl,
R13, R14 which can be the same or different, is hydrogen, or a group selected from 1-6C-alkyl, 3-7C-cycloalkyl, aryl, heteroaryl, (1-6C-alkyl)-aryl, (1-6C-alkyl)-heteroaryl,
   wherein said group being optionally substituted, one or more times, identically or differently, with a substituent selected from:
   hydroxy, halogen, 1-6C-alkyl, 1-4C-haloalkyl, 1-6C-alkoxy, cyano,
R15, R16 which can be the same or different, is hydrogen, or a group selected from 1-6C-alkyl, 3-7C-cycloalkyl, (1-6C-alkyl)-aryl, (1-6C-alkyl)-heteroaryl,
   wherein said group being optionally substituted, one or more times, identically or differently, with a substituent selected from:
   hydroxy, halogen, 1-6C-alkyl, 1-4C-haloalkyl, 1-6C-alkoxy, cyano, 3-7-cycloalkyl, heterocyclyl, —C(O)OR10,
X is —(CH₂)ₙ—,
n is 0, 1, 2, or 3,
Y is —CH₂—, —CH(OH)—,
   or an N-oxide, a salt, a tautomer or a stereoisomer of said compound, or a salt of said N-oxide, tautomer or stereoisomer. or an N-oxide, a salt, a tautomer or a stereoisomer of said compound, or a salt of said N-oxide, tautomer or stereoisomer.

A further embodiment of the invention are compounds according to claim 1, wherein

R1 is hydrogen, or a group selected from 1-6C-alkyl, 3-7C-cycloalkyl, aryl, heteroaryl,
wherein said group being optionally substituted, one or more times, identically or differently, with a substituent selected from:
hydroxy, halogen, 1-6C-alkyl, 1-4C-haloalkyl, 1-6C-alkoxy, —NR8R9, cyano, —C(O)NR8R9, —C(O)OR10, —NHC(O)R11, —NHS(O)₂R11,
R12 is hydrogen, halogen, —NR13R14, or a group selected from 1-6C-alkyl, 3-7C-cycloalkyl, aryl, heteroaryl,
wherein said group being optionally substituted, one or more times, identically or differently, with a substituent selected from:
hydroxy, halogen, 1-6C-alkyl, 1-4C-haloalkyl, 1-6C-alkoxy, —NR8R9, cyano, —C(O)NR8R9, —C(O)OR10, —NHC(O)R11, —NHS(O)₂R11,
R2 is hydrogen, halogen, cyano, or a group selected from 1-6C-alkyl, 3-7C-cycloalkyl, aryl, heteroaryl, (1-6C-alkylen)-aryl, (1-6C-alkylen)-heteroaryl,
wherein said group being optionally substituted, one or more times, identically or differently, with a substituent selected from:
hydroxy, halogen, 1-6C-alkyl, 1-4C-haloalkyl, 1-6C-hydroxyalkyl, 1-6C-alkoxy, —NR8R9, cyano, —C(O)NR8R9, —C(O)OR10, —NHC(O)R11, —NHS(O)₂R11, —S(O)₂R11, —S(O)₂NR8R9,
R3 is hydrogen, 1-6C-alkyl, 3-7C-cycloalkyl, or NR15R16,
R4 is phenyl,
R5 is hydrogen,
R6 is hydrogen,
R8, R9 which can be the same or different, is hydrogen, 1-4C-alkyl (optionally substituted in the same way or differently one or more times with halogen, hydroxy, mono- or di-(1-4C-alkylamino), 1-4C-alkoxy); or 3-7C-cycloalkyl,
or,
in the case of —NR8R9, R8 and R9 together with the nitrogen to which they are attached may also form a 3-6C-heterocyclic ring,
   R10 is hydrogen, 1-6C-alkyl,
   R11 is 1-4C-alkyl (optionally substituted in the same way of differently one or more times with halogen, hydroxy) or 3-7C-cycloalkyl,
   R13, R14 which can be the same or different, is hydrogen, or a group selected from 1-6C-alkyl, 3-7C-cycloalkyl, aryl, heteroaryl, (1-6C-alkylen)-aryl, (1-6C-alkylen)-heteroaryl,
   wherein said group being optionally substituted, one or more times, identically or differently, with a substituent selected from:
   hydroxy, halogen, 1-6C-alkyl, 1-4C-haloalkyl, 1-6C-alkoxy, cyano,
   R15, R16 which can be the same or different, is hydrogen, or a group selected from 1-6C-alkyl, 3-7C-cycloalkyl, (1-6C-alkyl)-aryl, (1-6C-alkyl)-heteroaryl,
   wherein said group being optionally substituted, one or more times, identically or differently, with a substituent selected from:
   hydroxy, halogen, 1-6C-alkyl, 1-4C-haloalkyl, 1-6C-alkoxy, cyano, 3-7-cycloalkyl, heterocyclyl, —C(O)OR10, or
   or R15, R16 together with the nitrogen atom to which they are attached may also form a 5- or 6-membered heterocyclic ring optionally containing an additional nitrogen- or oxygen atom,
   X is —(CH₂)ₙ—,
   n is 0, 1 or 2,

### Y is —CH₂—, —CH(OH)—,

or an N-oxide, a salt, a tautomer or a stereoisomer of said compound, or a salt of said N-oxide, tautomer or stereoisomer.

Another aspect of the invention are compounds of formula (I) wherein
R1 is hydrogen, or a group selected from 1-6C-alkyl, 3-7C-cycloalkyl, aryl, heteroaryl,
   wherein said group being optionally substituted, one or more times, identically or differently, with a substituent selected from:
   hydroxy, halogen, 1-6C-alkyl, 1-4C-haloalkyl, 1-6C-alkoxy, —NR8R9, cyano, —C(O)NR8R9, —C(O)OR10, —NHC(O)R11, —NHS(O)₂R11,
R12 is hydrogen, halogen, —NR13R14, or a group selected from 1-6C-alkyl, 3-7C-cycloalkyl, aryl, heteroaryl,
   wherein said group being optionally substituted, one or more times, identically or differently, with a substituent selected from:
   hydroxy, halogen, 1-6C-alkyl, 1-4C-haloalkyl, 1-6C-alkoxy, —NR8R9, cyano, —C(O)NR8R9, —C(O)OR10, —NHC(O)R11, —NHS(O)₂R11,
R2 is hydrogen, halogen, cyano, or a group selected from 1-6C-alkyl, 3-7C-cycloalkyl, aryl, heteroaryl, (1-6C-alkyl)-aryl, (1-6C-alkyl)-heteroaryl,
   wherein said group being optionally substituted, one or more times, identically or differently, with a substituent selected from:
   hydroxy, halogen, 1-6C-alkyl, 1-4C-haloalkyl, 1-6C-alkoxy, —NR8R9, cyano, —C(O)NR8R9, —C(O)OR10, —NHC(O)R11, —NHS(O)₂R11, —S(O)₂R11, —S(O)₂NR8R9,
R3 is hydrogen, 1-6C-alkyl, 3-7C-cycloalkyl, or NR15R16,
R4 is phenyl,
R5 is hydrogen,
R6 is hydrogen,
R8, R9 which can be the same or different, is hydrogen, 1-4C-alkyl (optionally substituted in the same way of differently one or more times with halogen, hydroxy, mono- or di-(1-4C-alkylamino), 1-4C-alkoxy, or 3-7C-cycloalkyl,
   or,
   in the case of —NR8R9, R8 and R9 together with the nitrogen to which they are attached may also form a 3-6C-heterocyclic ring,
R10 is hydrogen, 1-6C-alkyl,
R11 is 1-4C-alkyl (optionally substituted in the same way of differently one or more times with halogen, hydroxy) or 3-7C-cycloalkyl,
R13, R14 which can be the same or different, is hydrogen, or a group selected from 1-6C-alkyl, 3-7C-cycloalkyl, aryl, heteroaryl, (1-6C-alkyl)-aryl, (1-6C-alkyl)-heteroaryl,
   wherein said group being optionally substituted, one or more times, identically or differently, with a substituent selected from:
   hydroxy, halogen, 1-6C-alkyl, 1-4C-haloalkyl, 1-6C-alkoxy, cyano,
R15, R16 which can be the same or different, is hydrogen, or a group selected from 1-6C-alkyl, 3-7C-cycloalkyl, (1-6C-alkyl)-aryl, (1-6C-alkyl)-heteroaryl,
   wherein said group being optionally substituted, one or more times, identically or differently, with a substituent selected from:
   hydroxy, halogen, 1-6C-alkyl, 1-4C-haloalkyl, 1-6C-alkoxy, cyano, 3-7-cycloalkyl, heterocyclyl, —C(O)OR10,
X is —(CH₂)ₙ—,
n is 0, 1, 2, or 3,
Y is —CH₂—, —CH(OH)—,
or an N-oxide, a salt, a tautomer or a stereoisomer of said compound, or a salt of said N-oxide, tautomer or stereoisomer.

A further embodiment of the invention are compounds according to claim 1, wherein

R1 is hydrogen, or a group selected from 1-3C-alkyl, 3-6C-cycloalkyl, aryl, heteroaryl,
wherein said group being optionally substituted, one or more times, identically or differently, with a substituent selected from:
hydroxy, halogen, 1-3C-alkyl, 1-3C-haloalkyl, 1-3C-alkoxy, —NR8R9, cyano, —C(O)NR8R9, —C(O)OR10, —NHC(O)R11, —NHS(O)₂R11,
R12 is hydrogen, halogen, —NR13R14, or a group selected from 1-3C-alkyl, 3-6C-cycloalkyl, aryl, heteroaryl,
wherein said group being optionally substituted, one or more times, identically or differently, with a substituent selected from:
hydroxy, halogen, 1-3C-alkyl, 1-3C-haloalkyl, 1-3C-alkoxy, —NR8R9, cyano, —C(O)NR8R9, —C(O)OR10, —NHC(O)R11, —NHS(O)₂R11,
R2 is hydrogen, halogen, cyano, or a group selected from 1-3C-alkyl, 3-6C-cycloalkyl, aryl, heteroaryl, (1-3C-alkylen)-aryl, (1-3C-alkylen)-heteroaryl,
wherein said group being optionally substituted, one or more times, identically or differently, with a substituent selected from:
hydroxy, halogen, 1-3C-alkyl, 1-3C-haloalkyl, 1-3C-hydroxyalkyl, 1-3C-alkoxy, —NR8R9, cyano, —C(O)NR8R9, —C(O)OR10, —NHC(O)R11, —NHS(O)₂R11, —S(O)₂R11, —S(O)₂NR8R9,
R3 is hydrogen, 1-3C-alkyl, 3-6C-cycloalkyl, or NR15R16,
R4 is phenyl,
R5 is hydrogen,
R6 is hydrogen,
R8, R9 which can be the same or different, is hydrogen, 1-3C-alkyl (optionally substituted in the same way or differently one or more times with halogen, hydroxy, mono- or di-(1-3C-alkylamino), 1-3C-alkoxy); or 3-6C-cycloalkyl,
or,
in the case of —NR8R9, R8 and R9 together with the nitrogen to which they are attached may also form a 3-6C-heterocyclic ring,
   R10 is hydrogen, 1-3C-alkyl,
   R11 is 1-3C-alkyl (optionally substituted in the same way of differently one or more times with halogen, hydroxy) or 3-6C-cycloalkyl,
   R13, R14 which can be the same or different, is hydrogen, or a group selected from 1-3C-alkyl, 3-6C-cycloalkyl, aryl, heteroaryl, (1-3C-alkylen)-aryl, (1-3C-alkylen)-heteroaryl,
   wherein said group being optionally substituted, one or more times, identically or differently, with a substituent selected from:
   hydroxy, halogen, 1-3C-alkyl, 1-3C-haloalkyl, 1-3C-alkoxy, cyano,
   R15, R16 which can be the same or different, is hydrogen, or a group selected from 1-3C-alkyl, 3-6C-cycloalkyl, (1-3C-alkylen)-aryl, (1-3C-alkylen)-heteroaryl, wherein said group being optionally substituted, one or more times, identically or differently, with a substituent selected from:
   hydroxy, halogen, 1-3C-alkyl, 1-3C-haloalkyl, 1-3C-alkoxy, cyano, 3-6-cycloalkyl, heterocyclyl, —C(O)OR10, or
   or R15, R16 together with the nitrogen atom to which they are attached may also form a 5- or 6-membered heterocyclic ring optionally containing an additional nitrogen- or oxygen atom,
   X is —(CH₂)ₙ—,
   n is 0, 1 or 2,

### Y is —CH₂—, —CH(OH)—,

or an N-oxide, a salt, a tautomer or a stereoisomer of said compound, or a salt of said N-oxide, tautomer or stereoisomer.

A further embodiment of the invention are compounds according to claim 1,

wherein
R1 is hydrogen, 1-6C-alkyl,
R12 is hydrogen, 1-6C-alkyl, 3-7C-cycloalkyl, heteroaryl, NR13R14,
R2 is hydrogen, aryl, heteroaryl
wherein said aryl being optionally substituted, one or more times, identically or differently, with a substituent selected from:
halogen, 1-6C-hydroxyalkyl, cyano, —S(O)₂R11, C(O)NR8R9,
R3 is hydrogen, 1-6C-alkyl, NR15R16,
R4 is phenyl,
R5 is hydrogen,
R6 is hydrogen,
R8, R9 is hydrogen,
R10 is hydrogen, 1-4C-alkyl,

### R11 is 1-4C-alkyl,

R13, R14 which can be the same or different, is hydrogen, or a group selected from 1-6C-alkyl, aryl, (1-6C-alkylen)-heteroaryl, wherein said group is optionally substituted with 1-6C-alkoxy,
R15, R16 which can be the same or different, is hydrogen, or a group selected from 1-6C-alkyl, 3-7C-cycloalkyl, (1-6C-alkylen)-aryl,
wherein said group being optionally substituted, one or more times, identically or differently, with a substituent selected from:
hydroxy, halogen, 1-6C-alkoxy, 3-7C-cycloalkyl, —C(O)OR10, or R15, R16 together with the nitrogen atom to which they are attached may also form a 6-membered ring containing one oxygen atom,
   X is —(CH₂)ₙ—,
   n is 0, 1, or 2

### Y is —CH₂—,

or an N-oxide, a salt, a tautomer or a stereoisomer of said compound, or a salt of said N-oxide, tautomer or stereoisomer.

A further embodiment of the invention are compounds according to claim 1, wherein

R1 is hydrogen, 1-3C-alkyl,
R12 is hydrogen, 1-3C-alkyl, 3-6C-cycloalkyl, heteroaryl, NR13R14,
R2 is hydrogen, aryl, heteroaryl
wherein said aryl being optionally substituted, one or more times, identically or differently, with a substituent selected from:
halogen, 1-3C-hydroxyalkyl, cyano, —S(O)₂R11, C(O)NR8R9,
R3 is hydrogen, 1-3C-alkyl, NR15R16,
R4 is phenyl,
R5 is hydrogen,
R6 is hydrogen,
R8, R9 is hydrogen,
R10 is hydrogen, 1-3C-alkyl,

### R11 is 1-3C-alkyl,

R13, R14 which can be the same or different, is hydrogen, or a group selected from 1-3C-alkyl, aryl, (1-3C-alkylen)-heteroaryl, wherein said group is optionally substituted with 1-3C-alkoxy,
R15, R16 which can be the same or different, is hydrogen, or a group selected from 1-3C-alkyl, 3-6C-cycloalkyl, (1-3C-alkylen)-aryl,
wherein said group being optionally substituted, one or more times, identically or differently, with a substituent selected from:
hydroxy, halogen, 1-6C-alkoxy, 3-7C-cycloalkyl, —C(O)OR10, or R15, R16 together with the nitrogen atom to which they are attached may also form a 6-membered ring containing one oxygen atom,
   X is —(CH₂)ₙ—,
   n is 0, 1 or 2

### Y is —CH₂—,

or an N-oxide, a salt, a tautomer or a stereoisomer of said compound, or a salt of said N-oxide, tautomer or stereoisomer.

Another aspect of the invention are compounds of formula (I)

wherein,
R1 is hydrogen, 1-6C-alkyl,
R12 is hydrogen, 1-6C-alkyl, 3-7C-cycloalkyl, heteroaryl, NR13R14,
R2 is hydrogen, halogen, aryl,
   wherein said aryl being optionally substituted, one or more times, identically or differently, with a substituent selected from:
   halogen, cyano, —S(O)₂R11,
R3 is hydrogen, 1-6C-alkyl, NR15R16,
R4 is phenyl,
R5 is hydrogen
R6 is hydrogen,
R11 is 1-4C-alkyl,
R13, R14 which can be the same or different, is hydrogen, or a group selected from 1-6C-alkyl, aryl, (1-6C-alkyl)-heteroaryl wherein said group is optionally substituted with 1-6C-alkoxy,
R15, R16 which can be the same or different, is hydrogen, or a group selected from 1-6C-alkyl, 3-7C-cycloalkyl, (1-6C-alkyl)-aryl, (1-6C-alkyl)heteroaryl, wherein said group being optionally substituted, one or more times, identically or differently, with a substituent selected from:
halogen, 1-6C-alkyl, 1-6C-alkoxy, 3-7C-cycloalkyl, —C(O)OR10,
X is —(CH₂)ₙ—.
n is 0, 1, or 2,
Y is —CH₂—,
   or an N-oxide, a salt, a tautomer or a stereoisomer of said compound, or a salt of said N-oxide, tautomer or stereoisomer.

A further embodiment of the invention are compounds according to claim 1,

wherein
R1 is hydrogen, methyl,
R12 is hydrogen, methyl, cyclopropyl, N-methyl-pyrazolyl, pyridyl, NR13R14,
R2 is hydrogen, 1H-pyrazol-yl, or phenyl substituted one or more times with fluorine, cyano, —S(O)₂R11, C(O)NR8R9, hydroxymethyl,
R3 is hydrogen, methyl, NR15R16,
R4 is phenyl,
R5 is hydrogen,
R6 is hydrogen,
R8, R9 which is hydrogen,
R11 is methyl
R13, R14 which can be the same or different, is hydrogen, methyl, ethyl, —CH(CH3)2, —(CH₂)₂—OCH₃, phenyl, —CH₂— (pyridyl),
R15, R16 which can be the same or different, is hydrogen, cyclopropyl, cyclobutyl which are optionally substituted by —C(O)OCH₂CH₃, cyclohexyl optionally substituted by hydroxy, or 1-4C-alkyl optionally substituted with methoxy, cyclopropyl, 4-fluoro-phenyl, or
R15, R16 together with the nitrogen atom to which they are attached form a morpholine ring,
X is —(CH₂—)ₙ,
n is 0, 1 or 2

### Y is —CH₂—,

or an N-oxide, a salt, a tautomer or a stereoisomer of said compound, or a salt of said N-oxide, tautomer or stereoisomer.

Another aspect of the invention are compounds of formula (I)

wherein,
R1 is hydrogen, methyl,
R12 is hydrogen, methyl, cyclopropyl, N-methyl-pyrazole, pyridine, NR13R14,
R2 is hydrogen, bromine, or, phenyl substituted with cyano, —S(O)₂R11,
R3 is hydrogen, methyl, NR15R16,
R4 is phenyl,
R5 is hydrogen,
R6 is hydrogen,
R11 is methyl
R13, R14 which can be the same or different, is hydrogen, methyl, ethyl, —(CH₂)₂—OCH₃, phenyl, —CH₂-(pyridyl),
R15, R16 which can be the same or different, is hydrogen, methyl, isopropyl, cyclopropyl, cyclobutyl optionally substituted by —C(O)OCH₂CH₃, or 1-4C-alkyl substituted with methoxy, cyclopropyl, 4-fluoro-phenyl, N-methyl-pyrazole,
X is —(CH₂)ₙ—,
n is 0, 1, or 2,
Y is —CH₂—,
   or an N-oxide, a salt, a tautomer or a stereoisomer of said compound, or a salt of said N-oxide, tautomer or stereoisomer.
   In one aspect of the invention compounds of formula (I) as described above are selected from the group consisting of
1-[4-(2-methyl-6-phenyl-[1,2,4]triazolo[1,5-a]pyrimidin-5-yl)phenyl]-cyclobutylamine,
1-[4-(2-methyl-6-phenyl-[1,2,4]triazolo[1,5-a]pyrimidin-5-yl)phenyl]-cyclopentylamine,
1-[4-(2-methyl-6-phenyl-[1,2,4]triazolo[1,5-a]pyrimidin-5-yl)phenyl]-cyclopropylamine,
1-[4-(2,7-dimethyl-6-phenyl-[1,2,4]triazolo[1,5-a]pyrimidin-5-yl)phenyl]-cyclobutylamine,
1-[4-(2-cyclopropyl-6-phenyl-[1,2,4]triazolo[1,5-a]pyrimidin-5-yl)-phenyl]-cyclobutylamine,
1-[4-(6-Phenyl-imidazo[1,2-a]pyrimidin-7-yl)-phenyl]-cyclobutylamine,
1-[4-(2-Methyl-6-phenyl-imidazo[1,2-a]pyrimidin-7-yl)-phenyl]-cyclobutylamine,
{7-[4-(1-Amino-cyclobutyl)-phenyl]-6-phenyl-imidazo[1,2-a]pyrimidin-5-yl)-cyclopropylamine,
1-[4-(5-Morpholino-4-yl-6-phenyl-imidazo[1,2-a]pyrimidin-7-yl)-phenyl]-cyclobutylamine,
{7-[4-(1-Amino-cyclobutyl)-phenyl]-6-phenyl-imidazo[1,2-a]pyrimidin-5-yl}-((S)-2-methoxy-1-methyl-ethyl)-amine,
{7-[4-(1-Amino-cyclobutyl)-phenyl]-2-methyl-6-phenyl-imidazo[1,2-a]pyrimidin-5-yl)-cyclopropylamine,
{7-[4-(1-Amino-cyclobutyl)-phenyl]-2-methyl-6-phenyl-imidazo[1,2-a]pyrimidin-5-yl}-((S)-2-methoxy-1-methyl-ethyl)-amine,
{5-[4-(1-Aminocyclobutyl)-phenyl]-6-phenyl-2-pyridine-2-yl-[1,2,4]triazolo[1,5-a]pyrimidin-7-yl}-tert.-butylamine,
{5-[4-(1-Aminocyclobutyl)-phenyl]-6-phenyl-2-pyridine-2-yl-[1,2,4]triazolo[1,5-a]pyrimidin-7-yl}-cyclopropylamine,
1-[4-(7-Morpholine-4-yl-6-phenyl-2-pyridine-2-yl-[1,2,4]triazolo[1,5-a]pyrimidin-5-yl)phenyl]-cyclobutylamine,
1-{5-[4-(1-Aminocyclobutyl)-phenyl]-2-methyl-6-phenyl-[1,2,4]triazolo[1,5-a]pyrimidin-7-ylamino}cyclobutanecarboxylic acid ethyl ester,
{5-[4-(1-Amino-cyclobutyl)-phenyl]-2-methyl-6-phenyl-[1,2,4]triazolo[1,5-a]pyrimidin-7-yl}-(3-methoxy-propyl)-amine,
{5-[4-(1-Amino-cyclobutyl)-phenyl]-2-methyl-6-phenyl-[1,2,4]triazolo[1,5-a]pyrimidin-7-yl}-[2-(4-fluoro-phenyl)-ethyl]-amine,
1-{4-[3-(4-Methansulfonyl-phenyl)-6-phenyl-imidazo[1,2-a]pyrimidin-7-yl]-phenyl}-cyclobutylamine,
{5-[4-(1-Amino-cyclobutyl)-phenyl]-6-phenyl-[1,2,4]triazolo[1,5-a]pyrimidin-2-yl}-(2-methoxy-ethyl)-amine,
{5-[4-(1-amino-cyclobutyl)-phenyl]-6-phenyl-[1,2,4]triazolo[1,5-a]pyrimidin-2-yl}-ethyl-amine,
1-{4-[2-(1-methyl-1H-pyrazol-4-yl)-6-phenyl-[1,2,4]triazolo[1,5-a]pyrimidin-5-yl]-phenyl}-cyclobutylamine,
{5-[4-(1-amino-cyclobutyl)-phenyl]-6-phenyl-[1,2,4]triazolo[1,5-a]pyrimidin-2-yl}-methyl-amine,
{5-[4-(1-Amino-cyclobutyl)-phenyl]-6-phenyl-[1,2,4]triazolo[1,5-a]pyrimidin-2-yl}-phenyl-amine,
{5-[4-(1-amino-cyclobutyl)-phenyl]-6-phenyl-[1,2,4]triazolo[1,5-a]pyrimidin-2-yl}-isopropyl-amine,
{5-[4-(1-amino-cyclobutyl)-phenyl]-6-phenyl-[1,2,4]triazolo[1,5-a]pyrimidin-2-yl}-pyridin-4-ylmethyl-amine,
{5-[4-(1-amino-cyclobutyl)-phenyl]-2-methyl-6-phenyl-[1,2,4]triazolo[1,5-a]pyrimidin-7-yl}-isopropyl-amine,
{5-[4-(1-amino-cyclobutyl)-phenyl]-2-methyl-6-phenyl-[1,2,4]triazolo[1,5-a]pyrimidin-7-yl}-cyclobutyl-amine,
{5-[4-(1-amino-cyclobutyl)-phenyl]-2-methyl-6-phenyl-[1,2,4]triazolo[1,5-a]pyrimidin-7-yl}-cyclopropylmethyl-amine,
{5-[4-(1-amino-cyclobutyl)-phenyl]-2-cyclopropyl-6-phenyl-[1,2,4]triazolo[1,5-a]pyrimidin-7-yl}-methyl-amine,
{5-[4-(1-amino-cyclobutyl)-phenyl]-2-methyl-6-phenyl-[1,2,4]triazolo[1,5-a]pyrimidin-7-yl}-ethyl-amine,
{5-[4-(1-amino-cyclobutyl)-phenyl]-2-methyl-6-phenyl-[1,2,4]triazolo[1,5-a]pyrimidin-7-yl}-cyclopropyl-amine,
{5-[4-(1-amino-cyclobutyl)-phenyl]-2-methyl-6-phenyl-[1,2,4]triazolo[1,5-a]pyrimidin-7-yl}-methyl-amine,
{5-[4-(1-amino-cyclobutyl)-phenyl]-2-methyl-6-phenyl-[1,2,4]triazolo[1,5-a]pyrimidin-7-yl}-(2-methoxy-ethyl)-amine,
4-{5-[4-(1-amino-cyclobutyl)-phenyl]-2-cyclopropyl-6-phenyl-[1,2,4]triazolo[1,5-a]pyrimidin-7-ylamino}cyclohexanol
1-{4-[3-(4-Fluorophenyl)-6-phenyl-imidazo[1,2-a]pyrimidin-7-yl]-phenyl}-cyclobutylamine
(5-{7-[4-(1-Amino-cyclobutyl)-phenyl]-6-phenyl-imidazo[1,2-a]pyrimidin-3-yl}-2-fluoro-phenyl)-methanol
3-{7-[4-(1-Amino-cyclobutyl)-phenyl]-6-phenyl-imidazo[1,2-a]pyrimidin-3-yl}-benzamide
1-{4-[6-Phenyl-3-(1H-pyrazol-4-yl)-imidazo[1,2-a]pyrimidin-7-yl]-phenyl}-cyclobutylamine
   or an N-oxide, a salt, a tautomer or a stereoisomer of said compound, or a salt of said N-oxide, tautomer or stereoisomer.

Another aspect of the invention are the first 35 compounds of the table shown above or an N-oxide, a salt, a tautomer or a stereoisomer of said compound, or a salt of said N-oxide, tautomer or stereoisomer.

One aspect of the present invention are the compounds disclosed in the examples as well as the intermediates as used for their synthesis.

Another aspect of the invention is intermediate (III) wherein all residues are defined as in claims 1-5.

A further aspect of the invention are the stereoisomers and tautomers of the compounds of the present invention, especially those of the compounds disclosed in the examples.

A further aspect of the invention are the N-Oxides of the compounds of the present invention.

Another aspect of the invention are compounds of formula (I), wherein ring C and the pyrimidine to which it is fused form a ring system selected from

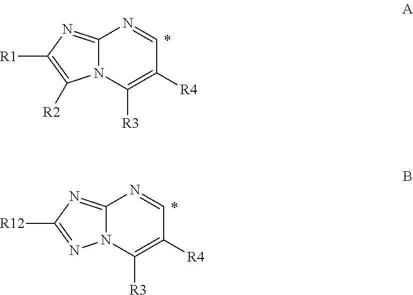

wherein *marks the point of the attachment, resulting in case of ring system A in a compound of formula I-A or in case of a ring system of formula B resulting in a compound of formula I-B.

Another aspect of the invention are compounds of formula (I), wherein ring C and the pyrimidine to which it is fused form a ring system selected from

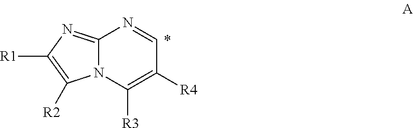

wherein * marks the point of the attachment.

Another aspect of the invention are compounds of formula (I), wherein ring C and the pyrimidine to which it is fused form a ring system selected from

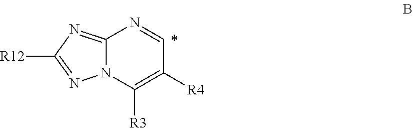

wherein * marks the point of the attachment.

Another aspect of the invention are compounds of formula (I), wherein
R1 is hydrogen, or a group selected from 1-6C-alkyl, 3-7C-cycloalkyl, aryl, heteroaryl,
   wherein said group being optionally substituted, one or more times, identically or differently, with a substituent selected from:
   hydroxy, halogen, 1-6C-alkyl, 1-4C-haloalkyl, 1-6C-alkoxy, —NR8R9, cyano, —C(O)NR8R9, —C(O)OR10, —NHC(O)R11, —NHS(O)₂R11.

Another aspect of the invention are compounds of formula (I), wherein
R1 is hydrogen, or a group selected from 1-3C-alkyl, 3-6C-cycloalkyl, aryl, heteroaryl,
   wherein said group being optionally substituted, one or more times, identically or differently, with a substituent selected from:
   hydroxy, halogen, 1-3C-alkyl, 1-3C-haloalkyl, 1-3C-alkoxy, —NR8R9, cyano, —C(O)NR8R9, —C(O)OR10, —NHC(O)R11, —NHS(O)₂R11.

Another aspect of the invention are compounds of formula (I), wherein
R1 is hydrogen, or 1-6C-alkyl optionally substituted, one or more times, identically or differently, with a substituent selected from:
   hydroxy, halogen, 1-6C-alkoxy,

Another aspect of the invention are compounds of formula (I), wherein

R1 is hydrogen, 1-6C-alkyl, preferably hydrogen or 1-3C-alkyl.

Another aspect of the invention are compounds of formula (I), wherein

R1 is hydrogen, methyl.

Another aspect of the invention are compounds of formula (I), wherein

R1 methyl.

Another aspect of the invention are compounds of formula (I), wherein

R1 is hydrogen.

Another aspect of the invention are compounds of formula (I), wherein
R12 is hydrogen, halogen, —NR13R14, or a group selected from 1-6C-alkyl, 3-7C-cycloalkyl, aryl, heteroaryl,
   wherein said group being optionally substituted, one or more times, identically or differently, with a substituent selected from:
   hydroxy, halogen, 1-6C-alkyl, 1-4C-haloalkyl, 1-6C-alkoxy, —NR8R9, cyano, —C(O)NR8R9, —C(O)OR10, —NHC(O)R11, —NHS(O)₂R11.

Another aspect of the invention are compounds of formula (I), wherein

R12 is hydrogen, 1-6C-alkyl, 3-7C-cycloalkyl, heteroaryl, NR13R14.

Another aspect of the invention are compounds of formula (I), wherein

R12 is hydrogen, 1-3C-alkyl, 3-6C-cycloalkyl, heteroaryl, NR13R14.

Another aspect of the invention are compounds of formula (I), wherein

R12 is hydrogen, methyl, cyclopropyl, N-methyl-pyrazolyl, pyridyl, NR13R14.

Another aspect of the invention are compounds of formula (I), wherein

### R12 is NR13R14.

Another aspect of the invention are compounds of formula (I), wherein

R2 is hydrogen, halogen, cyano, or a group selected from 1-6C-alkyl, 3-7C-cycloalkyl, aryl, heteroaryl, (1-6C-alkyl)-aryl, (1-6C-alkyl)-heteroaryl,
wherein said group being optionally substituted, one or more times, identically or differently, with a substituent selected from:
hydroxy, halogen, 1-6C-alkyl, 1-4C-haloalkyl, 1-6C-alkoxy, —NR8R9, cyano, —C(O)NR8R9, —C(O)OR10, —NHC(O)R11, —NHS(O)₂R11, —S(O)₂R11, —S(O)₂NR8R9.

Another aspect of the invention are compounds of formula (I), wherein

R2 is aryl, heteroaryl,
wherein said group being optionally substituted, one or more times, identically or differently, with a substituent selected from:
hydroxy, halogen, 1-6C-alkyl, 1-4C-haloalkyl, 1-6C-alkoxy, —NR8R9, cyano, —C(O)NR8R9, —C(O)OR10, —NHC(O)R11, —NHS(O)₂R11, —S(O)₂R11, —S(O)₂NR8R9.

Another aspect of the invention are compounds of formula (I), wherein

R2 is hydrogen, aryl, heteroaryl,
wherein said aryl or heteroaryl group being optionally substituted, one or more times, identically or differently, with a substituent selected from:
halogen, 1-6C-alkyl, 1-4C-haloalkyl, 1-6C-hydroxyalkyl, 1-6C-alkoxy, —NR8R9, cyano, —C(O)NR8R9, —C(O)OR10, —NHC(O)R11, —NHS(O)₂R11, —S(O)₂R11, —S(O)₂NR8R9.

Another aspect of the invention are compounds of formula (I), wherein

R2 is hydrogen, aryl, heteroaryl,
wherein said aryl or heteroaryl group being optionally substituted, one or more times, identically or differently, with a substituent selected from:
halogen, 1-3C-alkyl, 1-3C-haloalkyl, 1-3C-hydroxyalkyl, 1-3C-alkoxy, —NR8R9, cyano, —C(O)NR8R9, —C(O)OR10, —NHC(O)R11, —NHS(O)₂R11, —S(O)₂R11, —S(O)₂NR8R9.
R2 is hydrogen, aryl, heteroaryl,
wherein said aryl or heteroaryl group being optionally substituted, one or more times, identically or differently, with a substituent selected from:
halogen, 1-3C-hydroxyalkyl, cyano, —C(O)NR8R9, —S(O)₂R11.

Another aspect of the invention are compounds of formula (I), wherein

R2 is phenyl optionally substituted, one or more times, identically or differently, with a substituent selected from:
hydroxy, halogen, 1-6C-alkyl, 1-4C-haloalkyl, 1-6C-alkoxy, —NR8R9, cyano, —C(O)NR8R9, —C(O)OR10, —NHC(O)R11, —NHS(O)₂R11, —S(O)₂R11, —S(O)₂NR8R9.

Another aspect of the invention are compounds of formula (I), wherein

R2 is hydrogen, halogen, aryl,
wherein said aryl being optionally substituted, one or more times, identically or differently, with a substituent selected from:
halogen, cyano, —S(O)₂R11.

Another aspect of the invention are compounds of formula (I), wherein

R2 is hydrogen, bromine, or, phenyl substituted with cyano, —S(O)₂R11.

Another aspect of the invention are compounds of formula (I), wherein

R3 is hydrogen, 1-6C-alkyl, 3-7C-cycloalkyl, or NR15R16.

Another aspect of the invention are compounds of formula (I), wherein

R3 is hydrogen, 1-6C-alkyl, NR15R16.

Another aspect of the invention are compounds of formula (I), wherein

R3 is hydrogen, 1-3C-alkyl, NR15R16.

Another aspect of the invention are compounds of formula (I), wherein

R3 is hydrogen, methyl, NR15R16.

Another aspect of the invention are compounds of formula (I), wherein

R3 is hydrogen.

Another aspect of the invention are compounds of formula (I), wherein

R3 is methyl

Another aspect of the invention are compounds of formula (I), wherein

### R3 is NR15R16.

Another aspect of the invention are compounds of formula (I), wherein
R4 is phenyl optionally substituted, one or more times, identically or differently, with a substituent selected from: 1-6C-alkyl, halogen, cyano.

Another aspect of the invention are compounds of formula (I), wherein
R4 is phenyl optionally substituted, one or more times, identically or differently, with halogen.

Another aspect of the invention are compounds of formula (I), wherein

R4 is phenyl optionally substituted, one or more times, with fluorine.

Another aspect of the invention are compounds of formula (I), wherein

R4 is unsubstituted phenyl.

Another aspect of the invention are compounds of formula (I), wherein

R5 is hydrogen, halogen.

Another aspect of the invention are compounds of formula (I), wherein

R5 is hydrogen, fluorine.

Another aspect of the invention are compounds of formula (I), wherein

R5 is hydrogen.

Another aspect of the invention are compounds of formula (I), wherein

R6 is hydrogen, 1-6C-alkyl.

Another aspect of the invention are compounds of formula (I), wherein

R6 is hydrogen.

Another aspect of the invention are compounds of formula (I), wherein

R8, R9 which can be the same or different, is hydrogen, 1-4C-alkyl (optionally substituted in the same way of differently one or more times with halogen, hydroxy, mono- or di-1-4C-alkylamino), 1-4C-alkoxy, or 3-7C-cycloalkyl, or,
in the case of —NR8R9, R8 and R9 together with the nitrogen to which they are attached may also form a 3-6C-heterocyclic ring, preferably a 5- or 6-membered heterocyclic ring.

Another aspect of the invention are compounds of formula (I), wherein

R8, R9 which can be the same or different, is hydrogen, 1-4C-alkyl (optionally substituted in the same way of differently one or more times with halogen, hydroxy, mono- or di-(1-4C-alkylamino), 1-4C-alkoxy, or 3-7C-cycloalkyl.

Another aspect of the invention are compounds of formula (I), wherein

R8 is hydrogen and R9 is hydrogen, 1-4C-alkyl (optionally substituted in the same way of differently one or more times with halogen, hydroxy, mono- or di-(1-4C-alkylamino), 1-4C-alkoxy, or 3-7C-cycloalkyl.

Another aspect of the invention are compounds of formula (I), wherein

R8 is hydrogen and R9 is 1-4C-alkyl (optionally substituted in the same way of differently one or more times with halogen, hydroxy, mono- or di-(1-4C-alkylamino), 1-4C-alkoxy, or 3-7C-cycloalkyl.

Another aspect of the invention are compounds of formula (I), wherein

R8 is hydrogen and R9 is 1-4C-alkyl or 3-7C-cycloalkyl.

Another aspect of the invention are compounds of formula (I), wherein

R8 is hydrogen and R9 is 1-4C-alkyl.

Another preferred aspect of the invention are compounds of formula (I), wherein

R8 and R9 are both hydrogen.

Another aspect of the invention are compounds of formula (I), wherein

R10 is hydrogen, 1-6C-alkyl, preferably hydrogen or 1-3C-alkyl.

Another aspect of the invention are compounds of formula (I), wherein

R11 is 1-4C-alkyl (optionally substituted in the same way of differently one or more times with halogen, hydroxy) or 3-7C-cycloalkyl.

Another aspect of the invention are compounds of formula (I), wherein

### R11 is 1-4C-alkyl.

Another aspect of the invention are compounds of formula (I), wherein

R11 is methyl.

Another aspect of the invention are compounds of formula (I), wherein

R13, R14 which can be the same or different, is hydrogen, or a group selected from 1-6C-alkyl, 3-7C-cycloalkyl, aryl, heteroaryl, 1-6C-alkyl-aryl, 1-6C-alkyl-heteroaryl,
wherein said group being optionally substituted, one or more times, identically or differently, with a substituent selected from:
hydroxy, halogen, 1-6C-alkyl, 1-4C-haloalkyl, 1-6C-alkoxy, cyano.

Another aspect of the invention are compounds of formula (I), wherein

R13, R14 which can be the same or different, is hydrogen, or a group selected from 1-6C-alkyl, aryl, 1-6C-alkyl-heteroaryl wherein said group is optionally substituted with 1-6C-alkoxy.

Another aspect of the invention are compounds of formula (I), wherein

R13, R14 which can be the same or different, is hydrogen, or aryl optionally substituted one or more times, identically or differently, with a substituent selected from:
hydroxy, halogen, 1-6C-alkyl, 1-4C-haloalkyl, 1-6C-alkoxy, cyano.

Another aspect of the invention are compounds of formula (I), wherein

R13, R14 which can be the same or different, is hydrogen, or heteroaryl optionally substituted one or more times, identically or differently, with a substituent selected from:
hydroxy, halogen, 1-6C-alkyl, 1-4C-haloalkyl, 1-6C-alkoxy, cyano.

Another aspect of the invention are compounds of formula (I), wherein

R13, R14 which can be the same or different, is hydrogen, methyl, ethyl, —CH(CH3)₂, —(CH₂)₂—OCH₃, phenyl, —CH₂-(pyridyl).

Another aspect of the invention are compounds of formula (I), wherein

R13, R14 which can be the same or different, is hydrogen, methyl, ethyl, —(CH₂)₂—OCH₃, phenyl, —CH₂— (pyridyl).

Another aspect of the invention are compounds of formula (I), wherein

R13 is hydrogen and R14 is hydrogen or a group selected from 1-6C-alkyl, 3-7C-cycloalkyl, aryl, heteroaryl, 1-6C-alkyl-aryl, 1-6C-alkyl-heteroaryl,
wherein said group being optionally substituted, one or more times, identically or differently, with a substituent selected from:
hydroxy, halogen, 1-6C-alkyl, 1-4C-haloalkyl, 1-6C-alkoxy, cyano.

Another aspect of the invention are compounds of formula (I), wherein

R13 is hydrogen and R14 is hydrogen or a group selected from 1-6C-alkyl, aryl, 1-6′-alkyl-heteroaryl wherein said group is optionally substituted with 1-6C-alkoxy.

Another aspect of the invention are compounds of formula (I), wherein

R13 is hydrogen and R14 is aryl optionally substituted one or more times, identically or differently, with a substituent selected from:
hydroxy, halogen, 1-6C-alkyl, 1-4C-haloalkyl, 1-6C-alkoxy, cyano.

Another aspect of the invention are compounds of formula (I), wherein

R13 is hydrogen and R14 is heteroaryl optionally substituted one or more times, identically or differently, with a substituent selected from:
hydroxy, halogen, 1-6C-alkyl, 1-4C-haloalkyl, 1-6C-alkoxy, cyano.

Another aspect of the invention are compounds of formula (I), wherein

R13 is hydrogen and R14 is a group selected from 1-6C-alkyl, aryl, 1-6C-alkyl-heteroaryl wherein said group is optionally substituted with 1-6C-alkoxy

Another aspect of the invention are compounds of formula (I), wherein

R13 is hydrogen and R14 is methyl, ethyl, —(CH₂)₂—OCH₃, phenyl, —CH₂-(pyridyl).

Another aspect of the invention are compounds of formula (I), wherein

R13 is hydrogen and R14 is methyl, ethyl, —CH(CH3)2, —(CH₂)₂—OCH₃, phenyl, —CH₂— (pyridyl).

Another aspect of the invention are compounds of formula (I), wherein

R15, R16 which can be the same or different, is hydrogen, or a group selected from 1-6C-alkyl, 3-7C-cycloalkyl, 1-6C-alkyl-aryl, 1-6C-alkyl-heteroaryl,
wherein said group being optionally substituted, one or more times, identically or differently, with a substituent selected from:
hydroxy, halogen, 1-6C-alkyl, 1-4C-haloalkyl, 1-6C-alkoxy, cyano, 3-7-cycloalkyl, heterocyclyl, —C(O)NR10R11, —C(O)OR10, —NHC(O)R11, —NHS(O)₂R11, or, in the case of —NR15R16, R15 and R16 together with the nitrogen to which they are attached may also form a 3-6C-heterocyclic ring.

Another aspect of the invention are compounds of formula (I), wherein

R15, R16 which can be the same or different, is hydrogen, or a group selected from 1-3C-alkyl, 3-6C-cycloalkyl, 1-3C-alkyl-aryl, 1-3C-alkyl-heteroaryl,
wherein said group being optionally substituted, one or more times, identically or differently, with a substituent selected from:
hydroxy, halogen, 1-3C-alkyl, 1-3C-haloalkyl, 1-3C-alkoxy, cyano, 3-6-cycloalkyl, heterocyclyl, —C(O)NR10R11, —C(O)OR10, —NHC(O)R11, —NHS(O)₂R11, or,
in the case of —NR15R16, R15 and R16 together with the nitrogen to which they are attached may also form a 3-6C-heterocyclic ring.

Another aspect of the invention are compounds of formula (I), wherein

R15, R16 which can be the same or different, is hydrogen, or a group selected from 1-6C-alkyl, 3-7C-cycloalkyl, (1-6C-alkyl)-aryl, (1-6C-alkyl)heteroaryl,
wherein said group being optionally substituted, one or more times, identically or differently, with a substituent selected from:
hydroxy, halogen, 1-6C-alkyl, 1-4C-haloalkyl, 1-6C-alkoxy, cyano, 3-7-cycloalkyl, heterocyclyl, —C(O)OR10.

Another aspect of the invention are compounds of formula (I), wherein

R15, R16 which can be the same or different, is hydrogen, or a group selected from 1-6C-alkyl, 3-7C-cycloalkyl, (1-6C-alkyl)-aryl, (1-6C-alkyl)-heteroaryl,
wherein said group being optionally substituted, one or more times, identically or differently, with a substituent selected from:
halogen, 1-6C-alkyl, 1-6C-alkoxy, 3-7C-cycloalkyl, —C(O)OR10.

Another aspect of the invention are compounds of formula (I), wherein

R15, R16 which can be the same or different, is hydrogen, methyl, isopropyl, cyclopropyl, cyclobutyl optionally substituted by —C(O)OCH₂CH₃, or 1-4C-alkyl substituted with methoxy, cyclopropyl, 4-fluoro-phenyl, N-methyl-pyrazole.

Another aspect of the invention are compounds of formula (I), wherein

R15 is hydrogen and R16 is hydrogen, or a group selected from 1-3C-alkyl, 3-6C-cycloalkyl, 1-3C-alkyl-aryl, 1-3C-alkyl-heteroaryl,
wherein said group being optionally substituted, one or more times, identically or differently, with a substituent selected from:
hydroxy, halogen, 1-3C-alkyl, 1-3C-haloalkyl, 1-3C-alkoxy, cyano, 3-6-cycloalkyl, heterocyclyl, —C(O)NR10R11, —C(O)OR10, —NHC(O)R11, —NHS(O)₂R11, or, in the case of —NR15R16, R15 and R16 together with the nitrogen to which they are attached may also form a 3-6C-heterocyclic ring.

Another aspect of the invention are compounds of formula (I), wherein

R15 is hydrogen and R16 is a group selected from 1-6C-alkyl, 3-7C-cycloalkyl, (1-6C-alkyl)-aryl, (1-6C-alkyl)-heteroaryl,
wherein said group being optionally substituted, one or more times, identically or differently, with a substituent selected from:
halogen, 1-6C-alkyl, 1-6C-alkoxy, 3-7C-cycloalkyl, —C(O)OR10.

Another aspect of the invention are compounds of formula (I), wherein

R15 is hydrogen and R16 is a group selected from 1-6C-alkyl, 3-6C-cycloalkyl, (1-6C-alkyl)-aryl,
wherein said group being optionally substituted, one or more times, identically or differently, with a substituent selected from: hydroxy, halogen, 1-3C-alkoxy, 3-5C-cycloalkyl, —C(O)OR10.

Another aspect of the invention are compounds of formula (I), wherein

R15 is hydrogen and R16 is a group selected from 1-3C-alkyl, 3-6C-cycloalkyl, (1-3C-alkyl)-aryl,
wherein said group being optionally substituted, one or more times, identically or differently, with a substituent selected from: hydroxy, halogen, 1-3C-alkoxy, 3-5C-cycloalkyl, —C(O)O(1-3C-alkyl).

Another aspect of the invention are compounds of formula (I), wherein

R15 is hydrogen and R16 is methyl, isopropyl, cyclopropyl, cyclobutyl optionally substituted by —C(O)OCH₂CH₃, or 1-4C-alkyl substituted with methoxy, cyclopropyl, 4-fluoro-phenyl, N-methyl-pyrazole.
R15 is hydrogen and

### R16 is

methyl, isopropyl, cyclopropyl, cyclobutyl, cyclohexyl optionally substituted with hydroxy, —C(O)OCH₂CH₃; or
1-4C-alkyl optionally substituted with methoxy, cyclopropyl, 4-fluoro-phenyl.

Another aspect of the invention are compounds of formula (I), wherein

X is —(CH₂)ₙ—.

Another aspect of the invention are compounds of formula (I), wherein

n is 0, 1, 2, or 3.

Another aspect of the invention are compounds of formula (I), wherein

n is 0, 1, or 2.

Another aspect of the invention are compounds of formula (I), wherein

n is 0, or 1.

Another aspect of the invention are compounds of formula (I), wherein

n is 0.

A preferred aspect of the invention are compounds of formula (I), wherein

n is 1 or 2.

Another aspect of the invention are compounds of formula (I), wherein

n is 2.

Another aspect of the invention are compounds of formula (I), wherein

### Y is —CH₂—, —CH(OH)—.

A preferred aspect of the invention are compounds of formula (I), wherein

### Y is —CH₂—.

In another embodiment of the above-mentioned aspects, the invention relates to compounds of formula (I), wherein R6 is hydrogen, R5 is hydrogen and R4 is an unsubstituted phenyl moiety.

In a further embodiment of the above-mentioned aspects, the invention relates to compounds of formula (I), wherein R5 is hydrogen and R4 is an unsubstituted phenyl moiety.

In a further embodiment of the above-mentioned aspects, the invention relates to compounds of formula (I), wherein R5 is hydrogen, R4 is an unsubstituted phenyl moiety and R3 is hydrogen.

In a further embodiment of the above-mentioned aspects, the invention relates to compounds of formula (I), wherein R5 is hydrogen, R4 is an unsubstituted phenyl moiety and R3 is hydrogen.

### DEFINITIONS

Unless defined otherwise in the claims the constituents defined below can optionally be substituted, one or more times, identically or differently, with a substituent selected from:

hydroxy, halogen, cyano, 1-6C-alkyl, 1-4C-haloalkyl, 1-6C-alkoxy, —NR8R9, cyano, (═O), —C(O)NR8R9, —C(O)OR10, —NHC(O)R11, —NHS(O)₂R11. An alkyl constituent being substituted more times by halogen includes also a completely halogenated alkyl moiety such as e.g. CF3.

Should a constituent be composed of more than one part, e.g. —O-(1-6Calkyl)-3-7C-cycloalkyl, the position of a possible substituent can be at any of these parts at any suitable position. A hyphen at the beginning of the constituent marks the point of attachment to the rest of the molecule. Should a ring be substituted the substitutent could be at any suitable position of the ring, also on a ring nitrogen atom.

The term “comprising” when used in the specification includes “consisting of”.

If it is referred to “as mentioned above” or “mentioned above” within the description it is referred to any of the disclosures made within the specification in any of the preceding pages.

“suitable” within the sense of the invention means chemically possible to be made by methods within the knowledge of a skilled person.

“1-6C-alkyl” is a straight-chain or branched alkyl group having 1 to 6 carbon atoms. Examples are methyl, ethyl, n propyl, iso-propyl, n butyl, iso-butyl, sec-butyl and tert-butyl, pentyl, hexyl, preferably 1-4 carbon atoms (1-4C-alkyl), more preferably 1-3 carbon atoms (1-3C-alkyl). Other alkyl constituents mentioned herein having another number of carbon atoms shall be defined as mentioned above taking into account the different length of their chain. Those parts of constituents containing an alkyl chain as a bridging moiety between two other parts of the constituent which usually is called an “alkylene” moiety is defined in line with the definition for alkyl above including the preferred length of the chain e.g. methylen, ethylene, n-propylen, iso-propylen, n-butylen, isobutylene, tert-butylen.

For the term “1-6C-hydroxyalkyl” it is understood that it includes the chain definition of 1-6C-alkyl and the hydroxyalkyl constituent contains one hydroxy group at any possible position of the chain.

“Mono- or di-1-4C-alkylamino” radicals contain in addition to the nitrogen atom, independently one or two of the above mentioned 1-4C-alkyl radicals. Examples are the methyamino, the ethylamino, the isopropylamino, the dimethylamino, the diethylamino and the diisopropylamino radical.

“Halogen” within the meaning of the present invention is iodine, bromine, chlorine or fluorine, preferably “halogen” within the meaning of the present invention is chlorine or fluorine.

“1-4C-Haloalkyl” is a straight-chain or branched alkyl group having 1 to 4 carbon atoms in which at least one hydrogen is substituted by a halogen atom. Examples are chloromethyl or 2-bromoethyl. For a partially or completely fluorinated C1-C4-alkyl group, the following partially or completely fluorinated groups are consid-ered, for example: fluoromethyl, difluoromethyl, trifluoromethyl, fluoroethyl, 1,1-difluoroethyl, 1,2-difluoroethyl, 1,1,1-trifluoroethyl, tetrafluoroethyl, and penta-fluoroethyl, whereby fluoromethyl, difluoromethyl, trifluoromethyl, fluoroethyl, 1,1-difluoroethyl, or 1,1,1-trifluoroethyl are preferred. Partially or completely fluorinated C1-C4-alkyl groups are considered to be encompassed by the term 1-4C-haloalkyl.

“1-6C-Alkoxy” represents radicals, which in addition to the oxygen atom, contain a straight-chain or bran [symbol] ched alkyl radical having 1 to 6 carbon atoms. Examples which may be mentioned are the hexoxy, pentoxy, butoxy, iso [symbol] butoxy, sec-butoxy, tert-butoxy, pro-poxy, isopropoxy, ethoxy and methoxy radicals, preferred are methoxy, ethoxy, propoxy, isopropoxy.

“3-7C-Cycloalkyl” stands for cyclopropyl, cyclobutyl, cyclopentyl, cyclohexyl or cycloheptyl, preferably cyclopropyl.

“3-7C-Heterocyclyl”, or “heterocyclyl” represents a mono- or polycyclic, preferably mono- or bicyclic, more preferably monocyclic, nonaromatic heterocyclic radical containing, 4 to 10, preferably 4 to 7, more preferably 5-6 ring atoms, and up to 3, preferably up to 2, hetero atoms and/or hetero groups from the series consisting of N, O, S, SO, SO₂, preferably a nitrogen atom or an oxygen atom, in a special embodiment oxygen. The heterocyclyl radicals can be saturated or partially unsaturated and, unless stated otherwise, may be optionally substituted, one or more times, identically or differently, with a substituent selected from: 1-4C-alkyl, 1-4C-haloalkyl, 1-4C-alkoxy, hydroxy, fluorine, whereby the 1-4C-alkyl may be optionally further substituted with hydroxy. Particularly preferred heterocyclic radicals are 4- to 7-membered monocyclic saturated heterocyclyl radicals having up to two hetero atoms from the series consisting of O, N and S. The following may be mentioned by way of example and by preference: oxetanyl, tetrahydrofuranyl, azetidinyl, 3-hydroxyazetidinyl, 3-fluoroazetidinyl, 3,3-difluoroazetidinyl, pyrrolidinyl, 3-hydroxypyrrolidinyl, pyrrolinyl, piperidinyl, 3-hydroxypiperidinyl, 4-hydroxypiperidinyl, 3-fluoropiperidinyl, 3,3-difluoropiperidinyl, 4-fluoropiperidinyl, 4,4-difluoropiperidinyl, piperazinyl, N-methyl-piperazinyl, N-(2-hydroxyethyl)-piperazinyl, morpholinyl, thiomorpholinyl, azepanyl, homopiperazinyl, N-methyl-homopiperazinyl.

In the case of —NR8R9 or NR15R16, when R8 and R9 together with the nitrogen atom to which they are attached or R15 and R16 together with the nitrogen atom to which they are attached form a 3-6C-heterocyclic ring, the term “3-6C-heterocyclic ring” includes all saturated heterocyclic rings containing 4 to 7 ring atoms and having 1 or 2 nitrogen atoms, or 1 nitrogen atom and 1 oxygen atom. The 3-6C-heterocyclic ring may be optionally substituted one or more times, identically or differently, with a substituent selected from: 1-4C-alkyl, 1-4C-haloalkyl, 1-4C-alkoxy, hydroxy, fluorine, whereby the 1-4C-alkyl may be optionally further substituted with hydroxy. Preferred examples are azetidine, 3-hydroxyazetidine, 3-fluoroazetidine, 3,3-difluoroazetidine, pyrrolidine, 3-hydroxypyrrolidine, piperidine, 3-hydroxypiperidine, 4-hydroxypiperidine, 3-fluoropiperidine, 3,3-difluoropiperidine, 4-fluoropiperidine, 4,4-difluoropiperidine, piperazine, N-methyl-piperazine, N-(2-hydroxyethyl)-piperazine, morpholine.

“Aryl” represents a mono-, or bicyclic aromatic carbocyclic radical having, as a rule, 6 to 10 carbon atoms; by way of example phenyl or naphthyl. Phenyl is preferred. The aryl moiety can be substituted one or more times, identically or differently by hydroxy, halogen, cyano, 1-6C-alkyl, 1-4C-haloalkyl, 1-6C-alkoxy, —NR8R9, cyano, —C(O)NR8R9, —C(O)OR10, —NHC(O)R11, —NHS(O)₂R11.

The term “1-6C-alkyl-aryl” or “-(1-6C-alkylen)-aryl” represents an aryl radical as defined above which is connected to the rest of the molecule via a straight or branched alkyl chain, preferably —(CH₂)-aryl. Benzyl, is particularly preferred.

The term “heteroaryl” represents a monocyclic 5- or 6-membered aromatic heterocycle comprised without being restricted thereto, the 5-membered heteroaryl radicals furyl, thienyl, pyrrolyl, oxa-zolyl, isoxazolyl, thiazolyl, isothiazolyl, imidazolyl, pyrazolyl, triazolyl (1,2,4-triazolyl, 1,3,4-triazolyl or 1,2,3-triazolyl), thiadiazolyl (1,3,4-thiadiazolyl, 1,2,5-thiadiazolyl, 1,2,3-thiadiazolyl or 1,2,4-thiadiazolyl) and oxadiazolyl (1,3,4-oxadiazolyl, 1,2,5-oxadiazolyl, 1,2,3-oxadiazolyl or 1,2,4-oxadiazolyl), as well as the 6-membered heteroaryl radicals pyridinyl, pyrimidinyl, pyrazinyl and pyridazinyl. Preferred 5- or 6-membered heteroaryl radicals are furanyl, thienyl, pyrrolyl, thiazolyl, oxazolyl, thiadiazolyl, oxadiazolyl, pyridinyl, pyrimidinyl, pyrazinyl or pyridazinyl. More preferred 5- or 6-membered heteroaryl radicals are furan-2-yl, thien-2-yl, pyrrol-2-yl, thiazolyl, oxazolyl, 1,3,4-thiadiazolyl, 1,3,4-oxadiazolyl, pyridin-2-yl, pyridin-4-yl, pyrimidin-2-yl, pyrimidin-4-yl, pyrazin-2-yl or pyridazin-3-yl.

The term “(1-6C-alkyl)-heteroaryl” or “(1-6C-alkylen)-heteroaryl” represents a heteroaryl radical as defined above which is connected to the rest of the molecule via a straight or branched alkyl chain, preferably 1-4C-alkyl-heteroaryl, whereby —(CH₂)-heteroaryl is particularly preferred.

The NR8R9 group includes, for example, NH2, N(H)CH3, N(CH3)2, N(H)CH2CH3 and N(CH3)CH2CH3. In the case of —NR8R9, when R8 and R9 together with the nitrogen atom to which they are attached form a 3-6C-heterocyclic ring, the term “3-6C-heterocyclic ring” is defined above.

The NH(CO)R11 group includes for example NH(CO)CH3, NH(CO)C2H5, NH(CO)C3H7, NH(CO)CH(CH3)₂.

The NHS(O)₂R11 group includes for example NHS(O)2CH3, NHS(O)2C2H5, NHS(O)2C3H7, NHS(O)2CH(CH3)2.

The C(O)NR8R9 group includes, for example, C(O)NH2, C(O)N(H)CH3, C(O)N(CH3)2, C(O)N(H)CH2CH3, C(O)N(CH3)CH2CH3 or C(O)N(CH2CH3)2. In the case of —NR8R9, when R8 and R9 together with the nitrogen atom to which they are attached form a 3-6C-heterocyclic ring, the term “3-6C-heterocyclic ring” is defined above.

The C(O)OR10 group includes for example C(O)OH, C(O)OCH3, C(O)OC2H5, C(O)C3H7, C(O)CH(CH3)₂, C(O)OC4H9, C(O)OC5H11, C(O)OC6H13; for C(O)O(1-6C-alkyl) the alkyl part may be straight or branched.

In general and unless otherwise mentioned, the heteroarylic or heteroarylenic radicals include all the possible isomeric forms thereof, e.g. the positional isomers thereof. Thus, for some illustrative non-restricting example, the term pyridinyl or pyridinylene includes pyridin-2-yl, pyridin-2-ylene, pyridin-3-yl, pyridin-3-ylene, pyridin-4-yl and pyridin-4-ylene; or the term thienyl or thienylene includes thien-2-yl, thien-2-ylene, thien-3-yl and thien-3-ylene.

Constituents which are optionally substituted as stated herein, may be substi-tuted, unless otherwise noted, one or more times, independently from one another at any possible position. When any variable occurs more than one time in any constituent, each definition is independent.

In case of R2, R13, R14, R15, it is understood that the groups selected from (1-6C-alkylen)-aryl, or (1-6C-alkylen)-heteroaryl, are preferably either not substituted within the (1-6C-alkylen) part, or the (1-6C-alkylen) part is optionally substituted with one or two fluorine atoms.

The heteroarylic, heteroarylenic, or heterocyclic groups mentioned herein may be substituted by their given substituents or parent molecular groups, unless otherwise noted, at any possible position, such as e.g. at any substitutable ring carbon or ring nitrogen atom. Analogously it is being understood that it is possible for any heteroaryl or heterocyclyl group to be attached to the rest of the molecule via any suitable atom if chemically suitable. Unless otherwise noted, any heteroatom of a heteroarylic or heteroarylenic ring with unsatisfied valences mentioned herein is assumed to have the hydrogen atom(s) to satisfy the valences. Unless otherwise noted, rings containing quaternizable amino- or imino-type ring nitrogen atoms (—N═) may be preferably not quaternized on these amino- or imino-type ring nitrogen atoms by the mentioned substituents or parent molecular groups.

In the context of the properties of the compounds of the present invention the term “pharmacokinetic profile” means one single parameter or a combination thereof including permeability, bioavailability, exposure, and pharmacodynamic parameters such as duration, or magnitude of pharmacological effect, as measured in a suitable experiment.

Salts of the compounds according to the invention include all inorganic and organic acid addition salts and salts with bases, especially all pharmaceutically acceptable inorganic and organic acid addition salts and salts with bases, particularly all pharmaceutically acceptable inorganic and organic acid addition salts and salts with bases customarily used in pharmacy.

One aspect of the invention are salts of the compounds according to the invention including all inorganic and organic acid addition salts, especially all pharmaceutically acceptable inorganic and organic acid addition salts, particularly all pharmaceutically acceptable inorganic and organic acid addition salts customarily used in pharmacy. Another aspect of the invention are the salts with di- and tricarboxylic acids.

Examples of acid addition salts include, but are not limited to, hydrochlorides, hydrobromides, phosphates, nitrates, sulfates, salts of sulfamic acid, formates, acetates, propionates, citrates, D-gluconates, benzoates, 2-(4-hydroxybenzoyl)-benzoates, butyrates, salicylates, sulfosalicylates, lactates, maleates, laurates, malates, fumarates, succinates, oxalates, malonates, pyruvates, acetoacetates, tartarates, stearates, benzensulfonates, toluenesulfonates, methanesulfonates, trifluoromethansulfonates, 3-hydroxy-2-naphthoates, benzenesulfonates, naphthalinedisulfonates and trifluoroacetates.

Examples of salts with bases include, but are not limited to, lithium, sodium, potassium, calcium, aluminum, magnesium, titanium, meglumine, ammonium, salts optionally derived from NH₃ or organic amines having from 1 to 16 C-atoms such as e.g. ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylendiamine, N-methylpiperindine and guanidinium salts.

The salts include water-insoluble and, particularly, water-soluble salts.

According to the person skilled in the art the compounds of formula (I) according to this invention as well as their salts may contain, e.g. when isolated in crystalline form, varying amounts of solvents. Included within the scope of the invention are therefore all solvates and in particular all hydrates of the compounds of formula (I) according to this invention as well as all solvates and in particular all hydrates of the salts of the compounds of formula (I) according to this invention.

One aspect of the invention are the salts as disclosed in the experimental section.

Optionally, compounds of the formula (I) can be converted into their N-oxides. The N-oxide may also be introduced by way of an intermediate. N-oxides may be prepared by treating an appropriate precursor with an oxidizing agent, such as meta-chloroperbenzoic acid, in an appropriate solvent, such as dichloromethane, at suitable temperatures, such as from 0° C. to 40° C., whereby room temperature is generally preferred. Further corresponding processes for forming N-oxides are customary for the skilled person.

The invention also includes all suitable isotopic variations of a compound of the invention. An isotopic variation of a compound of the invention is defined as one in which at least one atom is replaced by an atom having the same atomic number but an atomic mass different from the atomic mass usually or predominantly found in nature. Examples of isotopes that can be incorporated into a compound of the invention include isotopes of hydrogen, carbon, nitrogen, oxygen, phosphorus, sulphur, fluorine, chlorine, bromine and iodine, such as ²H (deuterium), ³H (tritium), ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁹ I and ¹³¹I, respectively. Certain isotopic variations of a compound of the invention, for example, those in which one or more radioactive isotopes such as ³H or ¹⁴C are incorporated, are useful in drug and/or substrate tissue distribution studies. Tritiated and carbon-14, i.e., ¹⁴C, isotopes are particularly preferred for their ease of preparation and detectability. Further, substitution with isotopes such as deuterium may afford certain therapeutic advantages resulting from greater metabolic stability, for example, increased in vivo half-life or reduced dosage requirements and hence may be preferred in some circumstances. Isotopic variations of a compound of the invention can generally be prepared by conventional procedures known by a person skilled in the art such as by the illustrative methods or by the preparations described in the examples hereafter using appropriate isotopic variations of suitable reagents.

The term “combination” in the present invention is used as known to persons skilled in the art and may be present as a fixed combination, a non-fixed combination or kit-of-parts.

A “fixed combination” in the present invention is used as known to persons skilled in the art and is defined as a combination wherein the said first active ingredient and the said second active ingredient are present together in one unit dosage or in a single entity. One example of a “fixed combination” is a pharmaceutical composition wherein the said first active ingredient and the said second active ingredient are present in admixture for simultaneous administration, such as in a formulation. Another example of a “fixed combination” is a pharmaceutical combination wherein the said first active ingredient and the said second active ingredient are present in one unit without being in admixture.

A non-fixed combination or “kit-of-parts” in the present invention is used as known to persons skilled in the art and is defined as a combination wherein the said first active ingredient and the said second active ingredient are present in more than one unit. One example of a non-fixed combination or kit-of-parts is a combination wherein the said first active ingredient and the said second active ingredient are present separately. The components of the non-fixed combination or kit-of-parts may be administered separately, sequentially, simultaneously, concurrently or chronologically staggered.

The term “(chemotherapeutic) anti-cancer agents”, includes but is not limited to (i) alkylating/carbamylating agents such as Cyclophosphamid (Endoxan®), Ifosfamid (Holoxan®), Thiotepa (Thiotepa Lederle®), Melphalan (Alkeran®), or chloroethylnitrosourea (BCNU); (ii) platinum derivatives like cis-platin (Platinex® BMS), oxaliplatin (Eloxatin®), satraplatin or carboplatin (Cabroplat® BMS); (iii) antimitotic agents/tubulin inhibitors such as vinca alkaloids (vincristine, vinblastine, vinorelbine), taxanes such as Paclitaxel (Taxol®), Docetaxel (Taxotere®) and analogs as well as new formulations and conjugates thereof (like the nanoparticle formulation Abraxane® with paclitaxel bound to albumin), epothilones such as Epothilone B (Patupilone®), Azaepothilone (Ixabepilone®) or Sagopilone; (iv) topoisomerase inhibitors such as anthracyclines (exemplified by Doxorubicin/Adriblastin®), epipodophyllotoxines (examplified by Etoposide/Etopophos®) and camptothecin and camptothecin analogs (exemplified by Irinotecan/Camptosar® or Topotecan/Hycamtin®); (v) pyrimidine antagonists such as 5-fluorouracil (5-FU), Capecitabine (Xeloda®), Arabinosylcytosine/Cytarabin (Alexan®) or Gemcitabine (Gemzar®); (vi) purin antagonists such as 6-mercaptopurine (Puri-Nethol®), 6-thioguanine or fludarabine (Fludara®) and (vii) folic acid antagonists such as methotrexate (Farmitrexat®) or premetrexed (Alimta®).

The term “target specific anti-cancer agent”, includes but is not limited to (i) kinase inhibitors such as e.g. Imatinib (Glivec®), ZD-1839/Gefitinib (Iressa®), Bay43-9006 (Sorafenib, Nexavar®), SU11248/Sunitinib (Sutent®), OSI-774/Erlotinib (Tarceva®), Dasatinib (Sprycel®), Lapatinib (Tykerb®), or, see also below, Vatalanib, Vandetanib (Zactima®) or Pazopanib; (ii) proteasome inhibitors such as PS-341/Bortezumib (Velcade®); (iii) histone deacetylase inhibitors like SAHA (Zolinza®), PXD101, MS275, MGCD0103, Depsipeptide/FK228, NVP-LBH589, Valproic acid (VPA), CRA/PCI-24781, ITF2357, SB939 and butyrates (iv) heat shock protein 90 inhibitors like 17-allylaminogeldanamycin (17-AAG) or 17-dimethylaminogeldanamycin (17-DMAG); (v) vascular targeting agents (VTAs) like combretastin A4 phosphate or AVE8062/AC7700 and anti-angiogenic drugs like the VEGF antibodies, such as Bevacizumab (Avastin®), or KDR tyrosine kinase inhibitors such as PTK787/ZK222584 (Vatalanib®) or Vandetanib (Zactima®) or Pazopanib; (vi) monoclonal antibodies such as Trastuzumab (Herceptin®), Rituximab (MabThera/Rituxan®), Alemtuzumab (Campath®), Tositumomab (Bexxar®), C225/Cetuximab (Erbitux®), Avastin (see above) or Panitumumab (Vectibix®) as well as mutants and conjugates of monoclonal antibodies, e.g. Gemtuzumab ozogamicin (Mylotarg®) or Ibritumomab tiuxetan (Zevalin®), and antibody fragments; (vii) oligonucleotide based therapeutics like G-3139/Oblimersen (Genasense®) or the DNMT1 inhibitor MG98; (viii) Toll-like receptor/TLR 9 agonists like Promune®, TLR 7 agonists like Imiquimod (Aldara®) or Isatoribine and analogues thereof, or TLR 7/8 agonists like Resiquimod as well as immunostimulatory RNA as TLR 7/8 agonists; (ix) protease inhibitors; (x) hormonal therapeutics such as anti-estrogens (e.g. Tamoxifen or Raloxifen), anti-androgens (e.g. Flutamide or Casodex), LHRH analogs (e.g. Leuprolide, Goserelin or Triptorelin) and aromatase inhibitors (e.g. Femara, Arimedex or Aromasin).

Other “target specific anti-cancer agents” include bleomycin, retinoids such as all-trans retinoic acid (ATRA), DNA methyltransferase inhibitors such as 5-Aza-2′-deoxycytidine (Decitabine, Dacogen®) and 5-azacytidine (Vidaza®), alanosine, cytokines such as interleukin-2, interferons such as interferon α2 or interferon-γ, bcl2 antagonists (e.g. ABT-737 or analogs), death receptor agonists, such as TRAIL, DR4/5 agonistic antibodies, FasL and TNF-R agonists (e.g. TRAIL receptor agonists like mapatumumab or lexatumumab).

Specific examples of anti-cancer agents include, but are not limited to 5 FU, actinomycin D, ABARELIX, ABCIXIMAB, ACLARUBICIN, ADAPALENE, ALEMTUZUMAB, ALTRETAMINE, AMINOGLUTETHIMIDE, AMIPRILOSE, AMRUBICIN, ANASTROZOLE, ANCITABINE, ARTEMISININ, AZATHIOPRINE, BASILIXIMAB, BENDAMUSTINE, BEVACIZUMAB, BEXXAR, BICALUTAMIDE, BLEOMYCIN, BORTEZOMIB, BROXURIDINE, BUSULFAN, CAMPATH, CAPECITABINE, CARBOPLATIN, CARBOQUONE, CARMUSTINE, CETRORELIX, CHLORAMBUCIL, CHLORMETHINE, CISPLATIN, CLADRIBINE, CLOMIFENE, CYCLOPHOSPHAMIDE, DACARBAZINE, DACLIZUMAB, DACTINOMYCIN, DASATINIB, DAUNORUBICIN, DECITABINE, DESLORELIN, DEXRAZOXANE, DOCETAXEL, DOXIFLURIDINE, DOXORUBICIN, DROLOXIFENE, DROSTANOLONE, EDELFOSINE, EFLORNITHINE, EMITEFUR, EPIRUBICIN, EPITIOSTANOL, EPTAPLATIN, ERBITUX, ERLOTINIB, ESTRAMUSTINE, ETOPOSIDE, EXEMESTANE, FADROZOLE, FINASTERIDE, FLOXURIDINE, FLUCYTOSINE, FLUDARABINE, FLUOROURACIL, FLUTAMIDE, FORMESTANE, FOSCARNET, FOSFESTROL, FOTEMUSTINE, FULVESTRANT, GEFITINIB, GENASENSE, GEMCITABINE, GLIVEC, GOSERELIN, GUSPERIMUS, HERCEPTIN, IDARUBICIN, IDOXURIDINE, IFOSFAMIDE, IMATINIB, IMPROSULFAN, INFLIXIMAB, IRINOTECAN, IXABEPILONE, LANREOTIDE, LAPATINIB, LETROZOLE, LEUPRORELIN, LOBAPLATIN, LOMUSTINE, LUPROLIDE, MELPHALAN, MERCAPTOPURINE, METHOTREXATE, METUREDEPA, MIBOPLATIN, MIFEPRISTONE, MILTEFOSINE, MIRIMOSTIM, MITOGUAZONE, MITOLACTOL, MITOMYCIN, MITOXANTRONE, MIZORIBINE, MOTEXAFIN, MYLOTARG, NARTOGRASTIM, NEBAZUMAB, NEDAPLATIN, NILUTAMIDE, NIMUSTINE, OCTREOTIDE, ORMELOXIFENE, OXALIPLATIN, PACLITAXEL, PALIVIZUMAB, PANITUMUMAB, PATUPILONE, PAZOPANIB, PEGASPARGASE, PEGFILGRASTIM, PEMETREXED, PENTETREOTIDE, PENTOSTATIN, PERFOSFAMIDE, PIPOSULFAN, PIRARUBICIN, PLICAMYCIN, PREDNIMUSTINE, PROCARBAZINE, PROPAGERMANIUM, PROSPIDIUM CHLORIDE, RALOXIFEN, RALTITREXED, RANIMUSTINE, RANPIRNASE, RASBURICASE, RAZOXANE, RITUXIMAB, RIFAMPICIN, RITROSULFAN, ROMURTIDE, RUBOXISTAURIN, SAGOPILONE, SARGRAMOSTIM, SATRAPLATIN, SIROLIMUS, SOBUZOXANE, SORAFENIB, SPIROMUSTINE, STREPTOZOCIN, SUNITINIB, TAMOXIFEN, TASONERMIN, TEGAFUR, TEMOPORFIN, TEMOZOLOMIDE, TENIPOSIDE, TESTOLACTONE, THIOTEPA, THYMALFASIN, TIAMIPRINE, TOPOTECAN, TOREMIFENE, TRAIL, TRASTUZUMAB, TREOSULFAN, TRIAZIQUONE, TRIMETREXATE, TRIPTORELIN, TROFOSFAMIDE, UREDEPA, VALRUBICIN, VATALANIB, VANDETANIB, VERTEPORFIN, VINBLASTINE, VINCRISTINE, VINDESINE, VINORELBINE, VOROZOLE, ZEVALIN and ZOLINZA.

The compounds according to the invention and their salts can exist in the form of tautomers which are included in the embodiments of the invention.

The compounds of the invention may, depending on their structure, exist in different stereoisomeric forms. These forms include configurational isomers or optionally conformational isomers (enantiomers and/or diastereoisomers including those of atropisomers). The present invention therefore includes enantiomers, diastereoisomers as well as mixtures thereof. From those mixtures of enantiomers and/or disastereoisomers pure stereoisomeric forms can be isolated with methods known in the art, preferably methods of chromatography, especially high pressure liquid chromatography (HPLC) using achiral or chiral phase. The invention further includes all mixtures of the stereoisomers mentioned above independent of the ratio, including the racemates.

Some of the compounds and salts according to the invention may exist in different crystalline forms (polymorphs) which are within the scope of the invention.

Furthermore, derivatives of the compounds of formula (I) and the salts thereof which are converted into a compound of formula (I) or a salt thereof in a biological system (bioprecursors or pro-drugs) are covered by the invention. Said biological system is e.g. a mammalian organism, particularly a human subject. The bioprecursor is, for example, converted into the compound of formula (I) or a salt thereof by metabolic processes.

The intermediates used for the synthesis of the compounds of claims 1-6 as described below, as well as their use for the synthesis of the compounds of claims 1-6, are one further aspect of the present invention. Preferred intermediates are the Intermediate Examples as disclosed below.

The Compounds According to the Invention can be Prepared as Follows.

The compounds according to the invention can be prepared according to the following scheme,

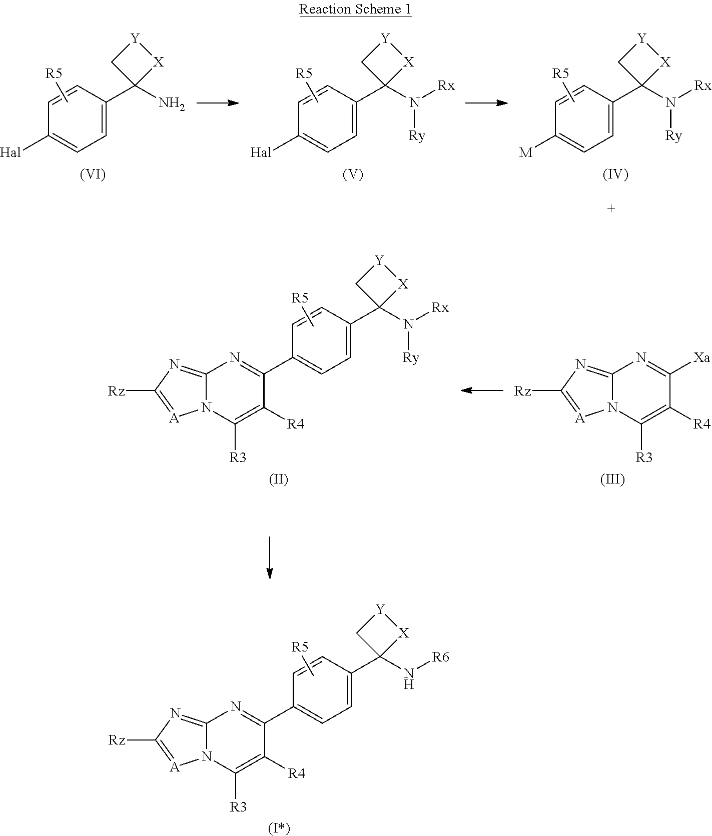

in which it is understood that the general formula (1*) in reaction scheme 1 represents the compounds of general formula (I) according to claim 1.

The compounds according to the invention can be prepared according to reaction scheme 1 wherein X, Y, R1, R2, R3, R4, R5, R6 and R12 have the meanings defined in claim 1, whereby A is N or CR2; Rz has the meaning of R1 or R12; Rx has the meaning of R6 and may also be a protecting group; Ry is H, or a protecting group, whereby Rx and Ry together, or Y and Rx together, may form a cyclic protecting group; Hal is a halogen; Xa is a leaving group such as halogen, or a sulfonyl ester, preferably Cl, Br, I, OTs, OTf, or ONf;

M is —B(OH)₂, —Sn(1-4C-alkyl)₃, —ZnCl, —ZnBr, —ZnI, or,

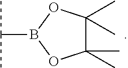

Compounds of general formula (I*) may be prepared from compounds of general formula (II). Rx may optionally be R6, or a protecting group, or other such precursor which requires further manipulation. For example, Rx in compounds of general formula (II) may be a protecting group such as the Boc group, —CO(OtBu), or Rx and Ry, together with the nitrogen to which they are attached, form a cyclic protecting group such as a phthalimide. Preparation of compounds of general formula (I*) may thus be accomplished by use of an appropriate deprotection reaction, such as in the case of a Boc group, acidic reaction conditions, for example, with a solution of 4M hydrogen chloride in dioxane, in an appropriate solvent, such as for example DCM and methanol, at ambient temperature. Further conditions to deprotect the Boc group, or further protecting groups that may be suitable for use in blocking the amino functionality in compounds of general formula (II), including their synthesis and deprotection, are found, for example, in T. W. Greene, Protective Groups in Organic Synthesis, John Wiley & Sons, 1999, 3rd Ed., or in P. Kocienski, Protecting Groups, Thieme Medical Publishers, 2000. Similarly, when Ry is not H, then Ry is a protecting group, such as for example when Rx and Ry together form a cyclic protecting group such as for example a phthalamide.

Compounds of general formula (II) may be prepared by reacting a compound of general formula (III) with a compound of general formula (IV), for example by a transition metal catalysed C—C bond formation. This transition metal catalysed C—C bond formation reaction can, for example, be achieved if M has the meaning of,

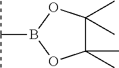

and Xa is Cl, in a suitable solvent such as THF, NMP, DMF, DME, dioxane or mixtures of the above, in the presence of a suitable base, such as aqueous sodium carbonate or potassium carbonate solution, at a suitable temperature, such as from 60° C. to 120° C. and by employing a suitable metal catalyst, such as a palladium catalyst, for example 1,1′-bis(diphenylphosphino)ferrocene]dichloropalladium(II) [Pd(dppf)Cl₂], bis(tri-tert.-butylphosphin)palladium(0) [Pd(PtBu₃)₂], or Pd(PPh₃)₄.

Compounds of general formula (IV) may be prepared from compounds of general formula (V) using known methods, for example, if M has the meaning of

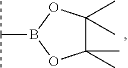

by way of a palladium catalysed borylation reaction, using a suitable metal complex such as a palladium complex formed in situ from a suitable palladium salt and a suitable phosphine ligand, for example, PdCl₂(CH₃CN)₂ and SPhos (CAS 657408-07-6), or a preformed palladium complex such as a suitable boron reagent, such as pinacol borane, or bis(pinacolato)diboron (CAS 73183-34-3), a suitable solvent, such as dioxane, DMSO, or THF, and elevated temperatures, such as up to the boiling point of the solvent, preferably 80-120° C. An analogue procedure for the palladium catalysed borylation of aryl halides using pinacol borane is reported by Buchwald et al in J. Org. Chem. 2008, p5589. Alternatively, borylation may be achieved by halogen-metal exchange, followed by quenching of the anion with a suitable borate ester. For example, compounds of general formula (IV) may be reacted with 2 Eq of sec-butyl lithium or n-butyl lithium in a suitable solvent such as THF, at suitable temperature, such as from −78° C. to −20° C., preferably from −78° C. to −50° C., followed by reaction with methyl pinacol borate or isopropyl pinacol borate. Analogous procedures are known in the literature, such as in EP1870099.

Compounds of general formula (V) and (VI) are either commercially available, may be prepared using the methods described below, may be prepared using known methods, or may be prepared by analogous methods to those known by the person skilled in the art.

One aspect of the invention is the reaction of compounds of general formulae (III) and (IV) to form a compound of general formula (II) as well as deprotection of the compound of general formula (II) to form a compound of general formula (I*).

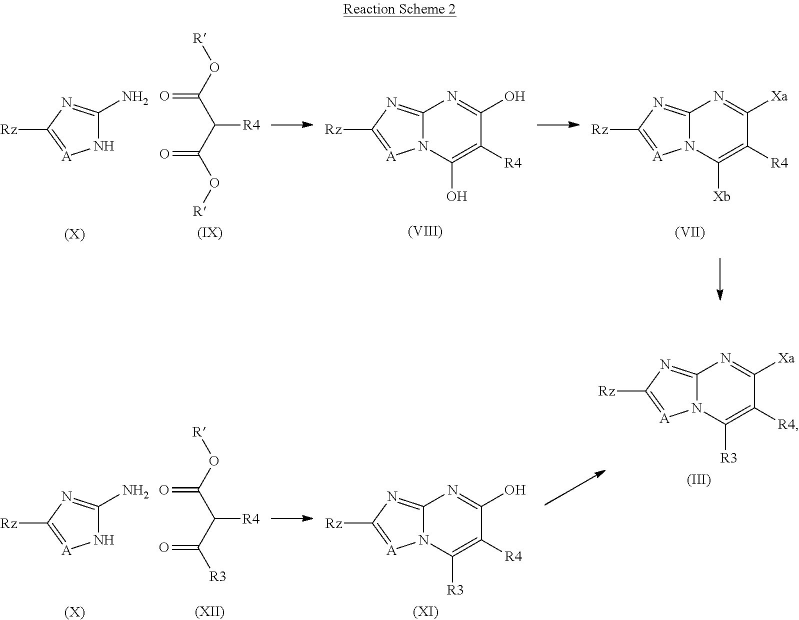

Compounds of general formula (III) may be prepared according to reaction scheme 2 wherein A, R1, R3 and R4 have the meanings defined above; Xa and Xb are halogen; Rz has the meaning R1 or R12 as defined above and R′ is 1-4C-alkyl.

Compounds of general formula (III) in which R3 is hydrogen, can be obtained from a compound of general formula (VII). This reaction can for example be achieved by reaction with a suitable reducing agent, such as zinc or Zinc/Copper pair in a suitable solvent such as mixture of THF, methanol and water at suitable temperature, such as from 0° C. to 80° C., preferable ambient temperature.

Alternatively this reaction can for example be achieved by reaction with zinc in a mixture of ammonia solution, dichloromethane and brine at suitable temperatures such as from 0° C. to 80° C., preferably from 0° C. to ambient temperature.

Alternatively, compounds of formula (III), wherein R3 is NR15R16, can be obtained by reaction of a corresponding compound of formula (VII) with the respective corresponding amino compound, HNR15R16, for example NH₂CH₃, in a suitable solvent such as THF, NMP or DMF, at a suitable temperature, such as 50° C. to the boiling point of the solvent.

Alternatively, compounds of general formula (III) in which R3 has the meaning of 1-4C-alkyl or 3-7-cycloalkyl, can for example be prepared from corresponding compounds of formula (XI) by treatment with a suitable halogenation reagent, such as phosphorus oxychloride in the case that Xa has the meaning of Cl, or phosphorus tribromide or phosphorus oxybromide in the case that Xa has the meaning of Br.

Compounds of general formula (VII) can be synthesized from corresponding compounds of formula (VIII) with a suitable halogenation reagent, for example, phosphorus oxychloride, phosphorus tribromide, phosphorus oxybromide.

Compounds of general formula (VIII), can be prepared with a condensation of the corresponding amino heterocycle of formula (X) and the malonate esters of formula (IX). This reaction can, for example, be accomplished in DMF at elevated temperatures of from 80 to 200° C. and by employing a base such as diaza(1,3)bicyclo[5.4.0]undecane (DBU) or tributylamine.

Compounds of general formula (XI), wherein R3 is 1-4C-alkyl or 3-7-cycloalkyl can be prepared, for example, with a condensation of the corresponding amino heterocycle of formula (X) and the beta ketoesters of formula (XII). This reaction can, for example, be accomplished in DMF at elevated temperatures of from 80 to 200° C. and by employing a base such as DBU or tributylamine.

Compounds of formulae (X), (XI), or (XII) are either commercially available, may be prepared using the methods described below, may be prepared using known methods, or may be prepared by analogous methods to those known by the person skilled in the art.

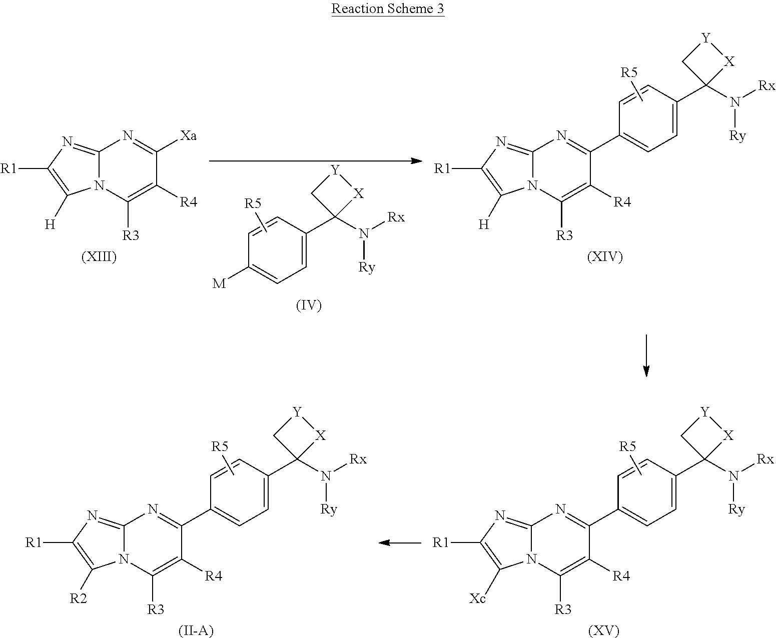

Compounds of general formula (I-A), may be prepared from compounds of general formula (II-A) as described above for compounds of general formula (I*) [reaction scheme 1].

Compounds of general formula (II-A) may be prepared according to reaction scheme 3, wherein A is C—R2, whereby R2 is not H, X, Y, R1, R3, R4, R5 and R6 have the meanings defined above, whereby Rx has the meaning of R6 and may also be a protecting group; Ry is H, or a protecting group, whereby Rx and Ry together, or Y and Rx together, may form a cyclic protecting group; Xa and Xc are halogen, preferably Cl, Br, or I; M is —B(OH)₂, —Sn(1-4C-alkyl)₃, —ZnCl, —ZnBr, —ZnI, or,

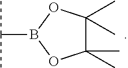

According to reaction scheme 3, compounds of general formula (II-A), may be prepared from compounds of general formula (XV) by known methods, such as for example transition metal catalysed reactions. For example, compounds for which R2 has the meaning of 1-3C-alkyl, 3-7C-cycloalkyl, —CN, aryl, heteroaryl, 2-4C-alkenyl and 2-4C-alkynyl can be obtained from compounds of general formula (XV), wherein Xc has the meaning of a halogen, by reaction with a metal organic reagent, such as, but not limited to 1-3C-alkyl-B(OH)₂, 1-3C-alkyl-ZnCl, 1-3C-alkyl-ZnBr, 1-3C-alkyl-ZnI, 3-7C-cycloalkyl-B(OH)₂, 3-7C-cycloalkyl-ZnCl, 3-7C-cycloalkyl-ZnBr, 3-7C-cycloalkyl-ZnI, aryl-B(OH)₂, aryl-ZnCl, aryl-ZnBr, aryl-ZnI, heteroaryl-B(OH)₂, heteroaryl-ZnCl, heteroaryl-ZnBr, aryl-ZnI, 2-4C-alkenyl-B(OH)₂, 2-4C-alkenyl-ZnCl, 2-4C-alkenyl-ZnBr, 2-4C-alkenyl-ZnI, 2-4C-alkynyl-B(OH)₂, 2-4C-alkynyl-ZnCl, 2-4C-alkynyl-ZnBr, 2-4C-alkynyl-ZnI, Zn(CN)₂ and 2-4C-alkynyls with a terminal triple bond. For example in the case of aryl-B(OH)₂ by employing a suitable metal catalyst such as 1,1bis(diphenylphosphino)ferrocenedichloropalladium(II), in the presence of a suitable base, such as aqueous sodium carbonate, in a suitable solvent such as dioxane, at a suitable temperature, such as from 80° C. to the boiling points of the solvent.

Compounds of general formula (XV) may be prepared from compounds of general formula (XIV) by way of known halogenation reaction. For example, in the case of Xc is Br, bromination with a suitable brominating agent such as NBS may be performed. Compounds of general formula (XIV) may be prepared from compounds of general formula (XIII) by reaction with a compound of general formula (IV) in analogy to methods described above.

In a further variant, compounds of general formula (II) may be prepared by changing the order of the steps so that halogenation of (XIII), introduction of R2 and finally reaction with (IV) is performed. In another further variant, compounds of general formula (II) may be prepared by changing the order of the steps so that halogenation of (XIII), reaction with (IV) and finally introduction of R2 is performed.

One preferred aspect of the invention is the process for the preparation of the compounds of claims 1-6 according to reaction schemes 1 to 3, specifically according to the examples.

### Commercial Utility

The compounds of formula (I) and the stereoisomers of the compounds of formula (I) according to the invention are hereinafter referred to as the compounds of the invention. In particular, the compounds of the invention are pharmaceutically acceptable. The compounds according to the invention have valuable pharmaceutical properties, which make them commercially utilizable. In particular, they inhibit the Pi3K/Akt pathway and exhibit cellular activity. They are expected to be commercially applicable in the therapy of diseases (e.g. diseases dependent on overactivated Pi3K/Akt). An abnormal activation of the PI3K/AKT pathway is an essential step towards the initiation and maintenance of human tumors and thus its inhibition, for example with AKT inhibitors, is understood to be a valid approach for treatment of human tumors. For a recent review see Garcia-Echeverria et al (Oncogene, 2008, 27, 551-5526).

Cellular activity and analogous terms in the present invention is used as known to persons skilled in the art, as an example, inhibition of phosphorylation, inhibition of cellular proliferation, induction of apoptosis or chemosensitization.

Chemosensitization and analogous terms in the present invention is used as known to persons skilled in the art. These stimuli include, for example, effectors of death receptor and survival pathways as well as cytotoxic/chemotherapeutic and targeted agents and finally radiation therapy. Induction of apoptosis and analogous terms according to the present invention are used to identify a compound which excecutes programmed cell death in cells contacted with that compound or in combination with other compounds routinely used for therapy.

Apoptosis in the present invention is used as known to persons skilled in the art. Induction of apoptosis in cells contacted with the compound of this invention might not necessarily be coupled with inhibition of cell proliferation. Preferably, the inhibition of proliferation and/or induction of apoptosis are specific to cells with aberrant cell growth.

Furthermore, the compounds according to the present invention inhibit protein kinase activity in cells and tissues, causing a shift towards dephosphorylated substrate proteins and as functional consequence, for example the induction of apoptosis, cell cycle arrest and/or sensitization towards chemotherapeutic and target-specific cancer drugs. In a preferred embodiment, inhibition of the Pi3K/Akt pathway induces cellular effects as mentioned herein, alone, or in combination with standard cytotoxic or targeted cancer drugs.

In addition inhibition of AKT signaling pathway was found to inhibit retinal neovascularisation in the oxygene induced retinopathy model as well as a potential therapeutic use of a AKT inhibition on choroidal neovascularisation was shown (Wang et al., Acta Histochem. Cytochem. 44(2): 103-111, 2011; Yang et al., Investigative Ophthalmology & Visual Science (IOVS), April 2009, Vol. 50, No. 4) These results lead to the conclusion that AKT inhibition could provide a useful therapy for ocular diseases associated with ocular neovascularisation like e.g. AMD, MD und diabetic retinopathy.

Thus one embodiment of the invention includes methods of treatment of ocular diseases associated with ocular neovasculariation especially AMD, MD und diabetic retinopathy comprising administering a compound of general formula (I) as well as the use of those compounds for the treatment of said diseases.

Compounds according to the present invention exhibit anti-proliferative and/or pro-apoptotic and/or chemosensitizing properties. Accordingly, the compounds of the present invention are useful for the treatment of hyperproliferative disorders, in particular cancer. Therefore the compounds of the present invention are useful to induce an anti-proliferative and/or pro-apoptotic and/or chemosensitizing effect in mammals, such as humans, suffering from a hyperproliferative disorders, like cancer.

The invention further relates to a compound according to the invention or a pharmaceutically acceptable salt thereof, for the treatment and/or prophylaxis, preferably treatment of (hyper)proliferative diseases and/or disorders responsive to induction of apoptosis, which include benign neoplasia and malignant neoplasia, especially malignant neoplasia, including cancer and the tumor types as disclosed below.

Compounds according to the present invention exhibit anti-proliferative and/or pro-apoptotic properties in mammals such as humans due to inhibition of metabolic activity of cancer cells which are able to survive despite of unfavourable growth conditions such as glucose depletion, hypoxia or other chemo stress.

Thus, the compounds according to the present invention are useful for treating, ameliorating or preventing diseases of benign or malignant behaviour as described herein, such as e.g. for inhibiting cellular neoplasia.

Neoplasia in the present invention is used as known to persons skilled in the art. A benign neoplasia is described by hyperproliferation of cells, incapable of forming an aggressive, metastasizing tumor in-vivo. In contrast, a malignant neoplasia is described by cells with multiple cellular and biochemical abnormalities, capable of forming a systemic disease, for example forming tumor metastasis in distant organs.

The compounds according to the present invention can be preferably used for the treatment of malignant neoplasia. Examples of malignant neoplasia treatable with the compounds according to the present invention include solid and hematological tumors. Solid tumors can be exemplified by tumors of the breast, bladder, bone, brain, central and peripheral nervous system, colon, endocrine glands (e.g. thyroid and adrenal cortex), esophagus, endometrium, germ cells, head and neck, kidney, liver, lung, larynx and hypopharynx, mesothelioma, ovary, pancreas, prostate, rectum, renal, small intestine, soft tissue, testis, stomach, skin, ureter, vagina and vulva. Malignant neoplasias include inherited cancers exemplified by Retinoblastoma and Wilms tumor. In addition, malignant neoplasias include primary tumors in said organs and corresponding secondary tumors in distant organs (“tumor metastases”). Hematological tumors can be exemplified by aggressive and indolent forms of leukemia and lymphoma, namely non-Hodgkins disease, chronic and acute myeloid leukemia (CML/AML), acute lymphoblastic leukemia (ALL), Hodgkins disease, multiple myeloma and T-cell lymphoma. Also included are myelodysplastic syndrome, plasma cell neoplasia, paraneoplastic syndromes, and cancers of unknown primary site as well as AIDS related malignancies.

The invention further includes as a preferred embodiment methods for treatment of breast cancer.

It is noted that a malignant neoplasia does not necessarily require the formation of metastases in distant organs. Certain tumors exert devastating effects on the primary organ itself through their aggressive growth properties. These can lead to the destruction of the tissue and organ structure finally resulting in failure of the assigned organ function and death.

Drug resistance is of particular importance for the frequent failure of standard cancer therapeutics. This drug resistance is caused by various cellular and molecular mechanisms. One aspect of drug resistance is caused by constitutive activation of anti-apoptotic survival signals with PKB/Akt as a key signalling kinase. Inhibition of the Pi3K/Akt pathway leads to a resensitization towards standard chemotherapeutic or target specific cancer therapeutics. As a consequence, the commercial applicability of the compounds according to the present invention is not limited to 1^{st} line treatment of cancer patients. In a preferred embodiment, cancer patients with resistance to cancer chemotherapeutics or target specific anti-cancer drugs are also amenable for treatment with these compounds for e.g. 2^{nd} or 3^{rd} line treatment cycles. In particular, the compounds according to the present invention might be used in combination with standard chemotherapeutic or targeted drugs to resensitize tumors towards these agents.

Compounds according to the present invention are suitable for treatment, prevention or amelioration of the diseases of benign and malignant behavior as described above, such as e.g. benign or malignant neoplasia, particularly cancer, especially a cancer that is sensitive to Pi3K/Akt pathway inhibition.

The present invention further includes a method for treating, preventing or ameliorating mammals, including humans, preferably treating mammals, including humans, which are suffering from one of the abovementioned conditions, illnesses, disorders or diseases. The method is characterized in that a pharmacologically active and therapeutically effective and tolerable amount of one or more of the compounds according to the present invention is administered to the subject in need of such treatment.

The present invention further includes a method for treating, preventing or ameliorating diseases responsive to inhibition of the Pi3K/Akt pathway, in a mammal, including human, preferably treating diseases responsive to inhibition of the Pi3K/Akt pathway, in a mammal, including human, comprising administering a pharmacologically active and therapeutically effective and tolerable amount of one or more of the compounds according to the present invention to said mammal.

The present invention further includes a method for inhibiting protein kinase activity in cells comprising administering a pharmacologically active and therapeutically effective and tolerable amount of one or more of the compounds according to the present invention to a patient in need of such therapy.

The present invention further includes a method for treating hyperproliferative diseases of benign or malignant behaviour and/or disorders responsive to induction of apoptosis, such as e.g. cancer, particularly any of those cancer diseases described above, in a mammal, comprising administering a pharmacologically active and therapeutically effective and tolerable amount of one or more of the compounds according to the present invention to said mammal.

The present invention further includes a method for inhibiting cellular hyperproliferation or arresting aberrant cell growth in a mammal, comprising administering a pharmacologically active and therapeutically effective and tolerable amount of one or more of the compounds according to the present invention to said mammal.

The present invention further includes a method for inducing apoptosis in the therapy of benign or malignant neoplasia, particularly cancer, comprising administering a pharmacologically active and therapeutically effective and tolerable amount of one or more of the compounds according to the present invention to a subject in need of such therapy.

The present invention further includes a method for inhibiting protein kinase activity in cells comprising administering a pharmacologically active and therapeutically effective and tolerable amount of one or more of the compounds according to the present invention to a patient in need of such therapy.

The present invention further includes a method for sensitizing towards chemotherapeutic or target-specific anti-cancer agents in a mammal, comprising administering a pharmacologically active and therapeutically effective and tolerable amount of one or more of the compounds according to the present invention to said mammal.

The present invention further includes a method for treating benign and/or malignant neoplasia, especially malignant neoplasia, particularly cancer, in a mammal, including human, comprising administering a pharmacologically active and therapeutically effective and tolerable amount of one or more of the compounds according to the present invention to said mammal.

The present invention further includes a method for treating solid and hematological tumors, whereby solid tumors can be exemplified by tumors of the breast, bladder, bone, brain, central and peripheral nervous system, colon, endocrine glands (e.g. thyroid and adrenal cortex), esophagus, endometrium, germ cells, head and neck, kidney, liver, lung, larynx and hypopharynx, mesothelioma, ovary, pancreas, prostate, rectum, renal, small intestine, soft tissue, testis, stomach, skin, ureter, vagina and vulva. Malignant neoplasias include inherited cancers exemplified by Retinoblastoma and Wilms tumor. In addition, malignant neoplasias include primary tumors in said organs and corresponding secondary tumors in distant organs (“tumor metastases”). and hematological tumors can be exemplified by aggressive and indolent forms of leukemia and lymphoma, namely non-Hodgkins disease, chronic and acute myeloid leukemia (CML/AML), acute lymphoblastic leukemia (ALL), Hodgkins disease, multiple myeloma and T-cell lymphoma. Also included are myelodysplastic syndrome, plasma cell neoplasia, paraneoplastic syndromes, and cancers of unknown primary site as well as AIDS related malignancies.

The present invention further relates to the use of the compounds for the pro-duction of pharmaceutical compositions, which are employed for the treatment, prophylaxis, and/or amelioration of one or more of the illnesses mentioned, preferably for the treatment of one or more of the illnesses mentioned.

The present invention further relates to the use of the compounds for the manufacture of pharmaceutical compositions for treating, preventing or ameliorating, preferably treating hyperproliferative diseases and/or disorders responsive to the induction of apoptosis, such as e.g. benign or malignant neoplasia, especially malignant neoplasia, in particular cancer, especially those cancer diseases and tumor types mentioned above.

The present invention further relates to the use of the compounds according to this invention for the production of pharmaceutical compositions for treating, preventing or ameliorating, preferably treating benign or malignant neoplasia, especially malignant neoplasia, particularly cancer, such as e.g. any of those cancer diseases and tumor types described above.

The invention further relates to a compound according to the invention or a pharmaceutically acceptable salt thereof, for the treatment and/or prophylaxis, preferably treatment of (hyper)proliferative diseases and/or disorders responsive to induction of apoptosis, which include benign neoplasia and malignant neoplasia, including cancer.

The invention further related to the use of a compound according to the invention or a pharmaceutically acceptable salt thereof, for the production of a pharmaceutical composition for the treatment, prevention or amelioration of a disease mediated by a dysregulated function of a single protein kinase or multiple protein kinases and/or disorders responsive to the induction of apoptosis.

The invention further relates to a pharmaceutical composition, comprising a compound according to the invention or a pharmaceutically acceptable salt thereof, for the treatment and/or prophylaxis, preferably treatment of (hyper)proliferative diseases and/or disorders responsive to induction of apoptosis, which include benign neoplasia and malignant neoplasia, including cancer.

The present invention further relates to the use of compounds and pharmaceutically acceptable salts according to the present invention for the manufacture of pharmaceutical compositions, which can be used for sensitizing towards chemotherapeutic and/or target specific anti-cancer agents.

The present invention further relates to the use of compounds according to the present invention for the manufacture of pharmaceutical compositions, which can be used for sensitizing towards radiation therapy of those diseases mentioned herein, particularly cancer.

The present invention further relates to the use of the compounds according to the present invention for the manufacture of pharmaceutical compositions, which can be used in the treatment of diseases sensitive to protein kinase inhibitor therapy and different to cellular neoplasia. These non-malignant diseases include, but are not limited to benign prostate hyperplasia, neurofibromatosis, dermatoses, and myelodysplastic syndromes.

The present invention further relates to pharmaceutical compositions comprising one or more of the compounds according to this invention and a pharmaceutically acceptable carrier or diluent.

The present invention further relates to pharmaceutical compositions comprising one or more of the compounds according to this invention and pharmaceutically acceptable auxiliaries and/or excipients.

The pharmaceutical compositions according to this invention are prepared by processes, which are known per se and familiar to the person skilled in the art. As pharmaceutical compositions, the compounds of the invention (=active compounds) are either employed as such, or preferably in combination with suitable pharmaceutical auxiliaries and/or excipients, e.g. in the form of tablets, coated tablets, dragees, pills, cachets, granules, capsules, caplets, suppositories, patches (e.g. as TTS), emulsions (such as e.g. micro-emulsions or lipid emulsions), suspensions (such as e.g. nano suspensions), gels, solubilisates or solutions (e.g. sterile solutions), or encapsuled in liposomes or as beta-cyclodextrine or beta-cyclodextrin derivative inclusion complexes or the like, the active compound con-tent advantageously being between 0.1 and 95% and where, by the appropriate choice of the auxiliaries and/or excipients, a pharmaceutical administration form (e.g. a delayed release form or an enteric form) exactly suited to the active compound and/or to the desired onset of action can be achieved.

The person skilled in the art is familiar with auxiliaries, vehicles, excipients, diluents, carriers or adjuvants which are suitable for the desired pharmaceutical formulations, preparations or compositions on account of his/her expert knowledge. In addition to solvents, gel formers, ointment bases and other active compound excipients, for example antioxidants, dispersants, emulsifiers, preser-vatives, solubilizers (such as e.g. polyoxyethylenglyceroltriricinoleat 35, PEG 400, Tween 80, Captisol, Solutol HS15 or the like), colorants, complexing agents, permeation promoters, stabilizers, fillers, binders, thickeners, disintegrating agents, buffers, pH regulators (e.g. to obtain neutral, alkaline or acidic formulations), polymers, lubricants, coating agents, propellants, tonicity adjusting agents, surfactants, flavorings, sweeteners or dyes, can be used.

In particular, auxiliaries and/or excipients of a type appropriate to the desired formulation and the desired mode of administration are used.

The administration of the compounds, pharmaceutical compositions or combinations according to the invention may be performed in any of the generally accepted modes of administration available in the art. Illustrative examples of suitable modes of administration include intravenous, oral, nasal, parenteral, topical, transdermal and rectal delivery. Oral and intravenous deliveries are preferred.

Generally, the pharmaceutical compositions according to the invention can be administered such that the dose of the active compound is in the range customary for Pi3K/Akt pathway inhibitors. In particular, a dose in the range of from 0.01 to 4000 mg of the active compound per day is preferred for an average adult patient having a body weight of 70 kg. In this respect, it is to be noted that the dose is dependent, for example, on the specific compound used, the species treated, age, body weight, general health, sex and diet of the subject treated, mode and time of administration, rate of excretion, severity of the disease to be treated and drug combination.

The pharmaceutical composition can be administered in a single dose per day or in multiple subdoses, for example, 2 to 4 doses per day. A single dose unit of the pharmaceutical composition can contain e.g. from 0.01 mg to 4000 mg, preferably 0.1 mg to 2000 mg, more preferably 0.5 to 1500 mg, most preferably 1 to 500 mg, of the active compound. Furthermore, the pharmaceutical composition can be adapted to weekly, monthly or even more infrequent administration, for example by using an implant, e.g. a subcutaneous or intramuscular implant, by using the active compound in form of a sparingly soluble salt or by using the active compound coupled to a polymer.

The present invention further relates to combinations comprising one or more first active ingredients selected from the compounds of the invention and one or more second active ingredients selected from chemotherapeutic anti-cancer agents and target-specific anti-cancer agents e.g. for treating, preventing or ameliorating diseases responsive or sensitive to inhibition of the Pi3K/Akt pathway, such as hyperproliferative diseases of benign or malignant behaviour and/or disorders responsive to the induction of apoptosis, particularly cancer, such as e.g. any of those cancer diseases described above.

The invention further relates to the use of a pharmaceutical composition comprising one or more of the compounds according to this invention as sole active ingredient(s) and a pharmaceutically acceptable carrier or diluent in the manufacture of pharmaceutical products for the treatment and/or prophylaxis of the illnesses mentioned above.

Depending upon the particular disease, to be treated or prevented, additional therapeutic active agents, which are normally administered to treat or prevent that disease, may optionally be coadministered with the compounds according to this invention. As used herein, additional therapeutic agents that are normally administered to treat or prevent a particular disease are known as appropriate for the disease being treated.

The anti-cancer agents mentioned herein above as combination partners of the compounds according to this invention are meant to include pharmaceutically acceptable derivatives thereof, such as e.g. their pharmaceutically acceptable salts.

The person skilled in the art is aware of the total daily dosage(s) and administration form(s) of the additional therapeutic agent(s) coadministered. Said total daily dosage(s) can vary within a wide range depending from the agent combined.

In practicing the present invention, the compounds according to this invention may be administered in combination therapy separately, sequentially, simultaneously, concurrently or chronologically staggered (such as e.g. as combined unit dosage forms, as separate unit dosage forms, as adjacent discrete unit dosage forms, as fixed or non-fixed combinations, as kit-of-parts or as admixtures) with one or more standard therapeutics (chemotherapeutic and/or target specific anti-cancer agents), in particular art-known anti-cancer agents, such as any of e.g. those mentioned above.

In this context, the present invention further relates to a combination comprising a first active ingredient, which is at least one compound according to this invention, and a second active ingredient, which is at least one art-known anti-cancer agent, such as e.g. one or more of those mentioned herein above, for separate, sequential, simultaneous, concurrent or chronologically staggered use in therapy, such as e.g. in therapy of any of those diseases mentioned herein.

The present invention further relates to a pharmaceutical composition comprising a first active ingredient, which is at least one compound according to this invention, and a second active ingredient, which is at least one art-known anti-cancer agent, such as e.g. one or more of those mentioned herein above, and, optionally, a pharmaceutically acceptable carrier or diluent, for separate, sequential, simultaneous, concurrent or chronologically staggered use in therapy.

The present invention further relates to a combination product comprising

a.) at least one compound according to this invention formulated with a pharmaceutically acceptable carrier or diluent, and
b.) at least one art-known anti-cancer agent, such as e.g. one or more of those mentioned herein above, formulated with a pharmaceutically acceptable carrier or diluent.

The present invention further relates to a kit-of-parts comprising a preparation of a first active ingredient, which is a compound according to this invention, and a pharmaceutically acceptable carrier or diluent; a preparation of a second active ingredient, which is an art-known anti-cancer agent, such as one of those mentioned above, and a pharmaceutically acceptable carrier or diluent; for simul-taneous, concurrent, sequential, separate or chronologically staggered use in therapy. Optionally, said kit comprises instructions for its use in therapy, e.g. to treat hyperproliferative diseases and diseases responsive or sensitive to inhibition of the Pi3K/Akt pathway, such as e.g. benign or malignant neoplasia, particularly cancer, more precisely, any of those cancer diseases described above.

The present invention further relates to a combined preparation comprising at least one compound according to this invention and at least one art-known anti-cancer agent for simultaneous, concurrent, sequential or separate administration.

The present invention further relates to combinations, compositions, formulations, preparations or kits according to the present invention having Pi3K/Akt pathway inhibitory activity.

In addition, the present invention further relates to a method for treating in combination therapy hyperproliferative diseases and/or disorders responsive to the induction of apoptosis, such as e.g. cancer, in a patient comprising administering a combination, composition, formulation, preparation or kit as described herein to said patient in need thereof.

In addition, the present invention further relates to a method for treating hyperproliferative diseases of benign or malignant behaviour and/or disorders responsive to the induction of apoptosis, such as e.g. cancer, in a patient comprising administering in combination therapy separately, simultaneously, concurrently, sequentially or chronologically staggered a pharmaceutically active and therapeutically effective and tolerable amount of a pharmaceutical composition, which comprises a compound according to this invention and a pharmaceutically acceptable carrier or diluent, and a pharmaceutically active and therapeutically effective and tolerable amount of one or more art-known anti-cancer agents, such as e.g. one or more of those mentioned herein, to said patient in need thereof.

In further addition, the present invention relates to a method for treating, preventing or ameliorating hyperproliferative diseases and/or disorders responsive to induction of apoptosis, such as e.g. benign or malignant neoplasia, e.g. cancer, particularly any of those cancer diseases mentioned herein, in a patient comprising administering separately, simultaneously, concurrently, sequentially or chronologically staggered to said patient in need thereof an amount of a first active compound, which is a compound according to the present invention, and an amount of at least one second active compound, said at least one second active compound being a standard therapeutic agent, particularly at least one art-known anti-cancer agent, such as e.g. one or more of those chemotherapeutic and target-specific anti-cancer agents mentioned herein, wherein the amounts of the first active compound and said second active compound result in a therapeutic effect.

In yet further addition, the present invention relates to a method for treating, preventing or ameliorating, especially treating hyperproliferative diseases and/or disorders responsive to induction of apoptosis, such as e.g. benign or malignant neoplasia, especially malignant neoplasia, e.g. cancer, particularly any of those cancer diseases and tumor types mentioned herein, in a patient comprising administering a combination according to the present invention.

In addition, the present invention further relates to the use of a composition, combination, formulation, preparation or kit according to this invention in the manufacture of a pharmaceutical product, such as e.g. a commercial package or a medicament, for treating, preventing or ameliorating, especially treating hyperproliferative diseases, and/or disorders responsive to the induction of apoptosis, such as e.g. malignant or benign neoplasia, especially malignant neoplasia, such as e.g. cancer, particularly those diseases and tumor types mentioned herein.

The present invention further relates to a commercial package comprising one or more compounds of the present invention together with instructions for simultaneous, concurrent, sequential or separate use with one or more chemotherapeutic and/or target specific anti-cancer agents, such as e.g. any of those mentioned herein.

The present invention further relates to a commercial package consisting essentially of one or more compounds of the present invention as sole active ingredient together with instructions for simultaneous, concurrent, sequential or separate use with one or more chemotherapeutic and/or target specific anti-cancer agents, such as e.g. any of those mentioned herein.

The present invention further relates to a commercial package comprising one or more chemotherapeutic and/or target specific anti-cancer agents, such as e.g. any of those mentioned herein, together with instructions for simultaneous, concurrent, sequential or separate use with one or more compounds according to the present invention.

The compositions, combinations, preparations, formulations, kits or packages mentioned in the context of the combination therapy according to this invention may also include more than one of the compounds according to this invention and/or more than one of the art-known anti-cancer agents mentioned.

The first and second active ingredient of a combination or kit-of-parts according to this invention may be provided as separate formulations (i.e. independently of one another), which are subsequently brought together for simultaneous, concurrent, sequential, separate or chronologically staggered use in combination therapy; or packaged and presented together as separate components of a combination pack for simultaneous, concurrent, sequential, separate or chronologically staggered use in combination therapy.

The type of pharmaceutical formulation of the first and second active ingredient of a combination or kit-of-parts according to this invention can be according, i.e. both ingredients are formulated in separate tablets or capsules, or can be different, i.e. suited for different administration forms, such as e.g. one active ingredient is formulated as tablet or capsule and the other is formulated for e.g. intravenous administration.

The amounts of the first and second active ingredients of the combinations, compositions or kits according to this invention may together comprise a therapeutically effective amount for the treatment, prophylaxis or amelioration of a hyperproliferative diseases and/or a disorder responsive to the induction of apoptosis, particularly one of those diseases mentioned herein, such as e.g. malignant or benign neoplasia, especially malignant neoplasia, e.g. cancer, like any of those cancer diseases and tumor types mentioned herein.

In addition, compounds according to the present invention can be used in the pre- or post-surgical treatment of cancer.

In further addition, compounds of the present invention can be used in combination with radiation therapy.

As will be appreciated by persons skilled in the art, the invention is not limited to the particular embodiments described herein, but covers all modifications of said embodiments that are within the spirit and scope of the invention as defined by the appended claims.

The following examples illustrate the invention in greater detail, without restricting it. Further compounds according to the invention, of which the preparation is not explicitly described, can be prepared in an analogous way.

The compounds, which are mentioned in the examples and the salts thereof represent preferred embodiments of the invention as well as a claim covering all subcombinations of the residues of the compound of formula (I) as disclosed by the specific examples.

The term “according to” within the experimental section is used in the sense that the procedure referred to is to be used “analogously to”.

### Experimental Part

The following table lists the abbreviations used in this paragraph and in the Intermediate Examples and Examples section as far as they are not explained within the text body. NMR peak forms are stated as they appear in the spectra, possible higher order effects have not been considered. Chemical names were generated using AutoNom2000 as implemented in MDL ISIS Draw. In some cases generally accepted names of commercially available reagents were used in place of AutoNom2000 generated names.

| Abbreviation | Meaning |
|---|---|
| Boc | t-Butoxycarbonyl |
| br | broad |
| CI | chemical ionisation |
| d | doublet |
| dd | doublet of doublet |
| DAD | diode array detector |
| DBU | 1,5-diazabicyclo(5.4.0)undec-5-ene |
| DCM | dichloromethane |
| DIP | diisopropylether |
| EtOAc | ethyl acetate |
| Eq. | equivalent |
| ESI | electrospray (ES) ionisation |
| HPLC | high performance liquid chromatography |
| LC-MS | liquid chromatography mass spectrometry |
| m | multiplet |
| MS | mass spectrometry |
| n-BuLi | n-Butyllithium |
| NMP | N-methyl pyrrolidone |
| NMR | nuclear magnetic resonance spectroscopy: |
| | chemical shifts (δ) are given in ppm. |
| ONf | nonafluorobutanesulfonate |
| OTf | trifluoromethanesulfonate |
| OTs | tosylate |
| Pd(dppf)Cl₂ | 1,1′ |
| | bis(diphenylphosphino)ferrocene]dichloropalladium |
| | (II) |
| Pd(PtBu₃)₂ | bis (tri-tert.-butylphosphin)palladium(0) |
| | [Pd(PtBu₃)₂], |
| q | quartet |
| r.t. or rt | room temperature |
| RT | retention time (as measured either with HPLC or |
| | UPLC) in minutes |
| s | singlet |
| t | triplet |
| THF | tetrahydrofuran |
| UPLC | ultra performance liquid chromatography |

Other abbreviations have their meanings customary per se to the skilled person. The various aspects of the invention described in this application are illustrated by the following examples which are not meant to limit the invention in any way.

### EXAMPLES

### UPLC-MS Standard Procedure

Analytical UPLC-MS was performed as described below. Method A was used unless stated otherwise. The masses (m/z) are reported from the positive mode electrospray ionisation unless the negative mode is indicated (ES−).

### UPLC-MS Method A

Instrument: Waters Acquity UPLC-MS SQD 3001; Column: Acquity UPLC BEH C18 1.7 50×2.1 mm; Eluent A: water+0.1% formic acid or 0.2% ammonia, Eluent B: acetonitrile; Gradient: 0-1.6 min 1-99% B, 1.6-2.0 min 99% B; Flow rate 0.8 ml/min; Temperature: 60° C.; Injection: 2 μl; DAD scan: 210-400 nm.

UPLC-MS Method B Instrument: Waters Acquity UPLC-MS ZQ4000; Column: Acquity UPLC BEH C18 1.7 50×2.1 mm; Eluent A: water+0.05% formic acid or 0.2% ammonia, Eluent B: acetonitrile+0.05% formic acid; Gradient: 0-1.6 min 1-99% B, 1.6-2.0 min 99% B; Flow rate 0.8 ml/min; Temperature: 60° C.; Injection: 2 μl; DAD scan: 210-400 nm.

### Intermediate Examples

### {1-[4-(4,4,5,5-tetramethyl-[1,3,2]dioxaborolan-2-yl)-phenyl]-cyclobutyl}-carbamic acid tert-butyl ester

### Step 1: [1-(4-bromo-phenyl)cyclobutyl]-carbamic acid tert-butyl ester

The free base of commercially available [1-(4-bromo-phenyl)cyclobutyl]-amine hydrochloride [CAS 1193389-40-0] (8.99 g, 34.24 mmol) was prepared as follows: (8.99 g, 34.24 mmol) of the hydrochloride salt was taken up in DCM and washed sequentially with aqueous sodium bicarbonate and water and the organic portion was dried and concentrated.

The crude amine was taken up in dry THF (120 mL) and diisopropylethylamine (17.62 mL, 102.71 mmol) under nitrogen and a solution of di-tert-butyldicarbonate (8.22 g, 37.66 mmol) in THF (20 mL) was added. The reaction was stirred at rt overnight. The mixture was partitioned between EtOAc and water and the extracted organic phase was washed with brine and concentrated in vacuo to give the title compound.

Alternatively, the title compound may also be prepared by known methods, such as those given in WO2008/70041, in particular from commercially available (4-bromo-phenyl)-acetonitrile.

Step 2: {1-[4-(4,4,5,5-tetramethyl-[1,3,2]dioxaborolan-2-yl)-phenyl]-cyclobutyl}-carbamic acid tert-butyl ester

10 g (30.7 mmol) [1-(4-Bromophenyl)-cyclobutyl]-carbamic acid tert.-butyl ester, 8.56 g (33.7 mmol) bis-(pinacolato)diboron, 750.9 mg (0.92 mmol) 1,1′-bis(diphenylphosphino)ferrocenedichloropalladium(II) and 9.03 g (92 mmol) potassium acetate are given in 180 mL THF which has been degassed for 10′. The reaction mixture is heated at reflux until the starting material has disappeared (usually two hours). At 60° C. the reaction mixture turns dark. The reaction mixture is poured on water (200 mL) and ethyl acetate (500 mL) is added. The mixture is vigorously stirred for two hours. After separation of the organic phase the aqueous phase is extracted once more with ethyl acetate (300 mL). The combined organic extracts are washed with brine and dried (sodium sulfate). After evaporation of the solvent the residue, a black oil, is purified by chromatography on silicagel (eluents: hexane/ethyl acetate). 10.4 g (90.8%) of the title compound are obtained.

MS (ES+, M+1): 374

¹H-NMR (400 MHz, d6-DMSO): δ 7.61 (d, 2H), 7.55-7.65 (br. 1H), 7.35 (d, 2H), 2.22-2.42 (m, 4H), 1.88-2.02 (m, 1H), 1.65-1.82 (m, 1H), 1.00-1.38 (m, 21H).

Alternatively, the title compound may also be prepared by known methods, such as those given in WO2008/70041.

The following Intermediates were prepared in analogy by use of the appropriate aryl bromide.

| | Analytical |
|---|---|
| Structure/Name | Data |
| 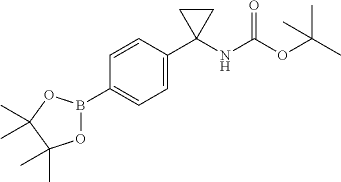 [SMILES: CC(C)(C)OC(=O)NC1(c2ccc(B3OC(C)(C)C(C)(C)O3)cc2)CC1] | |
| {1-[4-(4,4,5,5-tetramethyl- | |
| [1,3,2]dioxaborolan-2-yl)-phenyl]- | |
| cyclopropyl}-carbamic acid tert- | |
| butyl ester | |
| 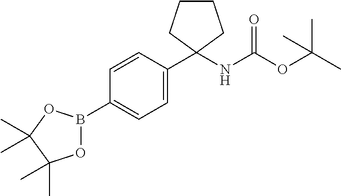 [SMILES: CC(C)(C)OC(=O)NC1(c2ccc(B3OC(C)(C)C(C)(C)O3)cc2)CCCC1] | UPLC-MS: RT = 1.60 min; m/z = 388.31 |
| {1-[4-(4,4,5,5-tetramethyl- | |
| [1,3,2]dioxaborolan-2-yl)-phenyl]- | |
| cyclopentyl}-carbamic acid tert- | |
| butyl ester | |

### Intermediate Example Int-1-0

### 7-Chloro-6-phenylimidazo[1,2-a]pyrimidine

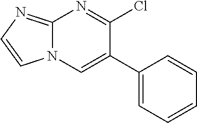 [SMILES: Clc1nc2nccn2cc1-c1ccccc1]

### Step 1: 6-phenylimidazo[1,2-a]pyrimidin-5,7-diol

26.1 g (110.4 mmol) Diethyl phenylmalonate, 20 g (110.4 mol) 2-aminoimidazole sulfate and 49 mL (331.2 mmol) DBU in 131 mL DMF are heated for 15 h at 100° C. After removal of the solvent the residue is dissolved in 200 mL water. The reaction mixture is acidified with 4M hydrochloric acid (around 100 mL) to pH 1. The precipitate is collected yielding 15.2 g (60.6%) of the desired product.

¹H-NMR (300 MHz, d6-DMSO): δ 12.3 (very br., 1H), 11.3 (very br., 1H), 7.49 (d, 1H), 7.39-7.49 (m, 2H), 7.38 (d, 1H), 7.20-7.32 (m, 2H), 7.06-7.19 (m, 1H).

### Step 2: 5,7-dichloro-6-phenylimidazo[1,2-a]pyrimidine

73.3 mL (802.7 mmol) phosphorus oxychloride are added dropwise (caution) to 15.2 g (66.9 mmol) 6-phenylimidazo[1,2-a]pyrimidin-5,7-diol. After addition of 12.7 ml (100.3 mmol) N,N-dimethylaniline the mixture is heated for two and a half hours at 100° C. After cooling the reaction mixture is poured in portions into a large amount of ice water and acidified with 4M hydrochloric acid. The formed precipitate is sucked off and dried in a vacuum furnace yielding 11 g (62.3%) of the desired product. By extraction of the filtrate an additional crop of 1 g (5.7%) has been isolated.

¹H-NMR (300 MHz, d6-DMSO): δ 8.26 (d, 1H), 8.13 (d, 1H), 7.48-7.62 (m, 3H), 7.38-7.48 (m, 2H).

### Step 3: 7-chloro-6-phenylimidazo[1,2-a]pyrimidine

4.8 g (18.2 mmol) 5,7-Dichloro-6-phenylimidazo[1,2-a]pyrimidine are dissolved in 415 mL ethanol. After addition of 295 mL Water and 177 mL THF 4.6 g (116.5 mmol) ammonium chloride and 7.6 g (116.3 mmol) zinc are added in portions. The reaction mixture is stirred over night at room temperature. An additional 1 g of zinc is added and stirring is continued for five hours. The zinc is sucked off and the organic solvents are removed by evaporation. The precipitate is sucked off and dried in a vacuum furnace yielding 2.6 g (62.3%) of the desired product which contains 20% of 6-phenylimidazo[1,2-a]pyrimidine.

¹H-NMR (300 MHz, d6-DMSO): δ 9.11 (s, 1H), 7.92 (d, 1H), 7.78 (d, 1H), 7.39-7.60 (m, 5H).

### Intermediate Example Int-1-1

### 7-Chloro-2-methyl-6-phenyl-imidazo[1,2-a]pyrimidine

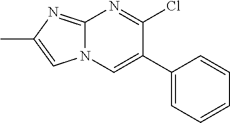 [SMILES: Cc1cn2cc(-c3ccccc3)c(Cl)nc2n1]

### Step 1: 2-methyl-6-phenyl-imidazo[1,2-a]pyrimidin-5,7-diol

18 g (135 mmol) 5-Methyl-1H-imidazol-2-ylamine are dissolved in 155 mL DMF and 31.8 g (135 mmol) diethyl phenylmalonate are added. After dropwise addition of

61.5 g (404 mmol) 1,8-diazabicyclo[5.4.0]undec-7-ene the reaction mixture is stirred for 16 h at 100° C. The DMF has been removed and the darkbrown oily residue is treated with 150 mL water (complete dissolution). 2M hydrochloric acid (250 mL) is added at room temperature until a pH of 1. After stirring for 1 h at ice bath cooling the formed crystals are collected by filtration to yield the product (10.2 g=31%), which is used without further purification.

MS (Cl, M+1): 242

¹H-NMR (300 MHz, d6-DMSO): δ 12.25 (br., 1H), 10.97 (br., 1H), 7.33-7.48 (m, 2H), 7.18-7.30 (m, 3H), 7.05-7.18 (m, 1H), 2.21 (s, 3H).

### Step 2: 5,7-dichloro-2-methyl-6-phenyl-imidazo[1,2-a]pyrimidine

10.2 g (42.3 mmol) 2-Methyl-6-phenyl-imidazo[1,2-a]pyrimidin-5,7-diol are dissolved in 48 mL (515 mmol) phosphorus oxychloride and 8.6 mL (67.6 mmol) N,N-dimethylaniline. The mixture is heated for 16 h at 100° C. Due to the presence of starting material additional 18.4 mL phosphorus oxychloride and 1.5 mL N,N-dimethylaniline are added. The heating has been continued for two days. phosphorus oxychloride is evaporated and the oily residue treated with ice-water (caution: stirring and cooling necessary due to strong development of heat). A precipitate forms. After addition of 30 mL dichloromethane the precipitate has been collected by filtration and washed with dichloromethane/water. The crude precipitate is stirred with 2N sodium hydroxide (200 mL) for one hour and collected by filtration, washed with water and dried. 10.54 g (89.6%) of the desired product are obtained.

MS (Cl, M+1): 278

¹H-NMR (300 MHz, d6DMSO): δ 7.82 (s, 1H), 7.38-7.58 (m, 5H), 2.40 (s, 3H).

### Step 3: 7-chloro-2-methyl-6-phenyl-imidazo[1,2-a]pyrimidine

8.3 g (30 mmol) 5,7-Dichloro-2-methyl-6-phenyl-imidazo[1,2-a]pyrimidine are dissolved in 14.5 mL methanol and 85 mL THF. After addition of 3.4 mL acetic acid 5.8 g (45 mmol) zinc/copper pair are added in portions and the mixture is stirred for four and a half hours at rt. The reaction mixture is filtered via a glass microfibre filter and washed with plenty of methanol. The solvent has been removed and the residue redissolved in ethyl acetate. After washing twice with brine and drying over sodium sulfate, the solvent is evaporated and the residue purified by chromatography on silica gel (eluents: dichloromethane/methanol) yielding only 260 mg of the desired compound. 2N sodium hydroxide is added to the zinc/copper slurry of the reaction until a pH of 8. Afterwards the slurry is treated four times with 300 mL ethyl acetate/methanol (1%) each. The solvent mixture is decanted each time and the extracts are combined. After evaporation of the solvents 3.1 g of the desired product are obtained. Repetition of this process yields additional 2.32 g product. Altogether 74.4% of the title compound have been obtained.

MS (Cl, M+1): 244

¹H-NMR (300 MHz, d6-DMSO): δ 8.99 (s, 1H), 7.64 (s, 1H), 7.40-7.58 (m, 5H), 2.35 (s, 3H).

### Intermediate Example Int-1-2

### 5-chloro-2-methyl-6-phenyl[1,2,4]triazolo[1,5-a]pyrimidine

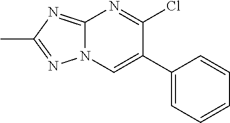 [SMILES: Cc1nc2nc(Cl)c(-c3ccccc3)cn2n1]

### Step 1: 2-methyl-6-phenyl[1,2,4]triazolo[1,5-a]pyrimidin-5,7-diol

A solution of 25.0 g 3-amino-5-methyltriazole and 66.0 mL diethyl phenylmalonate in 100 mL N,N-dibutylbutan-1-amine was stirred at 185° C. for 20 h. The reaction mixture consisted of two layers after cooling to room temperature. The top layer was removed and the lower layer was diluted with 10% w/w sodium hydroxide solution and water. The aqueous layer was extracted with diethyl ether and acidified with concentrated hydrochloric acid until precipition of the product was complete. The precipitate was collected by filtration to yield the product, which was used without further purification.

MS (M+1): 243

Characteristic ¹H NMR (200 MHz, d6-DMSO) signals: 7.4 (m, 2H); 7.3 (m, 2H); 7.2 (m, 1H); 2.4 (s, 3H) ppm.

### Step 2: 5,7-dichloro-2-methyl-6-phenyl[1,2,4]triazolo[1,5-a]pyrimidine

35.0 g 2-methyl-6-phenyl[1,2,4]triazolo[1,5-a]pyrimidin-5,7-diol was suspended in 80 mL phosphorus oxychloride. and 27.47 mL N,N-dimethylaniline were added. The mixture was stirred at 100° C. for 1 h. The excess of phosphorus oxychloride was removed and the residue was dissolved in a mixture of dichloromethane, water and ice. The organic phase was separated and the water-phase was extracted with dichloromethane. The combined organic layers were dried over sodium sulfate and the solvent was evaporated. The crude product was used without further purification.

RT=1.15 min; m/z=279 (ES+, M+1) (300 MHz, d6-DMSO): δ 7.48-7.60 (m, 3H), 7.35-7.48 (m, 2H), 2.58 (s, 3H) ppm.

### Step 3: 5-chloro-2-methyl-6-phenyl[1,2,4]triazolo[1,5-a]pyrimidine

34.5 g 5,7-dichloro-2-methyl-6-phenyl[1,2,4]triazolo[1,5-a]pyrimidine was dissolved in 500 mL dichloromethane. 500 mL brine, 250 mL 25% aqueous ammonia solution and 34.0 g zinc powder were added and the mixture was stirred at room temperature for 1 h. The reaction mixture was filtered through Celite and was washed with dichloromethane and water. The organic phase was separated and the water phase was extracted with dichloromethane. The combined dichloromethane phase was dried over sodium sulfate and the solvent was evaporated. The crude product was purified by chromatography on silica gel (dichloromethane/ethyl acetate) to yield the desired compound.

MS (M+1): 245

Characteristic ¹H NMR (300 MHz, d6-DMSO) signals: 9.45 (s, 1H), 2.6 (s, 3H) ppm.

The following intermediate examples have been prepared in the case of the imidazopyrimidines by reacting the corresponding aminoimidazoles with the properly substituted diethyl phenylmalonates followed by reaction with phosphorus oxychloride and if desired reduction of the dichloroderivate to the monochloroderivate in analogy to the procedures described above and in WO2009/021992.

In the case of the triazolopyrimidines the intermediates have been prepared by reacting the substituted aminotriazoles with the properly substituted diethyl phenylmalonates followed by reaction with phosphorus oxychloride and if desired reduction of the dichloroderivate to the monochloroderivate in analogy to the procedures described above and in WO2009/021992.

| | | | UPLC-MS |
|---|---|---|---|
| Intermediate | | | (rt in min) |
| Example | Structure/Name | 1H-NMR | resp. MS |
| Int-1-2 | 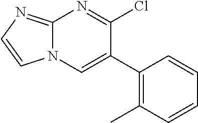 [SMILES: Cc1ccccc1-c1cn2ccnc2nc1Cl] | (300 MHz, CD₃OD): δ 8.82 (s, 1H), 7.84 (d, 1H), 7.72 (d, 1H), 7.19-7.44 (m, 4H), 2.21 (s, 3H). | |
| | 7-Chloro-6-o-tolyl-imidazo | | |
| | [1,2-a]pyrimidine | | |
| Int-1-3 | 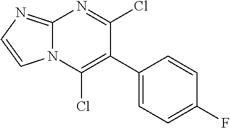 [SMILES: Fc1ccc(-c2c(Cl)nc3nccn3c2Cl)cc1] | (400 MHz, d6-DMSO): δ 8.22 (d, 1H), 8.07 (d, 1H), 7.45- 7.52 (m, 2H), 7.35-7.45 (m, 2H). | (CI, M + 1): 282 |
| | 5,7-Dichloro-6-(4-fluorophenyl)- | | |
| | imidazo[1,2-a]pyrimidine | | |
| Int-1-4 | 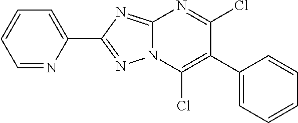 [SMILES: Clc1nc2nc(-c3ccccn3)nn2c(Cl)c1-c1ccccc1] | (300 MHz, d6-DMSO): δ 8.79 (d, 1H), 8.35 (d, 1H), 8.09 (dd, 1H), 7.41-7.69 (m, 6H). | (CI, M + 1): 342 |
| | 5,7-Dichloro-6-phenyl-2-pyridine-2- | | |
| | yl-[1,2,4]triazolo[1,5-a]pyrimidine | | |
| Int-1-6 | 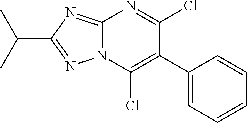 [SMILES: CC(C)c1nc2nc(Cl)c(-c3ccccc3)c(Cl)n2n1] | (300 MHz, d6-DMSO): δ 7.48-7.59 (m, 3H), 7.39- 7.48 (m, 2H); 3.12-3.30 (m, 1H, partly obscured by the water signal of the solvent), 1.35 (d, 6H). | RT = 1.33 min; m/z = 307 (ES+, M + 1) |
| | 5,7-Dichloro-2-isopropyl-6-phenyl- | | |
| | [1,2,4]triazolo[1,5-a]pyrimidine | | |
| Int-1-7 | 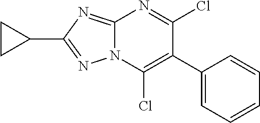 [SMILES: Clc1nc2nc(C3CC3)nn2c(Cl)c1-c1ccccc1] | (300 MHz, d6-DMSO): δ 7.45-7.60 (m, 3H), 7.38- 7.45 (m, 2H); 2.18-2.29 (m, 1H), 1.04-1.19 (m, 2H), 0.99- 1.04 (m, 2H). | RT = 1.35 min; m/z = 305 (ES+, M + 1) |
| | 5,7-Dichloro-2-cyclopropyl-6-phenyl- | | |
| | [1,2,4]triazolo[1,5-a]pyrimidine | | |
| Int-1-8 | 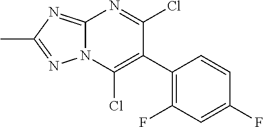 [SMILES: Cc1nc2nc(Cl)c(-c3ccc(F)cc3F)c(Cl)n2n1] | (300 MHz, d6-DMSO): δ 7.48-7.68 (m, 2H), 7.25-7.42 (m, 1H), 2.57 (s, 3H). | (CI, M + 1): 315 |
| | 5,7-Dichloro-6-(2,4-difluorophenyl)-2- | | |
| | methyl-[1,2,4]triazolo | | |
| | [1,5-a]pyrimidine | | |
| Int-1-9 | 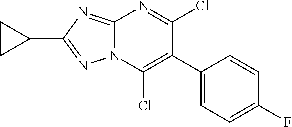 [SMILES: Fc1ccc(-c2c(Cl)nc3nc(C4CC4)nn3c2Cl)cc1] | (300 MHz, d6-DMSO): δ 7.32-7.58 (m, 4H), 2.18- 2.30 (m, 1H), 0.95-1.20 (m, 4H). | (CI, M + 1): 323 |
| | 5,7-Dichloro-2-cyclopropyl-6-(4- | | |
| | fluorophenyl)-[1,2,4]triazolo | | |
| | [1,5-a]pyrimidine | | |

### Intermediate Example Int-2-0

### {1-[4-(2-methyl-6-phenyl-[1,2,4]triazolo[1,5-a]pyrimidin-5-yl)-phenyl]-cyclobutyl}-carbamic acid tert-butyl ester

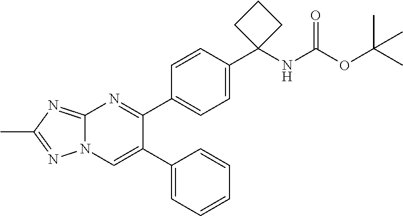 [SMILES: Cc1nc2nc(-c3ccc(C4(NC(=O)OC(C)(C)C)CCC4)cc3)c(-c3ccccc3)cn2n1]

A mixture of 5-chloro-2-methyl-6-phenyl[1,2,4]triazolo[1,5-a]pyrimidine (59 mg, 0.24 mmol), {1-[4-(4,4,5,5-tetramethyl-[1,3,2]dioxaborolan-2-yl)-phenyl]-cyclobutyl}-carbamic acid tert-butyl ester (134 mg), potassium carbonate (100 mg), Pd(PtBu₃)₂ (CAS No. 53199-31-8, 12.3 mg), NMP (0.11 mL), dioxane (0.46 mL) and water (0.2 mL) was heated under microwave irradiation (Biotage Initiator 60) at 80° C. for 10 min. The reaction was diluted with water, extracted with EtOAc and the organic layer dried and concentrated. Purification was achieved with chromatography on solica gel (Eluant: gradient elution DCM to 95% DCM/ethanol), followed by trituration with DIP/EtOAc to give the title compound (76 mg) which was used in the next step without further purification.

UPLC-MS: RT=1.33 min; m/z=456.28 (M+1).

The following intermediates were prepared by analogy from the appropriate 5-chloro[1,2,4]triazolo[1,5-a]pyrimidine and the appropriate boronic acid ester.

| Intermediate | | |
|---|---|---|
| Example | Structure/Name | Analytical Data |
| 2-1 | 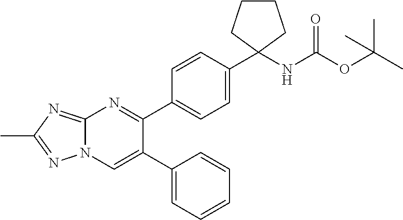 [SMILES: Cc1nc2nc(-c3ccc(C4(NC(=O)OC(C)(C)C)CCCC4)cc3)c(-c3ccccc3)cn2n1] | UPLC-MS: RT = 1.39 min; m/z = 470.31 (M + 1) |
| | {1-[4-(2-methyl-6-phenyl-[1,2,4]triazolo[1,5-a]pyrimidin-5- | |
| | yl)-phenyl]-cyclopentyl}-carbamic acid tert-butyl ester | |
| 2-2 | 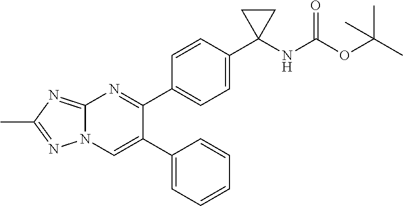 [SMILES: Cc1nc2nc(-c3ccc(C4(NC(=O)OC(C)(C)C)CC4)cc3)c(-c3ccccc3)cn2n1] | |
| | {1-[4-(2-methyl-6-phenyl-[1,2,4]triazolo[1,5-a]pyrimidin-5- | |
| | yl)-phenyl]-cyclopropyl}-carbamic acid tert-butyl ester | |
| 2-3 | 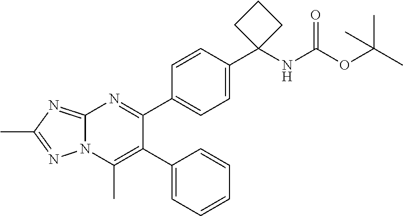 [SMILES: Cc1nc2nc(-c3ccc(C4(NC(=O)OC(C)(C)C)CCC4)cc3)c(-c3ccccc3)c(C)n2n1] | UPLC-MS: RT = 1.38 min; m/z = 470.33 (M + 1) |
| | {1-[4-(2,7-dimethyl-6-phenyl-[1,2,4]triazolo[1,5-a]pyrimidin- | |
| | 5-yl)-phenyl]-cyclobutyl}-carbamic acid tert-butyl ester | |
| 2-4 | 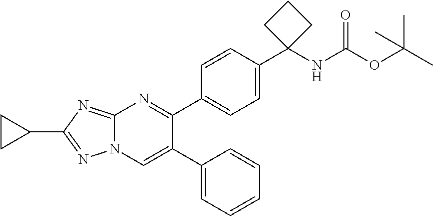 [SMILES: CC(C)(C)OC(=O)NC1(c2ccc(-c3nc4nc(C5CC5)nn4cc3-c3ccccc3)cc2)CCC1] | UPLC-MS: RT = 1.44 min; m/z = 482.29 (M + 1) |
| | {1-[4-(2-cyclopropyl-6-phenyl-[1,2,4]triazolo[1,5-a]pyrimidin- | |
| | 5-yl)-phenyl]-cyclobutyl}-carbamic acid tert-butyl ester | |

### Intermediate Example Int-3-0

### {1-[4-(6-Phenyl-imidazo[1,2-a]pyrimidin-7-yl)-phenyl]-cyclobutyl}-carbamic acid tert.-butylester

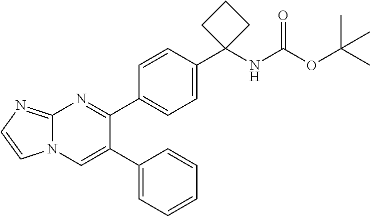 [SMILES: CC(C)(C)OC(=O)NC1(c2ccc(-c3nc4nccn4cc3-c3ccccc3)cc2)CCC1]

500 mg (2 mmol) 7-Chloro-6-phenyl-imidazo[1,2-b]pyrimidine (intermediate example Int-1-0) which contains 8% of 6-phenyl-imidazo[1,2-b]pyrimidine, 1.1 g (3 mmol) {1-[4-(4,4,5,5-tetramethyl-[1,3,2]dioxaborolan-2-yl)-phenyl]-cyclobutyl}-carbamic acid tert.-butyl ester, 73.6 mg (0.9 mmol) 1,1bis(diphenylphosphino)ferrocenedichloropalladium(II) and 636.8 mg (6 mmol) sodium carbonate (10%) are given in a microwave vial. 14 mL dioxane and 3.9 mL water which have been degassed are added. The reaction mixture is purged with nitrogen and stirred for 18 h at 105° C. The reaction has been carried out trice under the same conditions. The four reaction mixtures have been jointly worked up. They are poured on 150 mL water and 500 mL EtOAc and vigorously stirred for two hours. After separation of the organic phase the aqueous phase is extracted with ethyl acetate. The combined organic extracts are washed with water and dried over sodium sulfate. After removal of the drying agent the solvent is evaporated. However, in this residue only traces of the title compound have been found. Therefore, the aqueous phase has been diluted with 600 mL dichloromethane and extracted (no separation of the phases). After the weekend a solid has precipitated in the organic phase. The organic phase with the solid has been separated from the aqueous phase and the solvent is removed without drying the phase before. The residue is suspended in a small amount of dichloromethane, sucked off via a paper filter and washed with dichloromethane yielding 700 mg of the desired compound.

¹H-NMR (300 MHz, d6-DMSO): δ 9.01 (s, 1H), 7.92 (d, 1H), 7.78 (d, 1H), 7.50-7.62 (m, 1H), 7.20-7.40 (m, 8H), 2.25-2.42 (m, 4H), 1.88-2.05 (m, 1H), 1.65-1.82 (m, 1H), 0.98-1.42 (m, 9H).

### Intermediate Example Int-3-1

### {1-[4-(2-methyl-6-phenyl-imidazo[1,2-a]pyrimidin-7-yl)-phenyl]-cyclobutyl}-carbamic acid tert.-butyl ester

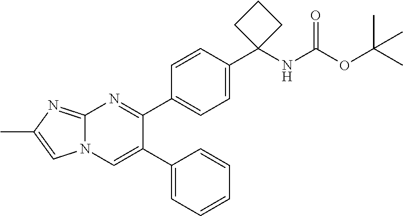 [SMILES: Cc1cn2cc(-c3ccccc3)c(-c3ccc(C4(NC(=O)OC(C)(C)C)CCC4)cc3)nc2n1]

150 mg (0.62 mmol) 7-Chloro-2-methyl-6-phenyl-imidazo[1,2-a]pyrimidine (intermediate example Int-1-1), 299 mg (0.8 mmol) {1-[4-(4,4,5,5-tetramethyl-[1,3,2]dioxaborolan-2-yl)-phenyl]-cyclobutyl}-carbamic acid tert.-butyl ester, 255 mg (1.85 mmol) potassium carbonate, 31.5 mg (0.06 mmol) bis(tri-tert.-butylphosphin)palladium(0) are given in 1.2 mL dioxane, 1.1 mL water and 0.3 mL NMP (no complete dissolution). The reaction mixture is stirred for 30′ in the microwave. The reaction mixture is poured on water and extracted twice with EtOAc. The combined organic extracts are washed with water and dried over sodium sulfate. After removal of the drying agent the solvent is evaporated and the residue is purified by chromatography on silicagel (eluents: dichloromethane/methanol) yielding 191.5 mg of the title compound which is contaminated.

MS (Cl+, M+1): 495

¹H-NMR (300 MHz, d6-DMSO): δ 8.90 (s, 1H), 7.63 (s, 1H), 7.10-7.36 (m, 9H), 2.19-2.42 (m, with a singulet within, 7H), 1.80-2.01 (m, 1H), 1.62-1.80 (m, 1H), 0.93-1.39 (m, 9H).

### Intermediate Example Int-3-2

### {1-[4-(5-cyclopropylamino-6-phenyl-imidazo[1,2-a]pyrimidin-7-yl]phenyl]-cyclobutyl}-carbamic acid tert.-butylester

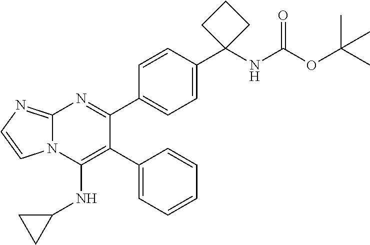 [SMILES: CC(C)(C)OC(=O)NC1(c2ccc(-c3nc4nccn4c(NC4CC4)c3-c3ccccc3)cc2)CCC1]

### Step 1: (7-chloro-6-phenyl-imidazo[1,2-a]pyrimidin-5-yl)-cyclopropylamine

648.5 mg (11.4 mmol) Cyclopropylamine are added to 1.2 g (4.5 mmol) 5,7-dichloro-6-phenylimidazo[1,2-a]pyrimidine, described in the intermediate example Int-1-0, in 12.6 mL DMF (no complete dissolution). The reaction mixture is heated for 20′ at 100° C. in the microwave. The light brown clear solution is poured on 50 mL water. After addition of 200 mL methyl-tert.-butylether and 10 mL dichloromethane the mixture is stirred for 30′. A precipitate has formed which is between the aqueous and the organic phase. The mixture is extracted twice with methyl-tert.-butylether. The combined organic extracts are worked up as usual, the solvent is evaporated and the residue, after spectral examination, is finally discarded. The precipitate which is now in the aqueous phase is sucked off, washed with water and dried yielding 694.4 mg (51%) of the title compound.

MS (ES+, M+1): 285

¹H-NMR (400 MHz, d6-DMSO): δ 8.05 (d, 1H), 7.65 (s, 1H), 7.55 (d, 1H), 7.31-7.45 (m, 5H), 1.81-1.90 (m, 1H), 0.42-0.51 (m, 2H), 0.09-0.17 (m, 2H).

### Step 2: {1-[4-(5-cyclopropylamino-6-phenyl-imidazo[1,2-a]pyrimidin-7-yl]phenyl]-cyclobutyl}-carbamic acid tert.-butylester

To 690 mg (2.4 mmol) (7-Chloro-6-phenyl-imidazo[1,2-a]pyrimidin-5-yl)-cyclopropylamine in 8.3 mL DME (no complete dissolution) are added 1.18 g (3.15 mmol) {1-[4-(4,4,5,5-tetramethyl-[1,3,2]dioxaborolan-2-yl)-phenyl]-cyclobutyl}-carbamic acid tert.-butyl ester, 5 mL aqueous sodium carbonate solution (10%) and 98.9 mg (0.121 mmol) 1,1bis(diphenylphosphino)ferrocenedichloropalladium(II). The reaction mixture is purged with argon and heated for 50′ at 90° C. in the microwave (no complete dissolution). After addition of 50 mL water and 150 mL dichloromethane the reaction mixture is vigorously stirred for one hour. The organic phase is separated and the aqueous phase is extracted twice with dichloromethane (150 mL each). The combined organic extracts are washed with water, dried, filtrated and the solvent is removed. The residue is purified by chromatography on silicagel (eluents: dichloromethane/methanol) yielding 407.6 mg of a product which contains the desired title compound only in minor amounts and additional fractions 242.6 mg which contains the desired compound strongly contaminated.

MS (ES+, M+1): 496

The following intermediate examples have been prepared in analogy as described for intermediate example Int-3-2.

| | | | UPLC-MS |
|---|---|---|---|
| Intermediate | | | (rt in min.) |
| Example | Structure/Name | 1H-NMR | resp. MS |
| Int-3-3 | 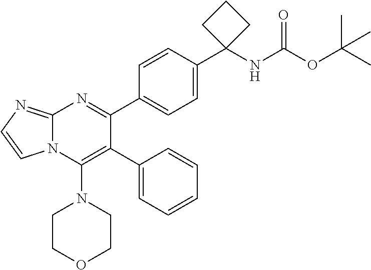 [SMILES: CC(C)(C)OC(=O)NC1(c2ccc(-c3nc4nccn4c(N4CCOCC4)c3-c3ccccc3)cc2)CCC1] | (300 MHz, d6-DMSO): δ 7.70-7.78 (m, 2H), 7.25- 7.38 (m, 3H), 7.03-7.25 (m, 6H), 3.49-3.61 (m, 4H), 2.58-2.95 (m, very br., 4H), 2.15-2.39 (m, 4H), 1.80-2.02 (m, 1H), 1.60-1.80 (m, 1H), 0.95-1.40 (m, 9H). | (ES+, M + 1): 526 |
| | {1-[4-(5-Morpholino-4-yl-6-phenyl- | | |
| | imidazo[1,2-a]pyrimidin-7-yl)-phenyl]- | | |
| | cyclobutyl}-carbamic acid tert.butyl | | |
| | ester | | |
| Int-3-4 | 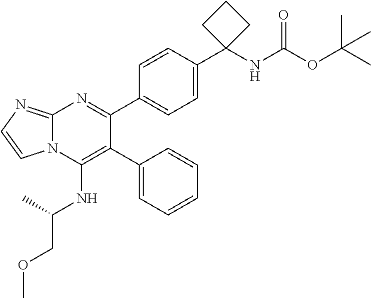 [SMILES: COC[C@H](C)Nc1c(-c2ccccc2)c(-c2ccc(C3(NC(=O)OC(C)(C)C)CCC3)cc2)nc2nccn12] | | |
| | (1-{4-[5-((S)-2-Methoxy-1-methyl- | | |
| | ethylamino)-6-phenyl-imidazo[1,2- | | |
| | a]pyrimidin-7-yl]-phenyl}-cyclobutyl)- | | |
| | carbamic acid tert.butyl ester | | |
| Int-3-5 | 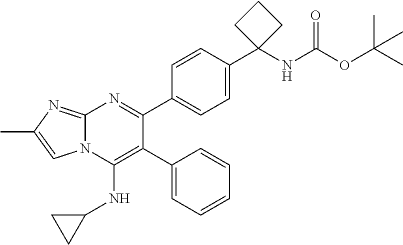 [SMILES: Cc1cn2c(NC3CC3)c(-c3ccccc3)c(-c3ccc(C4(NC(=O)OC(C)(C)C)CCC4)cc3)nc2n1] | | RT = 1.35 min; m/z = 510 (ES+, M + 1) |
| | {1-[4-(5-cyclopropylamino-2-methyl-6- | | |
| | phenyl-imidazo[1,2-a]pyrimidin-7- | | |
| | yl]phenyl]-cyclobutyl}-carbamic acid | | |
| | tert.-butylester | | |
| Int-3-6 | 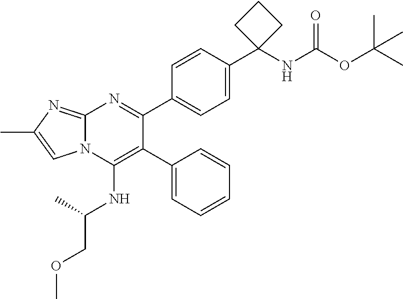 [SMILES: COC[C@H](C)Nc1c(-c2ccccc2)c(-c2ccc(C3(NC(=O)OC(C)(C)C)CCC3)cc2)nc2nc(C)cn12] | | |
| | (1-{4-[5-((S)-2-Methoxy-1-methyl- | | |
| | ethylamino)-2-methyl-6-phenyl- | | |
| | imidazo[1,2-a]pyrimidin-7-yl]-phenyl}- | | |
| | cyclobutyl)-carbamic acid tert.butyl | | |
| | ester | | |
| Int-3-7 | 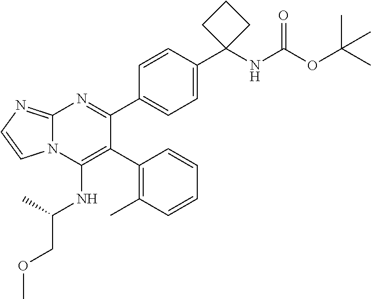 [SMILES: COC[C@H](C)Nc1c(-c2ccccc2C)c(-c2ccc(C3(NC(=O)OC(C)(C)C)CCC3)cc2)nc2nccn12] | | RT = 1.11 min; m/z = 542 (ES+, M + 1) Method B |
| | (1-{4-[5-((S)-2-Methoxy-1-methyl- | | |
| | ethylamino)-6-o-tolyl-imidazo[1,2- | | |
| | a]pyrimidin-7-yl]-phenyl}-cyclobutyl)- | | |
| | carbamic acid tert.butyl ester | | |
| Int-3-8 | 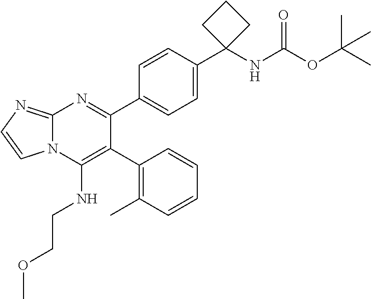 [SMILES: COCCNc1c(-c2ccccc2C)c(-c2ccc(C3(NC(=O)OC(C)(C)C)CCC3)cc2)nc2nccn12] | | RT = 1.07 min; m/z = 526 (ES−, M − 1) Method B |
| | (1-{4-[5-(2-Methoxy-1-methyl- | | |
| | ethylamino)-6-o-tolyl-imidazo[1,2- | | |
| | a]pyrimidin-7-yl]-phenyl}-cyclobutyl)- | | |
| | carbamic acid tert.butyl ester | | |

### Intermediate Example Int-4-0

### {1-[4-(7-tert.-butylamino-6-phenyl-2-pyridine-2-yl-[1,2,4]triazolo[1,5-a]pyrimidin-5-yl)-phenyl]-cyclobutyl}-carbamic acid tert.-butyl ester

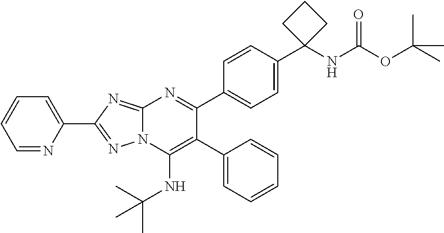 [SMILES: CC(C)(C)Nc1c(-c2ccccc2)c(-c2ccc(C3(NC(=O)OC(C)(C)C)CCC3)cc2)nc2nc(-c3ccccn3)nn12]

### Step 1: tert.-butyl-(5-chloro-6-phenyl-2-pyridine-2-yl-[1,2,4]triazolo[1,5-a]pyrimidin-7-yl)-amine

1 g (2.9 mmol) 5,7-Dichloro-6-phenyl-2-pyridine-2-yl-[1,2,4]triazolo[1,5-a]pyrimidine (intermediate example Int-1-4) and 0.53 g (7.3 mmol) tert.-butylamine are dissolved in 8.1 mL DMF (no complete dissolution). The reaction mixture is heated in the microwave for 20′ at 100° C. The solvent is evaporated and the residue is purified by chromatography on silicagel (eluents: dichloromethane/methanol) yielding 640 mg (57.8%) of the title compound.

¹H-NMR (300 MHz, d6-DMSO): δ 8.76 (d, 1H), 8.23 (d, 1H), 8.01 (dd, 1H), 7.40-7.64 (m, 6H), 1.39 (s, 9H).

### Step 2: {1-[4-(7-tert.-butylamino-6-phenyl-2-pyridine-2-yl-[1,2,4]triazolo[1,5-a]pyrimidin-5-yl)-phenyl]-cyclobutyl}-carbamic acid tert.-butyl ester

200 mg (0.53 mmol) tert.-Butyl-(5-chloro-6-phenyl-2-pyridine-2-yl-[1,2,4]triazolo[1,5-a]pyrimidin-7-yl)-amine are given in 1.8 mL DME (no complete dissolution). After addition of 394 mg (1.05 mmol) {1-[4-(4,4,5,5-tetramethyl-[1,3,2]dioxaborolan-2-yl)-phenyl]-cyclobutyl}-carbamic acid tert.-butyl ester, 0.97 ml aqueous sodium carbonate (10%), and 19.3 mg (23.8 mmol) 1,1bis(diphenylphosphino)ferrocenedichloropalladium(II) the reaction mixture is evaporated three times and purged with argon and heated for 18 hours at 90° C. The reaction mixture is diluted with water (15 mL) and dichloromethane (30 mL) and stirred for one hour at room temperature. The organic phase is separated and the aqueous phase is extracted twice with dichloromethane (30 ml each). The combined organic extracts are washed with water and brine, dried and filtrated. After removal of the solvent the residue is purified by chromatography on silicagel (eluents: dichloromethane/methanol) yielding 310 mg of the title compound which is however contaminated.

MS (ES+, M+1): 590

¹H-NMR (400 MHz, d6-DMSO): δ 8.79 (d, 1H), 8.29 (d, 1H), 8.01 (dd, 1H), 7.12-7.64 (m, 10H), 2.19-2.42 (m, 4H), 1.82-2.02 (m, 1H), 1.60-1.82 (m, 1H), 1.18-1.49 (m, 18H).

The following intermediate examples have been prepared in analogy as described for intermediate example Int-4-0.

| | | | UPLC-MS |
|---|---|---|---|
| Intermediate | | | (rt in min.) |
| Example | Structure/Name | 1H-NMR | resp. MS |
| Int-4.1 | 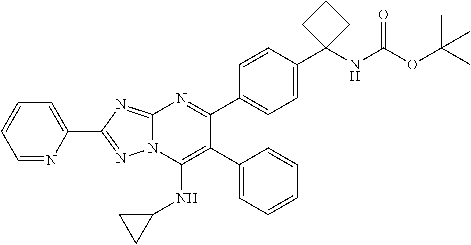 [SMILES: CC(C)(C)OC(=O)NC1(c2ccc(-c3nc4nc(-c5ccccn5)nn4c(NC4CC4)c3-c3ccccc3)cc2)CCC1] | | RT = 1.42 min; m/z = 572 (ES−, M − 1) |
| | {1-[4-(7-Cyclopropylamino-6-phenyl- | | |
| | 2-pyridine-2-yl-[1,2,4]triazolo[1,5- | | |
| | a]pyrimidin-5-yl)-phenyl]-cyclobutyl}- | | |
| | carbamic acid tert.-butyl ester | | |
| Int-4-2 | 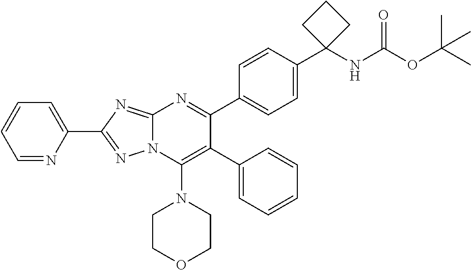 [SMILES: CC(C)(C)OC(=O)NC1(c2ccc(-c3nc4nc(-c5ccccn5)nn4c(N4CCOCC4)c3-c3ccccc3)cc2)CCC1] | | RT = 1.38 min; m/z = 604 (ES+, M + 1) |
| | {1-[4-(7-Morpholine-4-yl-6-phenyl-2- | | |
| | pyridine-2-yl-[1,2,4]triazolo[1,5- | | |
| | a]pyrimidin-5-yl)-phenyl]-cyclobutyl}- | | |
| | carbamic acid tert-butyl ester | | |
| Int-4-3 | 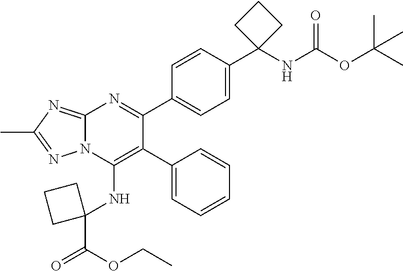 [SMILES: CCOC(=O)C1(Nc2c(-c3ccccc3)c(-c3ccc(C4(NC(=O)OC(C)(C)C)CCC4)cc3)nc3nc(C)nn23)CCC1] | | RT = 1.49 min; m/z = 595 (ES−, M − 1) |
| | 1-{5-[4-(1-tert-Butoxycarbonylamino- | | |
| | cyclobutyl)-phenyl]-2-methyl-6- | | |
| | phenyl-[1,2,4]triazolo[1,5-a]pyrimidin- | | |
| | 7-ylamino}-cyclobutanecarboxylic acid | | |
| | ethyl ester | | |
| Int-4-4 | 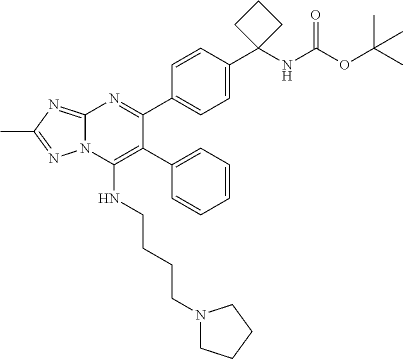 [SMILES: Cc1nc2nc(-c3ccc(C4(NC(=O)OC(C)(C)C)CCC4)cc3)c(-c3ccccc3)c(NCCCCN3CCCC3)n2n1] | | |
| | (1-{4-[2-Methyl-6-phenyl-7-(4- | | |
| | pyrrolidine-1-yl-butylamino)- | | |
| | [1,2,4]triazolo[1,5-a]pyrimidin-5-yl]- | | |
| | phenyl}-cyclobutyl)-carbamic acid tert- | | |
| | butyl ester | | |
| Int-4-5 | 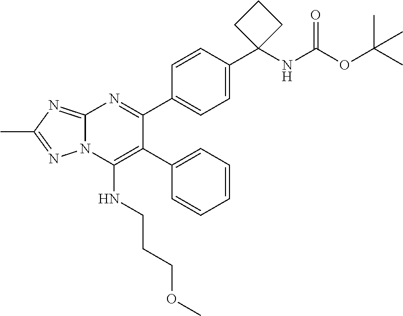 [SMILES: COCCCNc1c(-c2ccccc2)c(-c2ccc(C3(NC(=O)OC(C)(C)C)CCC3)cc2)nc2nc(C)nn12] | | |
| | (1-{4-[7-(3-Methoxy-propylamino)-2- | | |
| | methyl-6-phenyl-[1,2,4]triazolo[1,5- | | |
| | a]pyrimidin-5-yl]-phenyl}-cyclobutyl)- | | |
| | carbamic acid tert-butyl ester | | |
| Int-4-6 | 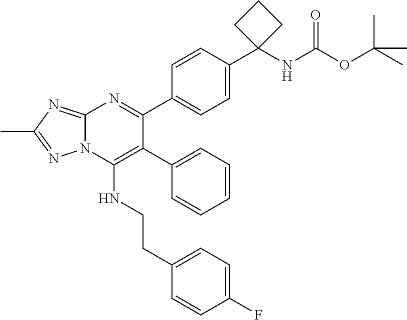 [SMILES: Cc1nc2nc(-c3ccc(C4(NC(=O)OC(C)(C)C)CCC4)cc3)c(-c3ccccc3)c(NCCc3ccc(F)cc3)n2n1] | | |
| | [1-(4-{7-[2-(4-Fluorophenyl)- | | |
| | ethylamino]-2-methyl-6-phenyl- | | |
| | [1,2,4]triazolo[1,5-a]pyrimidin-5-yl}- | | |
| | phenyl)-cyclobutyl)-carbamic acid tert- | | |
| | butyl ester | | |
| Int-4-7 | 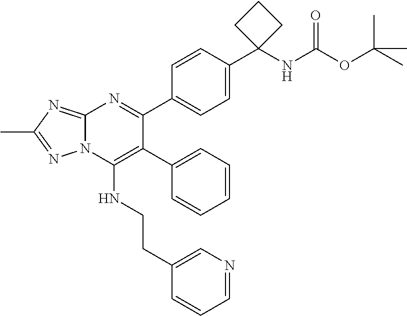 [SMILES: Cc1nc2nc(-c3ccc(C4(NC(=O)OC(C)(C)C)CCC4)cc3)c(-c3ccccc3)c(NCCc3cccnc3)n2n1] | | |
| | (1-{4-[2-Methyl-6-phenyl-7-(2- | | |
| | pyridine-3-yl-ethylamino) | | |
| | [1,2,4]triazolo[1,5-a]pyrimidin-5-yl]- | | |
| | phenyl}-cyclobutyl)-carbamic acid tert- | | |
| | butyl ester | | |
| Int-4-8 | 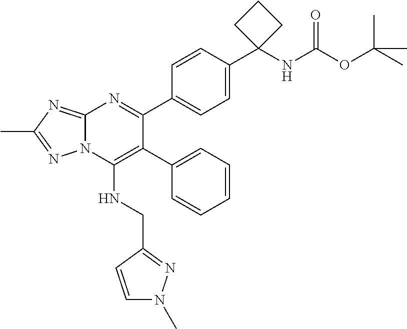 [SMILES: Cc1nc2nc(-c3ccc(C4(NC(=O)OC(C)(C)C)CCC4)cc3)c(-c3ccccc3)c(NCc3ccn(C)n3)n2n1] | | |
| | [1-(4-{2-Methyl-7-[(1-methyl-1H- | | |
| | pyrazol-3-ylmethyl)-amino]-6-phenyl- | | |
| | [1,2,4]triazolo[1,5-a]pyrimidin-5-yl]- | | |
| | phenyl}-cyclobutyl)-carbamic acid tert- | | |
| | butyl ester | | |

### Intermediate Example Int-5-0

### {1-[4-(2-bromo-6-phenyl-[1,2,4]triazolo[1,5-a]pyrimidin-5-yl)-phenyl]-cyclobutyl}-carbamic acid tert-butyl ester

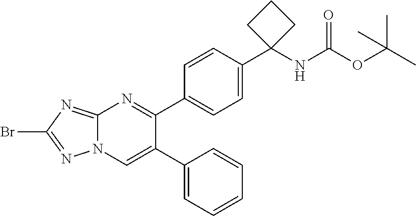 [SMILES: CC(C)(C)OC(=O)NC1(c2ccc(-c3nc4nc(Br)nn4cc3-c3ccccc3)cc2)CCC1]

### Step 1: 2-bromo-5-chloro-6-phenyl[1,2,4]triazolo[1,5-a]pyrimidine

The title compound is prepared in analogy to 5-chloro-2-methyl-6-phenyl[1,2,4]triazolo[1,5-a]pyrimidine above by using 5-bromo-2H-[1,2,4]triazol-3-ylamine (prepared according to WO2003/80614) in the first step.

¹H NMR (300 MHz, d6-DMSO): δ 9.60 (s, 1H), 7.54-7.56 (m, 5H) ppm.

### Step 2: {1-[4-(2-bromo-6-phenyl-[1,2,4]triazolo[1,5-a]pyrimidin-5-yl)-phenyl]-cyclobutyl}-carbamic acid tert-butyl ester

To a mixture of 2-bromo-5-chloro-6-phenyl[1,2,4]triazolo[1,5-a]pyrimidine (870 mg) and {1-[4-(4,4,5,5-tetramethyl-[1,3,2]dioxaborolan-2-yl)-phenyl]-cyclobutyl}-carbamic acid tert-butyl ester (1.15 g) in DME (9.5 mL), was added 10% aqueous sodium carbonate (6.2 mL) and Pd(dppf)Cl₂ (114 mg). The resulting orange red suspension was heated under microwave irradiation at 100° C. for 50 minutes. On cooling the mixture was partitioned between DCM and water, the aqueous phase extracted with DCM and the combined organic phases washed with brine, dried and concentrated. Purification was achieved by chromatography on silica gel to give slightly impure title compound (360 mg) as a white foam.

UPLC-MS: RT=1.46 min; m/z=471.28 (M+1).

### Intermediate Example Int-6-0

### (1-{4-[2-(2-methoxy-ethylamino)-6-phenyl-[1,2,4]triazolo[1,5-a]pyrimidin-5-yl]-phenyl}-cyclobutyl)-carbamic acid tert-butyl ester

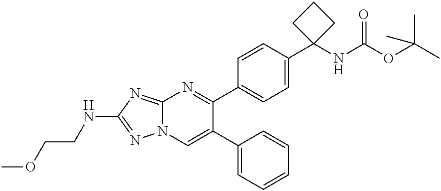 [SMILES: COCCNc1nc2nc(-c3ccc(C4(NC(=O)OC(C)(C)C)CCC4)cc3)c(-c3ccccc3)cn2n1]

A solution of {1-[4-(2-bromo-6-phenyl-[1,2,4]triazolo[1,5-a]pyrimidin-5-yl)-phenyl]-cyclobutyl}-carbamic acid tert-butyl ester (180 mg) and 2-methoxyethylamine (52 mg, 0.06 mL) in NMP (2 mL) was heated under microwave irradiation at 100° C. for 30 min. UPLC-MS indicated incomplete conversion. Further 2-methoxyethylamine (52 mg, 0.06 mL) was added and the mixture was heated overnight at 100° C. whereupon UPLC-MS indicated starting material was consumed. The volatiles were removed in vacuo and the crude title compound was used directly in the next step without further purification.

UPLC-MS: RT=1.34 min; m/z=515.70 (M+1).

The following intermediates were prepared in analogy by reacting {1-[4-(2-bromo-6-phenyl-[1,2,4]triazolo[1,5-a]pyrimidin-5-yl)phenyl]-cyclobutyl}-carbamic acid tert-butyl ester with the appropriate amine.

| Intermediate | | |
|---|---|---|
| Example | Structure/Name | Analytical Data |
| Int-6-0 | 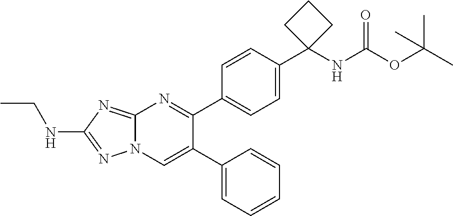 [SMILES: CCNc1nc2nc(-c3ccc(C4(NC(=O)OC(C)(C)C)CCC4)cc3)c(-c3ccccc3)cn2n1] | UPLC-MS: RT = 1.38 min; m/z = 485.70 (M + 1) |
| | {1-[4-(2-ethylamino-6-phenyl- | |
| | [1,2,4]triazolo[1,5-a]pyrimidin-5-yl)-phenyl]- | |
| | cyclobutyl}-carbamic acid tert-butyl ester | |

### Intermediate Example Int-7-0

### (1-{4-[2-(1-methyl-1H-pyrazol-4-yl)-6-phenyl-[1,2,4]triazolo[1,5-a]pyrimidin-5-yl]-phenyl}-cyclobutyl)-carbamic acid tert-butyl ester

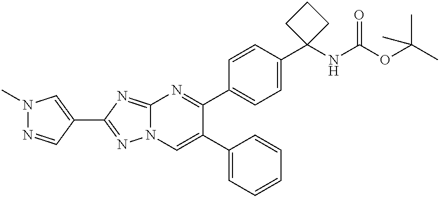 [SMILES: Cn1cc(-c2nc3nc(-c4ccc(C5(NC(=O)OC(C)(C)C)CCC5)cc4)c(-c4ccccc4)cn3n2)cn1]

A mixture of {1-[4-(2-bromo-6-phenyl-[1,2,4]triazolo[1,5-a]pyrimidin-5-yl)-phenyl]-cyclobutyl}-carbamic acid tert-butyl ester (180 mg), 1-methyl-1H-pyrazole-4-boronic acid pinacol ester (79 mg), Pd(dppf)Cl₂ (14 mg), 10% aqueous sodium carbonate (0.75 mL) and DME (1.175 mL) was heated under microwave irradiation at 100° C. for 1 hour. On cooling the mixture was diluted with water, extracted with DCM and the combined organic phase washed with brine, dried and concentrated. Purification by chromatography gave the title compound.

UPLC-MS: RT=1.45 min; m/z=522.50 (M+1).

### Intermediate Example Int-8-0

### {1-[4-(2-methylamino-6-phenyl-[1,2,4]triazolo[1,5-a]pyrimidin-5-yl)-phenyl]-cyclobutyl}-carbamic acid tert-butyl ester

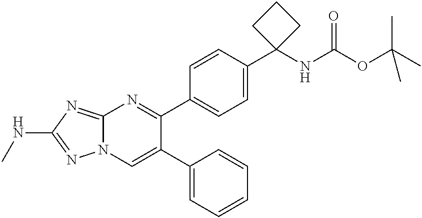 [SMILES: CNc1nc2nc(-c3ccc(C4(NC(=O)OC(C)(C)C)CCC4)cc3)c(-c3ccccc3)cn2n1]

{1-[4-(2-methylamino-6-phenyl-[1,2,4]triazolo[1,5-a]pyrimidin-5-yl)-phenyl]-cyclobutyl}-carbamic acid tert-butyl ester was prepared in analogy to {1-[4-(2-methyl-6-phenyl-[1,2,4]triazolo[1,5-a]pyrimidin-5-yl)phenyl]-cyclobutyl}-carbamic acid tert-butyl ester Steps 1-4, except that in Step 1 3-amino-5-methyltriazole was replaced with N*3*-methyl-1H-[1,2,4]triazole-3,5-diamine (prepared using the procedure below).

UPLC-MS: RT=1.30 min; m/z=471.28 (M+1);

¹H NMR (400 MHz, d6-DMSO): δ 9.04 (s, 1H), 7.58 (br s), 7.22-7.32 (m, 9H), 6.92 (q, 1H), 2.86 (d, 3H), 2.32 (br m, 4H), 1.96 (br m, 1H), 1.75 (br m, 1H), 1.32 (br s, 6H), 1.10 (br s, 3H) ppm.

### Preparation of N*3*-methyl-1H-[1,2,4]triazole-3,5-diamine

A suspension of N-cyano-N,S-dimethylisothiourea (14.22 g, 0.108 mol) and 80% hydrazine hydrate (13.1 mL) in ethanol (54 mL) was heated at reflux for 2 hours, whereupon the suspension dissolved. On cooling the mixture was concentrated in vacuo to a colourless oil which was triturated with petroleum ether at 0° C. until precipitation was observed. The solid was filtered, washed with petroleum ether and dried to give the title compound as a violet solid (12.75 g) which was used completely in the next step without further purification.

MS (ESI, M+1): 114

The following intermediates were prepared in analogy to {1-[4-(2-methylamino-6-phenyl-[1,2,4]triazolo[1,5-a]pyrimidin-5-yl)-phenyl]-cyclobutyl}-carbamic acid tert-butyl ester by using the appropriate diaminotriazole in Step 1.

| Intermediate | | |
|---|---|---|
| Example | Structure/Name | Analytical Data |
| Int-8-1 | 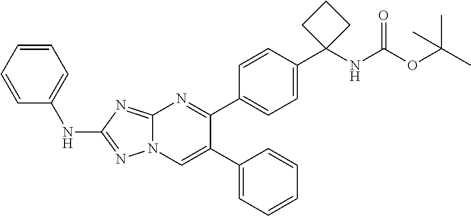 [SMILES: CC(C)(C)OC(=O)NC1(c2ccc(-c3nc4nc(Nc5ccccc5)nn4cc3-c3ccccc3)cc2)CCC1] | UPLC-MS: RT = 1.51 min; m/z = 533.38 (M + 1) |
| | {1-[4-(6-phenyl-2-phenylamino- | |
| | [1,2,4]triazolo[1,5-a]pyrimidin-5-yl)-phenyl]- | |
| | cyclobutyl}-carbamic acid tert-butyl ester | |
| Int-8-2 | 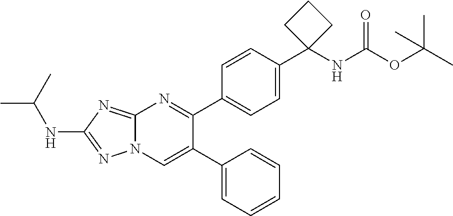 [SMILES: CC(C)Nc1nc2nc(-c3ccc(C4(NC(=O)OC(C)(C)C)CCC4)cc3)c(-c3ccccc3)cn2n1] | UPLC-MS: RT = 1.44 min; m/z = 499.1 (M + 1) |
| | {1-[4-(2-isopropylamino-6-phenyl- | |
| | [1,2,4]triazolo[1,5-a]pyrimidin-5-yl)-phenyl]- | |
| | cyclobutyl}-carbamic acid tert-butyl ester | |
| Int-8-3 | 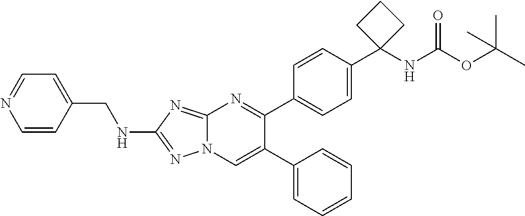 [SMILES: CC(C)(C)OC(=O)NC1(c2ccc(-c3nc4nc(NCc5ccncc5)nn4cc3-c3ccccc3)cc2)CCC1] | UPLC-MS: RT = 1.11 min; m/z = 548.71 (M + 1) |
| | [1-(4-{6-phenyl-2-[(pyridin-4-ylmethyl)- | |
| | amino]-[1,2,4]triazolo[1,5-a]pyrimidin-5-yl}- | |
| | phenyl)-cyclobutyl]-carbamic acid tert-butyl | |
| | ester | |

### Intermediate Example Int-9-0

### {1-[4-(7-isopropylamino-2-methyl-6-phenyl-[1,2,4]triazolo[1,5-a]pyrimidin-5-yl)-phenyl]-cyclobutyl}-carbamic acid tert-butyl ester

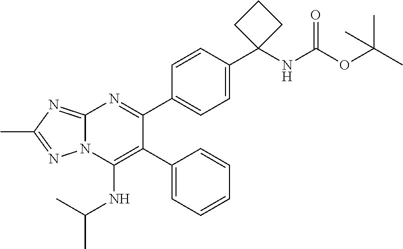 [SMILES: Cc1nc2nc(-c3ccc(C4(NC(=O)OC(C)(C)C)CCC4)cc3)c(-c3ccccc3)c(NC(C)C)n2n1]

### Step 1: (5-chloro-2-methyl-6-phenyl-[1,2,4]triazolo[1,5-a]pyrimidin-7-yl)-isopropyl-amine

The following reaction was performed in duplicate and combined in the work up. A suspension of 5,7-dichloro-2-methyl-6-phenyl-[1,2,4]triazolo[1,5-a]pyrimidine (2 g) in DMF (15.5 mL) was treated with isopropylamine (1.52 mL) and the mixture heated under microwave irradiation at 100° C. for 15 minutes. On cooling the combined reactions were diluted with water and the resulting suspension filtered and dried to give the title compound (2.8 g).

¹H NMR (400 MHz, d6-DMSO): δ 7.42-7.51 (m, 6H), 3.28 (m, 1H), 2.47 (s, 3H), 0.96 (d, 6H) ppm

### Step 2: {1-[4-(7-isopropylamino-2-methyl-6-phenyl-[1,2,4]triazolo[1,5-a]pyrimidin-5-yl)phenyl]-cyclobutyl}-carbamic acid tert-butyl ester

A mixture of (5-chloro-2-methyl-6-phenyl-[1,2,4]triazolo[1,5-a]pyrimidin-7-yl)-isopropyl-amine (374 mg) and {1-[4-(4,4,5,5-tetramethyl-[1,3,2]dioxaborolan-2-yl)-phenyl]-cyclobutyl}-carbamic acid tert-butyl ester (926 mg) in DME (6 mL) was treated with 10% aqueous sodium carbonate (2.49 mL) and placed under nitrogen. Pd(dppf)Cl₂ (45.6 mg) was added and the mixture heated under microwave irradiation at 100° C. for 45 minutes. On cooling the reaction was partitioned between water and EtOAc, the aqueous phase extracted with EtOAc and the combined organic phases dried and concentrated. Purification was achieved by chromatography on silica gel (eluent: gradient elution 100% hexane to 100% EtOAc) to afford the title compound, slightly contaminated with (5-chloro-2-methyl-6-phenyl-[1,2,4]triazolo[1,5-a]pyrimidin-7-yl)-isopropyl-amine.

UPLC-MS: RT=1.47 min; m/z=513.32 (M+1).

The following intermediates were prepared in analogy to {1-[4-(7-isopropylamino-2-methyl-6-phenyl-[1,2,4]triazolo[1,5-a]pyrimidin-5-yl)phenyl]-cyclobutyl}-carbamic acid tert-butyl ester by using the appropriate intermediate and amine in Step 1.

| Intermediate | | |
|---|---|---|
| Example | Structure/Name | Analytical Data |
| Int-9-1 | 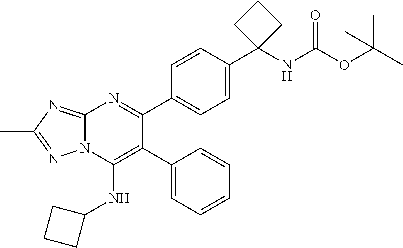 [SMILES: Cc1nc2nc(-c3ccc(C4(NC(=O)OC(C)(C)C)CCC4)cc3)c(-c3ccccc3)c(NC3CCC3)n2n1] | UPLC-MS: RT = 1.51 min; m/z = 525.32 (M + 1) |
| | {1-[4-(7-cyclobutylamino-2-methyl-6- | |
| | phenyl-[1,2,4]triazolo[1,5-a]pyrimidin-5- | |
| | yl)-phenyl]-cyclobutyl}-carbamic acid tert- | |
| | butyl ester | |
| Int-9-2 | 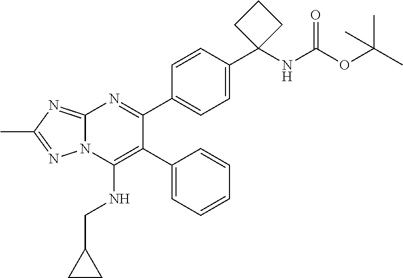 [SMILES: Cc1nc2nc(-c3ccc(C4(NC(=O)OC(C)(C)C)CCC4)cc3)c(-c3ccccc3)c(NCC3CC3)n2n1] | UPLC-MS: RT = 1.43 min; m/z = 525.35 (M + 1) |
| | (1-{4-[7-(cyclopropylmethyl-amino)-2- | |
| | methyl-6-phenyl-[1,2,4]triazolo[1,5- | |
| | a]pyrimidin-5-yl]-phenyl}-cyclobutyl)- | |
| | carbamic acid tert-butyl ester | |
| Int-9-3 | 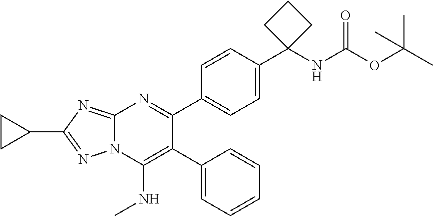 [SMILES: CNc1c(-c2ccccc2)c(-c2ccc(C3(NC(=O)OC(C)(C)C)CCC3)cc2)nc2nc(C3CC3)nn12] | UPLC-MS: RT = 1.38 min; m/z = 511.31 (M + 1) |
| | {1-[4-(2-cyclopropyl-7-methylamino-6- | |
| | phenyl-[1,2,4]triazolo[1,5-a]pyrimidin-5- | |
| | yl)-phenyl]-cyclobutyl}-carbamic acid tert- | |
| | butyl ester | |
| Int-9-4 | 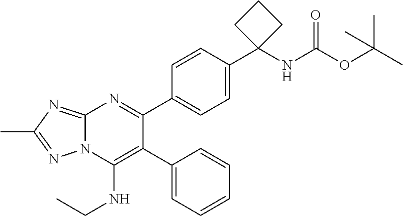 [SMILES: CCNc1c(-c2ccccc2)c(-c2ccc(C3(NC(=O)OC(C)(C)C)CCC3)cc2)nc2nc(C)nn12] | UPLC-MS: RT = 1.34 min; m/z = 499.36 (M + 1) |
| | {1-[4-(7-ethylamino-2-methyl-6-phenyl- | |
| | [1,2,4]triazolo[1,5-a]pyrimidin-5-yl)- | |
| | phenyl]-cyclobutyl}-carbamic acid tert- | |
| | butyl ester | |
| Int-9-5 | 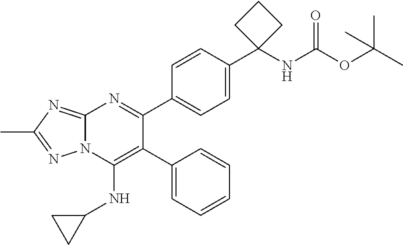 [SMILES: Cc1nc2nc(-c3ccc(C4(NC(=O)OC(C)(C)C)CCC4)cc3)c(-c3ccccc3)c(NC3CC3)n2n1] | UPLC-MS: RT = 1.43 min; m/z = 511.34 (M + 1) |
| | {1-[4-(7-cyclopropylamino-2-methyl-6- | |
| | phenyl-[1,2,4]triazolo[1,5-a]pyrimidin-5- | |
| | yl)-phenyl]-cyclobutyl}-carbamic acid tert- | |
| | butyl ester | |
| Int-9-6 | 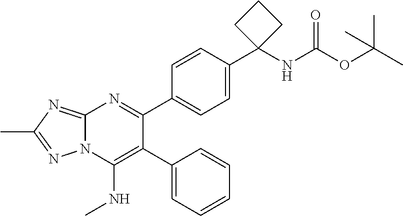 [SMILES: CNc1c(-c2ccccc2)c(-c2ccc(C3(NC(=O)OC(C)(C)C)CCC3)cc2)nc2nc(C)nn12] | UPLC-MS: RT = 1.30 min; m/z = 485.29 (M + 1) |
| | {1-[4-(2-methyl-7-methylamino-6-phenyl- | |
| | [1,2,4]triazolo[1,5-a]pyrimidin-5-yl)- | |
| | phenyl]-cyclobutyl}-carbamic acid tert- | |
| | butyl ester | |
| Int-9-7 | 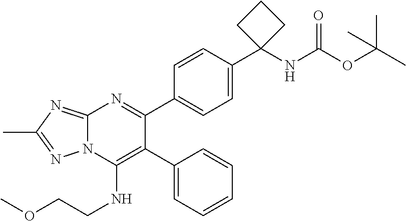 [SMILES: COCCNc1c(-c2ccccc2)c(-c2ccc(C3(NC(=O)OC(C)(C)C)CCC3)cc2)nc2nc(C)nn12] | UPLC-MS: RT = 1.36 min; m/z = 529.34 (M + 1) |
| | (1-{4-[7-(2-methoxy-ethylamino)-2- | |
| | methyl-6-phenyl-[1,2,4]triazolo[1,5- | |
| | a]pyrimidin-5-yl]-phenyl}-cyclobutyl)- | |
| | carbamic acid tert-butyl ester | |
| Int-9-8 | 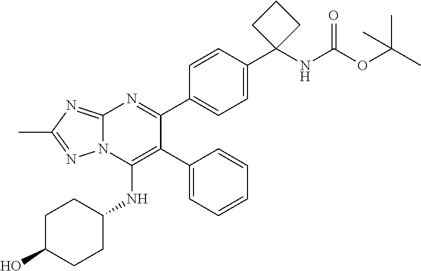 | UPLC-MS: RT = 1.32 min; m/z = 595.68 (M + 1) |
| | (1-{4-[7-(4-hydroxy-cyclohexylamino)-2- | |
| | methyl-6-phenyl-[1,2,4]triazolo[1,5- | |
| | a]pyrimidin-5-yl]-phenyl}-cyclobutyl)- | |
| | carbamic acid tert-butyl ester | |

### Intermediate Example Int-10-0

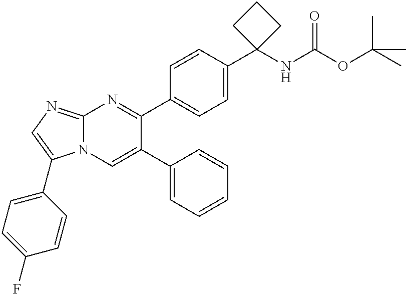 [SMILES: CC(C)(C)OC(=O)NC1(c2ccc(-c3nc4ncc(-c5ccc(F)cc5)n4cc3-c3ccccc3)cc2)CCC1]

### (1-{4-[3-(4-Fluorophenyl)-6-phenyl-imidazo[1,2-a]pyrimidin-7-yl]-phenyl}-cyclobutyl)-carbamic acid tert.-butyl ester

100 mg (0.19 mmol) {1-[4-(3-Bromo-6-phenyl-imidazo[1,2-a]pyrimidin-7-yl)-phenyl]-cyclobutyl}-carbamic acid tert.-butyl ester, 53.9 mg (0.39 mmol) 4-(fluorophenyl)-boronic acid, 15.7 mg (0.02 mmol) 1,1bis(diphenylphosphino)ferrocenedichloro-palladium(II) and 61.2 mg (0.58 mmol) sodium carbonate in 2.07 mL dioxane and 0.29 mL water (both solvents had been degassed) were heated in the microwave for 45′ at 105° C. The reaction mixture was poured on water/dichloromethane/saturated ammonium chloride and vigorously stirred for 30′. The organic phase was separated, washed twice with brine, dried (sodium sulfate) and filtrated. The solvent was evaporated and the crude residue (175 mg>100%) was used in the next step without further purification.

UPLC-MS: RT=1.44 min; m/z=535 (ES+, M+1)

The following intermediate examples had been prepared in analogy according to intermediate example Int-10-0 by reacting {1-[4-(3-bromo-6-phenyl-imidazo[1,2-a]pyrimidin-7-yl)-phenyl]-cyclobutyl}-carbamic acid tert.-butyl ester with the appropriate boronic acids.

| Intermediate | | | UPLC-MS resp. |
|---|---|---|---|
| example | Structure/Name | 1H-NMR | MS |
| Int-10-1 | 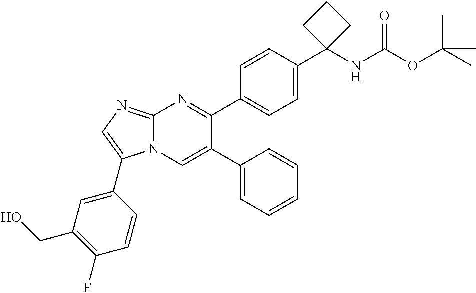 [SMILES: CC(C)(C)OC(=O)NC1(c2ccc(-c3nc4ncc(-c5ccc(F)c(CO)c5)n4cc3-c3ccccc3)cc2)CCC1] | none | RT = 1.29 min; m/z = 565 (ES+, M + 1) |
| | (1-{4-[3-(4-Fluoro-3-hydroxymethyl- | | |
| | phenyl)-6-phenyl-imidazo[1,2- | | |
| | a]pyrimidin-7-yl]-phenyl}-cyclobutyl)- | | |
| | carbamic acid tert.-butyl ester | | |
| Int-10-2 | 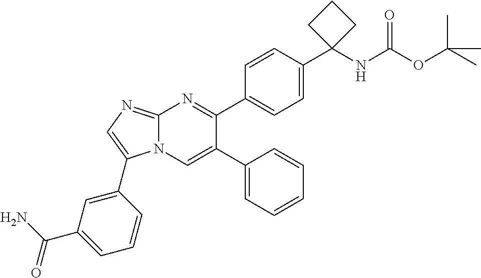 [SMILES: CC(C)(C)OC(=O)NC1(c2ccc(-c3nc4ncc(-c5cccc(C(N)=O)c5)n4cc3-c3ccccc3)cc2)CCC1] | none | RT = 1.20 min; m/z = 560 (ES+, M + 1) |
| | (1-{4-[3-(3-Carbamoyl-phenyl)-6-phenyl- | | |
| | imidazo[1,2-a]pyrimidin-7-yl]-phenyl}- | | |
| | cyclobutyl)-carbamic acid tert.-butyl | | |
| | ester | | |
| Int-10-3 | 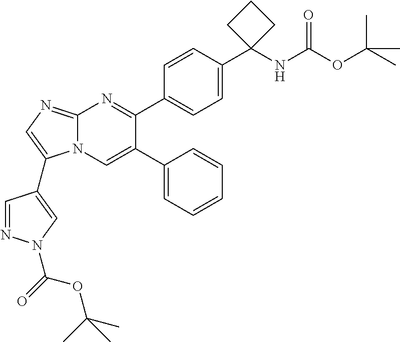 [SMILES: CC(C)(C)OC(=O)NC1(c2ccc(-c3nc4ncc(-c5cnn(C(=O)OC(C)(C)C)c5)n4cc3-c3ccccc3)cc2)CCC1] | none | RT = 1.08 min; m/z = 507 (ES+, M + 1 − Boc residue) |
| | 4-{7-[4-(1-tert.-Butoxycarbonylamino- | | |
| | cyclobutyl)-phenyl]-6-phenyl- | | |
| | imidazo[1,2-a]pyrimidin-3-yl}-pyrazole- | | |
| | 1-carboxyl acid tert.-butyl ester | | |

### Example 1-0

### 1-[4-(2-methyl-6-phenyl-[1,2,4]triazolo[1,5-a]pyrimidin-5-yl)-phenyl]-cyclobutylamine

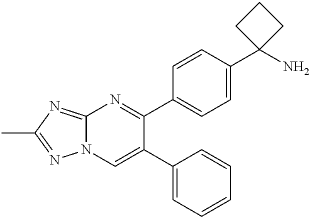 [SMILES: Cc1nc2nc(-c3ccc(C4(N)CCC4)cc3)c(-c3ccccc3)cn2n1]

A mixture of {1-[4-(2-methyl-6-phenyl-[1,2,4]triazolo[1,5-a]pyrimidin-5-yl)-phenyl]-cyclobutyl}-carbamic acid tert-butyl ester (692 mg) and a solution of 4 M hydrochloric acid in dioxane (15 mL), under nitrogen, was stirred at rt. On completion, the mixture was concentrated in vacuo, suspended in dilute aqueous sodium bicarbonate and filtered. The filtrate was extracted with DCM, dried and concentrated in vacuo. Trituration with EtOAc gave the title compound (330 mg);

UPLC-MS: RT=0.80 min; m/z=353.27 (100%, M−NH₂), 370.29 (M+1);

¹H NMR (400 MHz, d6-DMSO): δ 9.32 (s, 1H), 7.28-7.39 (m, 9H), 2.54 (s, 3H), 2.29-2.36 (m, 2H), 1.88-2.10 (m, 5H), 1.58-1.67 (m, 1H) ppm;

Melting point=206.7° C.

The residue from the aqueous work up and the mother liquors from the trituration were combined and purification was achieved by preparative reverse phase HPLC to give further title compound (95 mg).

### Example 1-1

### 1-[4-(2-methyl-6-phenyl-[1,2,4]triazolo[1,5-a]pyrimidin-5-yl)-phenyl]-cyclopentylamine

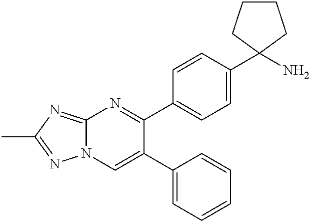 [SMILES: Cc1nc2nc(-c3ccc(C4(N)CCCC4)cc3)c(-c3ccccc3)cn2n1]

A mixture of {1-[4-(2-methyl-6-phenyl-[1,2,4]triazolo[1,5-a]pyrimidin-5-yl)-phenyl]-cyclopentyl}-carbamic acid tert-butyl ester (376 mg) and a solution of 4 M hydrochloric acid in dioxane (8 mL), under nitrogen, was stirred overnight at rt. The mixture was concentrated in vacuo, taken up in EtOAc and washed with dilute aqueous sodium bicarbonate, filtered and concentrated in vacuo. Purification was achieved by preparative reverse phase HPLC to give the title compound (76 mg) contaminated with formic acid.

UPLC-MS: RT=0.80 min; m/z=353.27 (100%, M−NH₂), 370.29 (M+1);

¹H NMR (400 MHz, d6-DMSO): δ 9.32 (s, 1H), 7.43 (d, 2H), 7.28-7.38 (m, 7H), 2.53 (s, 3H), 1.81-1.89 (m, 6H), 1.66-1.71 (m, 2H) ppm.

The following examples were prepared in analogy.

| Example | Structure/Name | 1H-NMR | UPLC-MS |
|---|---|---|---|
| 1-2 | 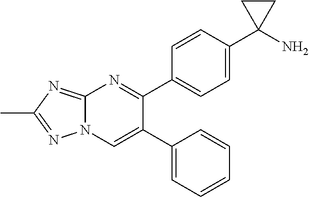 [SMILES: Cc1nc2nc(-c3ccc(C4(N)CC4)cc3)c(-c3ccccc3)cn2n1] | (300 MHz, d6-DMSO): δ 9.33 (s, 1H), 7.27- 7.38 (m, 7H), 7.23 (d, 2H), 2.54 (s, 3H), 0.98- 1.10 (m, 4H) ppm. | RT = 0.74; m/z = 342.27 (M + 1) |
| | 1-[4-(2-methyl-6-phenyl- | | |
| | [1,2,4]triazolo[1,5-a]pyrimidin- | | |
| | 5-yl)-phenyl]- | | |
| | cyclopropylamine | | |
| 1-3 | 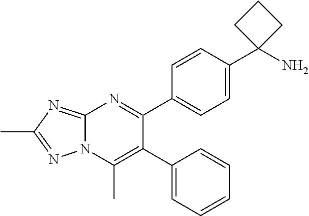 | (300 MHz, d6-DMSO): δ 7.26-7.42 (m, 9H), 2.56 (m, 6H), 2.31- 2.40 (m, 2H), 2.12- 2.20 (m, 2H), 1.93- 2.07 (m, 1H), 1.58- 1.72 (m, 1H) ppm | RT = 0.84 min; m/z = 353.54 (M − NH₂) |
| | 1-[4-(2,7-dimethyl-6-phenyl- | | |
| | [1,2,4]triazolo[1,5-a]pyrimidin- | | |
| | 5-yl)-phenyl]-cyclobutylamine | | |
| 1-4 | 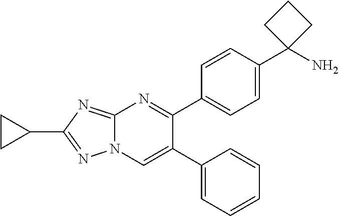 [SMILES: NC1(c2ccc(-c3nc4nc(C5CC5)nn4cc3-c3ccccc3)cc2)CCC1] | (300 MHz, d6-DMSO): δ 9.28 (s, 1H), 7.26- 7.39 (m, 9H), 2.16- 2.36 (m, 3H), 1.90- 2.10 (m, 5H), 1.56- 1.67 (m, 1H), 1.02- 1.13 (m, 4H) ppm | RT = 0.86 min; m/z = 365.21 (M − NH₂), 382.21 (M + 1) |
| | 1-[4-(2-cyclopropyl-6-phenyl- | | |
| | [1,2,4]triazolo[1,5-a]pyrimidin- | | |
| | 5-yl)-phenyl]-cyclobutylamine | | |

### Example 2-0

### 1-[4-(6-Phenyl-imidazo[1,2-a]pyrimidin-7-yl)-phenyl]-cyclobutylamine

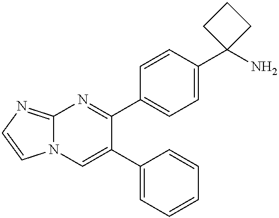 [SMILES: NC1(c2ccc(-c3nc4nccn4cc3-c3ccccc3)cc2)CCC1]

74.5 mg (0.17 mmol) {1-[4-(6-Phenyl-imidazo[1,2-a]pyrimidin-7-yl)-phenyl]-cyclobutyl}-carbamic acid tert.-butylester (intermediate example Int-2-0) are dissolved in 2.8 mL dioxane. After addition of 0.42 mL 4M hydrochloric acid in dioxane the reaction mixture is stirred for 20 hours at room temperature. The solvent is removed, the residue is brought to pH 9 with sodium bicarbonate and the mixture is stirred for one hour. The reaction mixture is extracted three times with ethyl acetate (30 mL each). The combined organic extracts are washed with water and dried over sodium sulfate. After filtration and evaporation of the solvent the residue is purified by chromatography on silicagel (eluents: dichloromethane/methanol) resulting in 3.5 mg (5.8%) of the desired compound.

MS (ES+, M+1): 341

¹H-NMR (300 MHz, CD₃OD): δ 8.86 (s, 1H), 7.87 (d, 1H), 7.78 (d, 1H), 7.20-7.48 (m, 9H), 2.42-2.59 (m, 2H), 2.12-2.29 (m, 2H), 1.98-2.12 (m, 1H), 1.65-1.80 (m, 1H).

### Example 2-1

### 1-[4-(2-Methyl-6-phenyl-imidazo[1,2-a]pyrimidin-7-yl)-phenyl]-cyclobutylamine

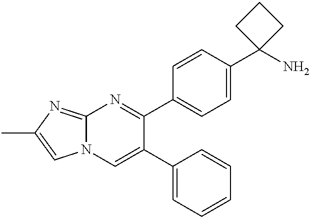 [SMILES: Cc1cn2cc(-c3ccccc3)c(-c3ccc(C4(N)CCC4)cc3)nc2n1]

185 mg (0.41 mmol) {1-[4-(2-Methyl-6-phenyl-imidazo[1,2-a]pyrimidin-7-yl)-phenyl]-cyclobutyl}-carbamic acid tert.-butylester (intermediate example Int-2-1) are dissolved 4 mL 4M hydrochloric acid in dioxane. The reaction mixture is stirred for five days at room temperature. The solvent is removed, the residue is brought to pH 9 with saturated sodium bicarbonate. The reaction mixture is extracted twice with dichloromethane (80 mL each). The combined organic extracts are washed with water and dried over sodium sulfate. After filtration and evaporation of the solvent the residue is purified by chromatography on silicagel (eluents: dichloromethane/methanol) yielding 53.1 mg (35%) of the title compound.

MS (Cl+, M+1): 355

¹H-NMR (300 MHz, d6-DMSO): δ 8.90 (s, 1H), 7.62 (s, 1H), 7.18-7.39 (m, 9H), 2. 88 (very br., 2H), 2.39 (s, 3H), 2.28-2.39 (m, 2H), 1.89-2.10 (m, 3H), 1.52-1.68 (m, 1H).

### Example 2-2

### {7-[4-(1-Amino-cyclobutyl)-phenyl]-6-phenyl-imidazo[1,2-a]pyrimidin-5-yl)-cyclopropylamine

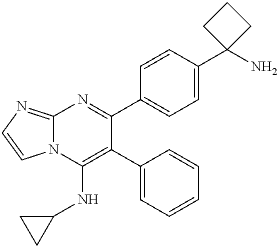 [SMILES: NC1(c2ccc(-c3nc4nccn4c(NC4CC4)c3-c3ccccc3)cc2)CCC1]

405 mg {1-[4-(5-Cyclopropylamino-6-phenyl-imidazo[1,2-a]pyrimidin-7-yl]phenyl]-cyclobutyl}-carbamic acid tert.-butylester (intermediate example Int-2-2) are stirred with 4.1 mL of a 4M hydrochloric acid in dioxane for five days at room temperature. The solvent is evaporated and the residue is treated with aqueous sodium bicarbonate solution (pH 9). After stirring for two hours 100 mL ethyl acetate are added and stirring is continued for three hours. The organic phase is separated and the aqueous phase is extracted with ethyl acetate (50 mL). The combined organic extracts are washed with water, dried, filtrated and the solvent is removed. After purification of the residue by HPLC (twice) 58 mg of the title compound are obtained.

UPLC-MS: RT=0.66 min; m/z=394 (ES−, M−1)

¹H-NMR (300 MHz, CD₃OD): δ 8.49 (br., 1H), 8.05 (d, 1H), 7.65 (d, 1H), 7.15-7.42 (m, 9H), 2.60-2.78 (m, 2H), 2.41-2.60 (m, 2H), 2.28-2.38 (m, 1H), 2.08-2.28 (m, 1H), 1.79-2.00 (m, 1H), 0.48-0.59 (m, 2H), 0.29-0.42 (m, 2H).

The following examples have been prepared in analogy according to example 2-2 by reacting the corresponding intermediates Int-2 with 4M hydrochloric acid in dioxane.

| | | | UPLC-MS |
|---|---|---|---|
| | | | (rt in min.) |
| Example | Structure/Name | 1H-NMR | resp. MS |
| 2-3 | 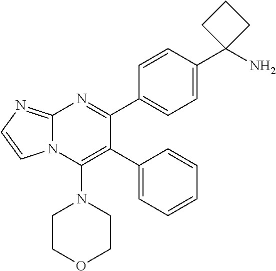 [SMILES: NC1(c2ccc(-c3nc4nccn4c(N4CCOCC4)c3-c3ccccc3)cc2)CCC1] | (400 MHz, CD₃OD): δ 7.78 (d, 1H), 7.73 (d, 1H), 7.29-7.38 (m, 3H), 7.15-7.29 (m, 6H), 3.65-3.78 (m, 4H), 2.88 (very br., 4H), 2.39-2.55 (m, 2H), 2.11-2.22 (m, 2H), 1.95-2.10 (m, 1H), 1.60-1.78 (m, 1H). | (ES+, M + 1): 426 |
| | 1-[4-(5-Morpholino-4-yl-6-phenyl- | | |
| | imidazo[1,2-a]pyrimidin-7-yl) | | |
| | phenyl]-cyclobutylamine | | |
| 2-4 | 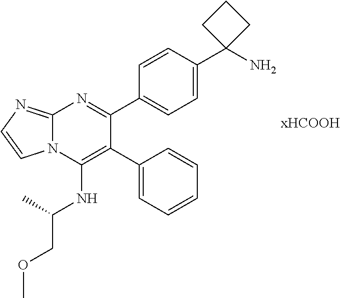 [SMILES: COC[C@H](C)Nc1c(-c2ccccc2)c(-c2ccc(C3(N)CCC3)cc2)nc2nccn12] | (400 MHz, d6-DMSO): δ 8.29 (s, 1H), 8.10 (d, 1H), 7.62 (d, 1H), 7.01-7.45 (m, 9H), 6.06 (d, 1H), 3.01- 3.30 (m, 3H), 3.00 (s, 3H), 2.22-2.39 (m, 2H), 2.06-2.21 (m, 2H), 1.89-2.05 (m, 1H), 1.53-1.69 (m, 1H), 0.87 (d, 3H). | (ES−, M − 1): 426 |
| | {7-[4-(1-Amino-cyclobutyl)-phenyl]- | | |
| | 6-phenyl-imidazo[1,2-a]pyrimidin-5- | | |
| | yl}-((S)-2-methoxy-1-methyl-ethyl)- | | |
| | amine | | |
| 2-5 | 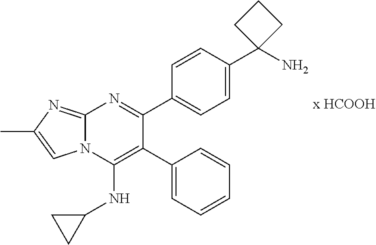 [SMILES: Cc1cn2c(NC3CC3)c(-c3ccccc3)c(-c3ccc(C4(N)CCC4)cc3)nc2n1] | (300 MHz, d6-DMSO): δ 8.23 (s, 1H), 7.80 (s, 1H), 6.88-7.40 (m, 9H), 2.10-2.45 (m with a singulet within, 8H), 1.88-2.08 (m, 1H), 1.53-1.75 (m, 1H), 0.30-49 (m, 2H), 0.08- 0.23 (m, 2H). | |
| | {7-[4-(1-Amino-cyclobutyl)-phenyl]-2- | | |
| | methyl-6-phenyl-imidazo[1,2- | | |
| | a]pyrimidin-5-yl)-cyclopropylamine | | |
| 2-6 | 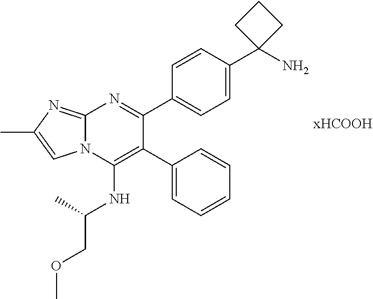 [SMILES: COC[C@H](C)Nc1c(-c2ccccc2)c(-c2ccc(C3(N)CCC3)cc2)nc2nc(C)cn12] | (400 MHz, d6-DMSO): δ 8.28 (s, 1H), 7.80 (s, 1H), 7.05-7.40 (m, 9H), 5.85 (d, 1H), 3.02-3.30 (m, 3H), 3.00 (s, 3H), 2.28-2.39 (m with a singulet within, 5H), 2.10-2.21 (m, 2H), 1.92-2.05 (m, 1H), 1.55-1.70 (m, 1H), 0.89 (d, 3H). | (ES−, M − 1): 440 |
| | {7-[4-(1-Amino-cyclobutyl)-phenyl]-2- | | |
| | methyl-6-phenyl-imidazo[1,2- | | |
| | a]pyrimidin-5-yl}-((S)-2-methoxy-1- | | |
| | methyl-ethyl)-amine | | |

### Example 3-0

### {5-[4-(1-Aminocyclobutyl)-phenyl]-6-phenyl-2-pyridine-2-yl-[1,2,4]triazolo[1,5-a]pyrimidin-7-yl}-tert.-butylamine

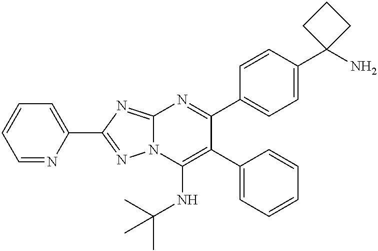 [SMILES: CC(C)(C)Nc1c(-c2ccccc2)c(-c2ccc(C3(N)CCC3)cc2)nc2nc(-c3ccccn3)nn12]

310 mg (0.53 mmol) {1-[4-(7-tert.-Butylamino-6-phenyl-2-pyridine-2-yl-[1,2,4]triazolo[1,5-a]pyrimidin-5-yl)phenyl]-cyclobutyl}-carbamic acid tert.-butyl ester (intermediate example Int-3-0) are dissolved in 14.8 mL dioxane. After addition of 14.8 mL 4M hydrochloric acid in dioxane the reaction mixture is stirred for 6 h at room temperature. The solvent is evaporated and the residue is treated with ethyl acetate and water. After stirring for two hours the reaction mixture is extracted twice with ethyl acetate. The combined organic extracts are washed with brine, dried and filtrated. The solvent is evaporated and the residue is purified on amine silicagel (eluents: dichloromethane/methanol) yielding the title compound which is however contaminated. After a further chromatography and an additional HPLC 14 mg (5.2%) of the pure title compound are obtained.

MS (ES+, M+1): 490

¹H-NMR (300 MHz, CD₃OD): δ 8.78 (d, 1H), 8.35 (d, 1H), 8.01 (dd, 1H), 7.49-7.60 (m, 1H), 7.22-7.49 (m, 9H), 2.40-2.58 (m, 2H), 2.15-2.30 (m, 2H), 1.97-2.13 (m, 1H), 1.62-1.80 (m, 1H), 1.52 (s, 9H).

The following examples have been prepared in analogy according to example 4 by reacting the corresponding intermediate examples Int-3 with 4M hydrochloric acid in dioxane.

| | | | UPLC-MS |
|---|---|---|---|
| | | | (rt in min) |
| Example | Structure/Name | 1H-NMR | resp. MS |
| 3-1 | 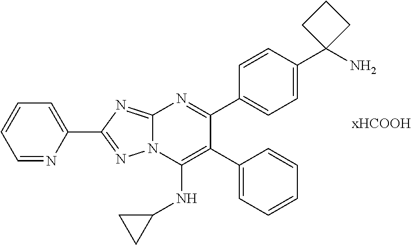 [SMILES: NC1(c2ccc(-c3nc4nc(-c5ccccn5)nn4c(NC4CC4)c3-c3ccccc3)cc2)CCC1] | (300 MHz, CD₃OD): δ 8.75 (d, 1H), 8.52 (s, 0.8H), 8.39 (d, 1H), 8.04 (dd, 1H), 7.49- 7.62 (m, 1H), 7.19- 7.45 (m, 9H), 2.54- 2.72 (m, 2H), 2.35- 2.52 (m, 2H), 2.20- 2.34 (m, 1H), 2.05- 2.20 (m, 1H), 1.75- 1.95 (m, 1H), 0.49- 0.65 (m, 2H), 0.18- 0.35 (m, 2H). | (ES+, M + 1): 474 |
| | {5-[4-(1-Aminocyclobutyl)-phenyl]-6- | | |
| | phenyl-2-pyridine-2-yl- | | |
| | [1,2,4]triazolo[1,5-a]pyrimidin-7-yl}- | | |
| | cyclopropylamine | | |
| 3-2 | 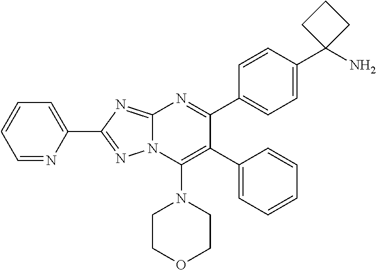 [SMILES: NC1(c2ccc(-c3nc4nc(-c5ccccn5)nn4c(N4CCOCC4)c3-c3ccccc3)cc2)CCC1] | (400 MHz, CD₃OD): δ 8.73 (d, 1H), 8.40 (d, 1H), 8.03 (dd, 1H), 7.49-7.63 (m, 1H), 7.19-7.41 (m, 9H), 3.68-3.80 (m, 4H), 3.20-3.40 (m, 4H, obscured by the signal of the solvent), 2.42-2.52 (m, 2H), 2.13-2.30 (m, 2H), 1.98-2.12 (m, 1H), 1.65-1.80 (m, 1H). | (ES+, M + 1): 504 |
| | 1-[4-(7-Morpholine-4-yl-6-phenyl-2- | | |
| | pyridine-2-yl-[1,2,4]triazolo[1,5- | | |
| | a]pyrimidin-5-yl)-phenyl]- | | |
| | cyclobutylamine | | |
| 3-3 | 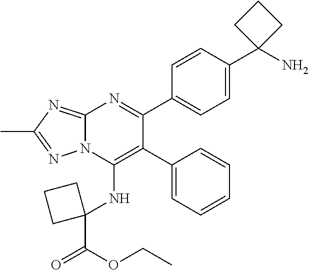 [SMILES: CCOC(=O)C1(Nc2c(-c3ccccc3)c(-c3ccc(C4(N)CCC4)cc3)nc3nc(C)nn23)CCC1] | (300 MHz, d6- DMSO): δ 7.09-7.43 (m, 9H), 4.05 (q, 2H), 2.49-2.65 (m, 2H, obscured by the signal of the solvent), 2.16-2.40 (m with a singulet within, 7H), 1.75-2.14 (m, 5H), 1.52-1.68 (m, 1H), 1.09 (t, 3H). | RT = 1.04 min; m/z = 495 (ES−, M − 1) |
| | 1-{5-[4-(1-Aminocyclobutyl)-phenyl]- | | |
| | 2-methyl-6-phenyl-[1,2,4]triazolo[1,5- | | |
| | a]pyrimidin-7-ylamino}- | | |
| | cyclobutanecarboxylic acid ethyl | | |
| | ester | | |
| 3-4 | 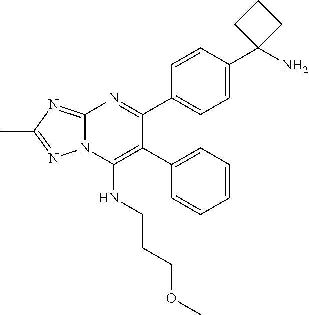 [SMILES: COCCCNc1c(-c2ccccc2)c(-c2ccc(C3(N)CCC3)cc2)nc2nc(C)nn12] | | RT = 0.80 min; m/z = 443.1 (UPLC-MS Method B) |
| | {5-[4-(1-Amino-cyclobutyl)-phenyl]- | | |
| | 2-methyl-6-phenyl-[1,2,4]triazolo | | |
| | [1,5-a]pyrimidin-7-yl}-(3-methoxy- | | |
| | propyl)-amine | | |
| 3-5 | 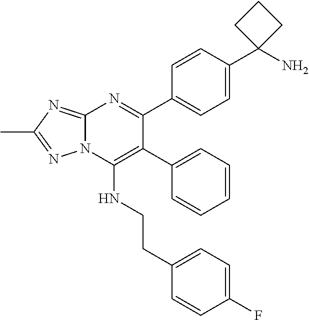 | | RT = 0.92 min; m/z = 486.1 (UPLC-MS Method B) |
| | {5-[4-(1-Amino-cyclobutyl)-phenyl]- | | |
| | 2-methyl-6-phenyl-[1,2,4]triazolo | | |
| | [1,5-a]pyrimidin-7-yl}-[2-(4-fluoro- | | |
| | phenyl)-ethyl]-amine | | |
| 3-6 | 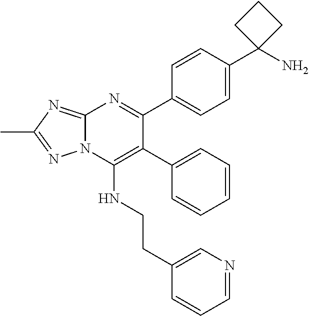 [SMILES: Cc1nc2nc(-c3ccc(C4(N)CCC4)cc3)c(-c3ccccc3)c(NCCc3cccnc3)n2n1] | | RT = 0.63 min; m/z = 476.1 (UPLC-MS Method B) |
| | {5-[4-(1-Amino-cyclobutyl)-phenyl]- | | |
| | 2-methyl-6-phenyl-[1,2,4]triazolo | | |
| | [1,5-a]pyrimidin-7-yl}-(2-pyridin-3-yl- | | |
| | ethyl)-amine | | |

### Example 4-0

### 1-{4-[3-(4-Methansulfonyl-phenyl)-6-phenyl-imidazo[1,2-a]pyrimidin-7-yl]-phenyl}-cyclobutylamine

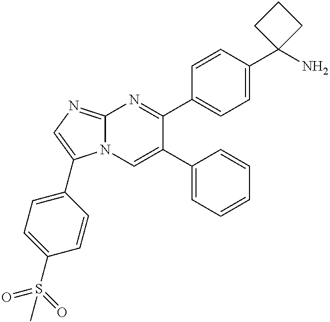 [SMILES: CS(=O)(=O)c1ccc(-c2cnc3nc(-c4ccc(C5(N)CCC5)cc4)c(-c4ccccc4)cn23)cc1]

### Step 1: {1-[4-(3-bromo-6-phenyl-imidazo[1,2-a]pyrimidin-7-yl]-phenyl}-cyclobutyl)-carbamic acid tert.-butylester

397 mg (0.9 mmol) {1-[4-(6-Phenyl-imidazo[1,2-a]pyrimidin-7-yl)-phenyl]-cyclobutyl}-carbamic acid tert.-butylester are dissolved in 7.2 mL trichloromethane. After addition of 240.6 mg (1.35 mmol) N-bromosuccinimide the reaction mixture is heated to reflux for two and a half hours. The solvent is evaporated and the residue is purified by chromatography on silicagel (eluents: dichloromethane/methanol) yielding 357.6 mg of the crude title compound which however contains a larger amount of succinimide.

MS (ES+, M+1): 519/521

¹H-NMR (300 MHz, d6-DMSO): δ 8.59 (s, 1H), 7.93 (s, 1H), 7.17-7.39 (m, 9H), 2.20-2.39 (m, 4H), 1.82-2.02 (m, 1H), 1.60-1.80 (m, 1H), 0.95-1.40 (m, 9H).

### Step 2: (1-{4-[3-(4-methanesulfonyl-phenyl)-6-phenyl-imidazo[1,2-a]pyrimidin-7-yl]-phenyl}-cyclobutyl)-carbamic acid tert.-butylester

350 mg (0.67 mmol) {1-[4-(3-Bromo-6-phenyl-imidazo[1,2-a]pyrimidin-7-yl]-phenyl}-cyclobutyl)-carbamic acid tert.-butylester, 269.5 mg (1.35 mmol) 4-(methanesulfonylphenyl)boronic acid, 58.9 mg (0.067 mmol) 1,1bis(diphenylphosphino)ferrocenedichloropalladium(II) and 214 mg (2.02 mmol) sodium carbonate in 7.2 mL dioxane and 1.3 mL water (both solvents have been degassed for 10′) are heated in a microwave vial which has been sealed with a microwave cap for 18 hours at 105° C. (heating block). The reaction mixture is poured on water (50 mL)/dichloromethane (150 mL) and stirred vigorously for 30′. The organic phase is separated and the aqueous phase is extracted once more with dichloromethane (50 mL). The combined organic extracts are washed twice with brine, dried (sodium sulfate), filtrated and the solvent is evaporated. Because the aqueous phase contains some product it is stirred in 200 mL ethyl acetate/methanol (5%) for 90 minutes. The organic phase is separated, washed with brine, dried, filtrated and the solvent is removed. The combined crude residues (384.6 mg, contaminated) are used in the next step without further purification.

UPLC-MS: RT=1.36 min; m/z=595 (ES+, M+1)

### Step 3: 1-{4-[3-(4-methansulfonyl-phenyl)-6-phenyl-imidazo[1,2-a]pyrimidin-7-yl]-phenyl}-cyclobutylamine

384.6 mg (0.65 mmol) (1-{4-[3-(4-Methanesulfonyl-phenyl)-6-phenyl-imidazo[1,2-a]pyrimidin-7-yl]-phenyl}-cyclobutyl)-carbamic acid tert.-butylester (crude) are stirred in 21.9 mL 4M hydrochloric acid in dioxane for three days at room temperature. The solvent is removed and the residue is treated with saturated sodium bicarbonate until a pH of 9. After stirring for two hours at room temperature 100 mL dichloromethane are added and stirring is continued for three hours. The organic phase is separated and the aqueous phase is extracted once more with 50 mL dichloromethane. The combined organic extracts are washed with water and brine. After drying and filtration the solvent is removed and the residue is purified by HPLC yielding 19.9 mg of the title compound.

¹H-NMR (400 MHz, d6-DMSO): δ 9.01 (s, 1H), 8.53 (s, 1H), 8.30 (d, 2H), 8.01 (d, 2H), 7.21-7.44 (m, 9H), 3.25 (s, 3H), 2.28-2.39 (m, 2H), 1.89-2.12 (m, 3H), 1.53-1.70 (m, 1H).

The following examples have been prepared in analogy according to example 4-0 by reacting {1-[4-(3-bromo-6-phenyl-imidazo[1,2-a]pyrimidin-7-yl]-phenyl}-cyclobutyl)-carbamic acid tert.-butylester with the appropriate boronic acid, cleavage of the protecting group and subsequent purification or in the case of example 4-3 by cleavage of the protecting group of {1-[4-(3-bromo-6-phenyl-imidazo[1,2-a]pyrimidin-7-yl]-phenyl}-cyclobutyl)-carbamic acid tert.-butylester and subsequent purification.

The following examples were prepared in analogy to the examples described above.

| Example | Structure/Name | 1H-NMR | UPLC-MS |
|---|---|---|---|
| 5-0 | 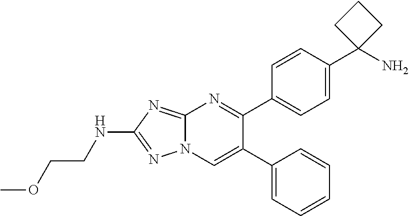 | | RT = 0.81 min; m/z = 398.60 (M − NH₂) |
| | {5-[4-(1-Amino-cyclobutyl)- | | |
| | phenyl]-6-phenyl- | | |
| | [1,2,4]triazolo[1,5-a]pyrimidin- | | |
| | 2-yl}-(2-methoxy-ethyl)-amine | | |
| 5-1 | 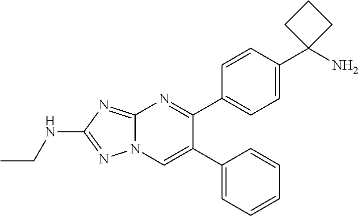 | (300 MHz, d6-DMSO): δ 9.02 (s, 1H), 7.27- 7.37 (m, 9H), 7.00 (m, 1H), [2H obscured by solvent], 2.31 (m, 2H, partially obscured by solvent), 1.95-2.08 (m), 1.63 (m, 1H), 1.19 (t, 3H) ppm | RT = 0.85 min; m/z = 368.57 (M − NH₂) |
| | {5-[4-(1-amino-cyclobutyl)- | | |
| | phenyl]-6-phenyl- | | |
| | [1,2,4]triazolo[1,5-a]pyrimidin- | | |
| | 2-yl}-ethyl-amine | | |
| 5-2 | 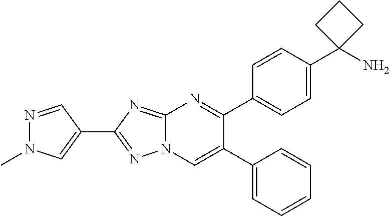 | | RT = 0.83 min; m/z = 405.53 (M − NH₂) |
| | 1-{4-[2-(1-methyl-1H-pyrazol- | | |
| | 4-yl)-6-phenyl- | | |
| | [1,2,4]triazolo[1,5-a]pyrimidin- | | |
| | 5-yl]-phenyl}-cyclobutylamine | | |
| 5-3 | 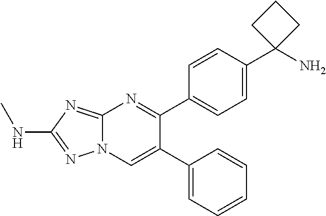 | (300 MHz, d6-DMSO): δ 9.03 (s, 1H), 7.24- 7.37 (m, 9H), 6.92 (q, 1H), 2.87 (d, 3H), 2.28- 2.37 (m, 2H), 2.15 (br s), 1.95-2.08 (m, 3H), 1.58-1.67 (m, 1H) ppm | RT = 0.76 min; m/z = 354.21 (M − NH₂) |
| | {5-[4-(1-amino-cyclobutyl)- | | |
| | phenyl]-6-phenyl- | | |
| | [1,2,4]triazolo[1,5-a]pyrimidin- | | |
| | 2-yl}-methyl-amine | | |
| 5-4 | 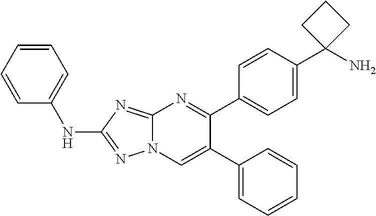 [SMILES: NC1(c2ccc(-c3nc4nc(Nc5ccccc5)nn4cc3-c3ccccc3)cc2)CCC1] | (400 MHz, d6-DMSO): δ 9.92 (s, 1H), 9.24 (s, 1H), 7.75 (d, 2H), 7.30- 7.40 (m, 11H), 6.93 (t, 1H), 2.31-2.38 (m, 2H), 1.90-2.11 (m, 3H), 1.59-1.69 (m, 1H) ppm | RT = 1.03 min; m/z = 416.57 (M − NH₂) |
| | {5-[4-(1-Amino-cyclobutyl)- | | |
| | phenyl]-6-phenyl- | | |
| | [1,2,4]triazolo[1,5-a]pyrimidin- | | |
| | 2-yl}-phenyl-amine | | |
| 5-5 | 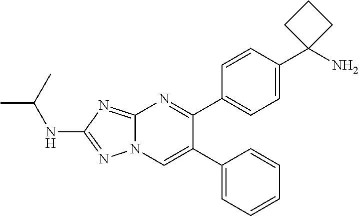 | (300 MHz, d6-DMSO): δ 9.01 (s, 1H), 7.24- 7.36 (m, 9H), 6.90 (d, 1H), 3.84-3.93 (m, 1H), 2.29-2.36 (m, 2H), 2.21 (br s), 1.93- 2.08 (m, 3H), 1.58- 1.67 (m, 1H) ppm | RT = 0.86 min; m/z = 382.23 (M − NH₂) |
| | {5-[4-(1-amino-cyclobutyl)- | | |
| | phenyl]-6-phenyl- | | |
| | [1,2,4]triazolo[1,5-a]pyrimidin- | | |
| | 2-yl}-isopropyl-amine | | |
| 5-6 | 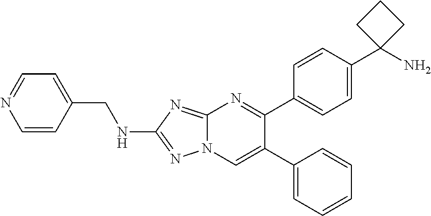 [SMILES: NC1(c2ccc(-c3nc4nc(NCc5ccncc5)nn4cc3-c3ccccc3)cc2)CCC1] | (300 MHz, d6-DMSO): δ 9.02 (s, 1H), 8.49 (d, 2H), 7.75 (t, 1H), 7.23- 7.36 (m, 11H), 4.54 (d, 2H), 2.28-2.36 (m, 2H), 1.92-2.08 (m, 3H), 1.55-1.67 (m, 1H) ppm | RT = 0.66 min; m/z = 431.30 (M − NH₂) |
| | {5-[4-(1-amino-cyclobutyl)- | | |
| | phenyl]-6-phenyl- | | |
| | [1,2,4]triazolo[1,5-a]pyrimidin- | | |
| | 2-yl}-pyridin-4-ylmethyl-amine | | |
| 5-7 | 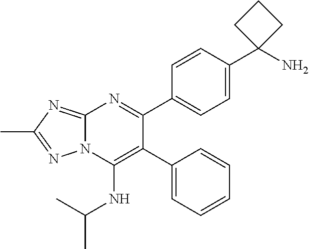 [SMILES: Cc1nc2nc(-c3ccc(C4(N)CCC4)cc3)c(-c3ccccc3)c(NC(C)C)n2n1] | (300 MHz, d6-DMSO): δ 7.28-7.36 (m, 5H), 7.23 (d, 2H), 7.13 (d, 2H), 6.58 (d, 1H), 3.57 (m, 1H), [3H, s obscured by solvent], 1.89-2.32 (m, 7H), 1.54-1.63 (m, 1H), 1.00 (d, 6H) ppm | |
| | {5-[4-(1-amino-cyclobutyl)- | | |
| | phenyl]-2-methyl-6-phenyl- | | |
| | [1,2,4]triazolo[1,5-a]pyrimidin- | | |
| | 7-yl}-isopropyl-amine | | |
| 5-8 | 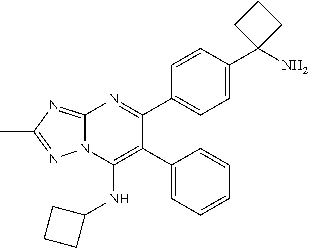 [SMILES: Cc1nc2nc(-c3ccc(C4(N)CCC4)cc3)c(-c3ccccc3)c(NC3CCC3)n2n1] | (400 MHz, d6-DMSO): δ 7.22-7.34 (m, 8H), 7.11 (m, 2H), 3.71 (m, 1H), [s, 3H obscured by solvent], 2.24- 2.30 (m, 2H), 2.12 (br s), 1.90-2.04 (m, 6H), 1.72-1.79 (m, 2H), 1.55-1.62 (m, 1H), 1.43-1.51 (m, 1H) ppm | |
| | {5-[4-(1-amino-cyclobutyl)- | | |
| | phenyl]-2-methyl-6-phenyl- | | |
| | [1,2,4]triazolo[1,5-a]pyrimidin- | | |
| | 7-yl}-cyclobutyl-amine | | |
| 5-9 | 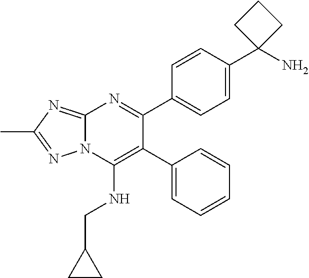 [SMILES: Cc1nc2nc(-c3ccc(C4(N)CCC4)cc3)c(-c3ccccc3)c(NCC3CC3)n2n1] | (400 MHz, d6-DMSO): δ 7.22-7.34 (m, 8H), 7.11 (d, 2H), 3.65- 3.76 (m, 1H), [s, 3H, obscured by solvent], 2.24-2.30 (m, 2H), 2.12 (br s), 1.90-2.06 (m, 6H), 1.72-1.79 (m, 2H), 1.53-1.62 (m, 1H), 1.43-1.51 (m, 1H) ppm | |
| | {5-[4-(1-amino-cyclobutyl)- | | |
| | phenyl]-2-methyl-6-phenyl- | | |
| | [1,2,4]triazolo[1,5-a]pyrimidin- | | |
| | 7-yl}-cyclopropylmethyl-amine | | |
| 5-10 | 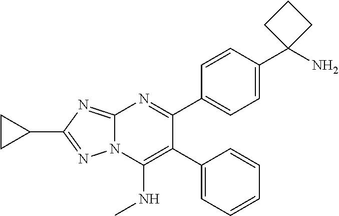 [SMILES: CNc1c(-c2ccccc2)c(-c2ccc(C3(N)CCC3)cc2)nc2nc(C3CC3)nn12] | (300 MHz, d6-DMSO): δ 7.55 (q, 1H), 7.21- 7.31 (m, 7H), 7.13 (d, 2H), [d, 3H, obscured by solvent], 2.23- 2.31 (m, 2H), 2.09- 2.18 (m, 1H), 1.92- 2.05 (m, 3H), 1.53- 1.66 (m, 1H), 1.01- 1.07 (m, 4H) ppm | RT = 0.85 min; m/z = 394.22 (M − NH₂); ES− 409.20 (M − 1) |
| | {5-[4-(1-amino-cyclobutyl)- | | |
| | phenyl]-2-cyclopropyl-6- | | |
| | phenyl-[1,2,4]triazolo[1,5- | | |
| | a]pyrimidin-7-yl}-methyl- | | |
| | amine | | |
| 5-11 | 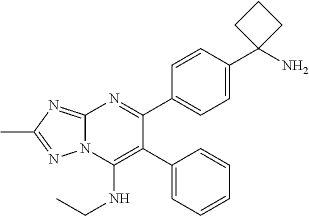 [SMILES: CCNc1c(-c2ccccc2)c(-c2ccc(C3(N)CCC3)cc2)nc2nc(C)nn12] | (300 MHz, d6-DMSO): δ 7.22-7.33 (m, 8H), 7.13 (d, 2H), 2.97- 3.06 (m, 2H), 2.48 (s, partially obscured by solvent), 2.24-2.32 (m, 2H), 1.89-2.05 (m, 3H), 1.54-1.63 (m, 1H), 0.90 (t, 3H) ppm | RT = 0.84 min; m/z = 382.21 (M − NH₂); ES− 397.23 (M − 1) |
| | {5-[4-(1-amino-cyclobutyl)- | | |
| | phenyl]-2-methyl-6-phenyl- | | |
| | [1,2,4]triazolo[1,5-a]pyrimidin- | | |
| | 7-yl}-ethyl-amine | | |
| 5-12 | 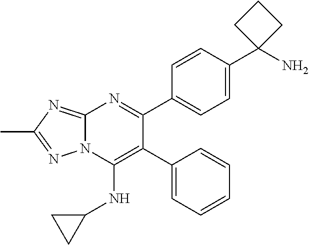 [SMILES: Cc1nc2nc(-c3ccc(C4(N)CCC4)cc3)c(-c3ccccc3)c(NC3CC3)n2n1] | (300 MHz, d6-DMSO): δ 7.75 (m, 1H), 7.22- 7.33 (m, 7H), 7.13 (d, 2H), 2.48 (s, 3H), 2.23- 2.30 (m, 2H), 1.89- 2.05 (m, 4H), 1.54- 1.63 (m, 1H), 0.48- 0.53 (m, 2H), 0.05- 0.12 (m, 2H) ppm | |
| | {5-[4-(1-amino-cyclobutyl)- | | |
| | phenyl]-2-methyl-6-phenyl- | | |
| | [1,2,4]triazolo[1,5-a]pyrimidin- | | |
| | 7-yl}-cyclopropyl-amine | | |
| 5-13 | 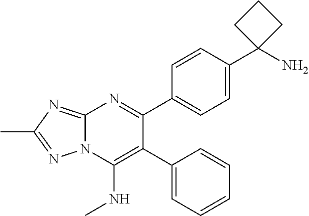 | | RT = 0.82 min; m/z = 368.22 (M − NH₂) |
| | {5-[4-(1-amino-cyclobutyl)- | | |
| | phenyl]-2-methyl-6-phenyl- | | |
| | [1,2,4]triazolo[1,5-a]pyrimidin- | | |
| | 7-yl}-methyl-amine | | |
| 5-14 | 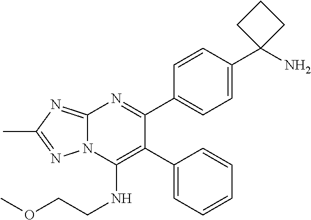 [SMILES: COCCNc1c(-c2ccccc2)c(-c2ccc(C3(N)CCC3)cc2)nc2nc(C)nn12] | | RT = 0.78 min; m/z = 429.24 (M + 1); ES− 427.21 (M − 1) |
| | {5-[4-(1-amino-cyclobutyl)- | | |
| | phenyl]-2-methyl-6-phenyl- | | |
| | [1,2,4]triazolo[1,5-a]pyrimidin- | | |
| | 7-yl}-(2-methoxy-ethyl)-amine | | |
| 5-15 | 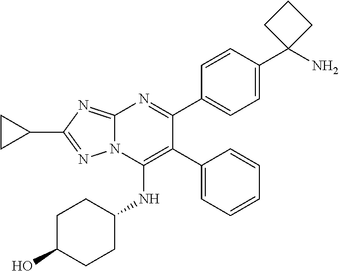 [SMILES: NC1(c2ccc(-c3nc4nc(C5CC5)nn4c(N[C@H]4CC[C@H](O)CC4)c3-c3ccccc3)cc2)CCC1] | | RT = 0.86 min; m/z = 478.59 (M − NH₂) |
| | 4-{5-[4-(1-amino-cyclobutyl)- | | |
| | phenyl]-2-cyclopropyl-6- | | |
| | phenyl-[1,2,4]triazolo[1,5- | | |
| | a]pyrimidin-7-ylamino}- | | |
| | cyclohexanol | | |

### Example 6-0

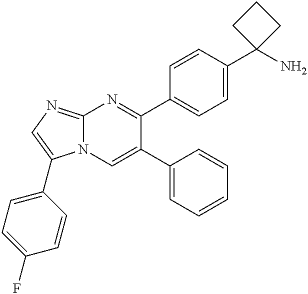 [SMILES: NC1(c2ccc(-c3nc4ncc(-c5ccc(F)cc5)n4cc3-c3ccccc3)cc2)CCC1]

### 1-{4-[3-(4-Fluorophenyl)-6-phenyl-imidazo[1,2-a]pyrimidin-7-yl]-phenyl}-cyclobutylamine

175 mg crude (1-{4-[3-(4-Fluorophenyl)-6-phenyl-imidazo[1,2-a]pyrimidin-7-yl]-phenyl}-cyclobutyl)-carbamic acid tert.-butyl ester were dissolved in 9.8 mL 4 M hydrogen chloride in dioxane. The reaction mixture was stirred for three hours at room temperature. After evaporation of the solvent the residue was dissolved in methanol and given on a PoraPak Rxn CX column. The column was washed with 100 mL methanol and the product was eluted with methanol/ammonia. The solvent was removed, and the residue was purified by HPLC yielding 44.8 mg of the title compound.

UPLC-MS: RT=0.97 min; m/z=418 (ES+, M−NH₂)

¹H-NMR (300 MHz, dDMSO): δ 8.70 (s, 1H), 8.00 (s, 1H), 7.75-7.89 (m, 2H), 7.20-7.43 (m, 11H), 2.22-2.38 (m, 2H), 2.19 (br., 2H), 1.88-2.09 (m, 3H), 1.52-1.69 (m, 1H). The following examples had been prepared in analogy according to example 6-0 by cleaving the protecting group in the corresponding intermediate examples and subsequent purification.

| | | | UPLC-MS |
|---|---|---|---|
| Example | Structure/Name | 1H-NMR | resp. MS |
| 6-1 | 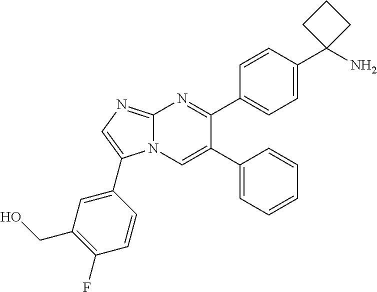 [SMILES: NC1(c2ccc(-c3nc4ncc(-c5ccc(F)c(CO)c5)n4cc3-c3ccccc3)cc2)CCC1] | (300 MHz, dDMSO): δ 8.69 (s, 1H), 7.98 (s, 1H), 7.64- 7.82 (m, 2H), 7.20-7.45 (m, 10H), 5.38 (t, 1H), 4.60 (d, 2H), 2.22-2.39 (m, 2H), 2.13 (br., 2H), 1.88-2.09 (m, 3H), 1.51-1.69 (m, 1H). | RT = 0.83 min; m/z = 448 (ES+, M − NH₂) |
| | (5-{7-[4-(1-Amino-cyclobutyl)-phenyl]-6- | | |
| | phenyl-imidazo[1,2-a]pyrimidin-3-yl}-2- | | |
| | fluoro-phenyl)-methanol | | |
| 6-2 | 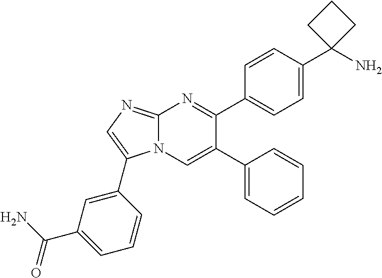 [SMILES: NC(=O)c1cccc(-c2cnc3nc(-c4ccc(C5(N)CCC5)cc4)c(-c4ccccc4)cn23)c1] | (300 MHz, dDMSO): δ 8.80 (s, 1H), 8.20 (s, 1H), 8.09 (d, 1H), 8.08 (s, 1H), 7.82- 8.00 (m, 2H), 7.53-7.68 (m, 1H), 7.49 (s, 1H), 7.22-7.42 (m, 9H), 2.22-2.38 (m, 2H), 2.19 (br., 2H), 1.88-2.10 (m, 3H), 1.52-1.69 (m, 1H). | RT = 0.77 min; m/z = 443 (ES+, M − NH₂) |
| | 3-{7-[4-(1-Amino-cyclobutyl)-phenyl]-6- | | |
| | phenyl-imidazo[1,2-a]pyrimidin-3-yl}- | | |
| | benzamide | | |
| 6-3 | 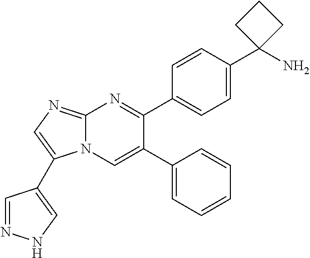 [SMILES: NC1(c2ccc(-c3nc4ncc(-c5cn[nH]c5)n4cc3-c3ccccc3)cc2)CCC1] | (300 MHz, dDMSO): 6 13.25 (br., 1H), 8.62 (s, 1H), 8.20 (very br., 2H), 7.93 (s, 1H), 7.20-7.42 (m, 9H), 2.17-2.40 (m, 4H), 1.82-2.10 (m, 3H), 1.55- 1.69 (m, 1H). | RT = 0.71 min; m/z = 390 (ES+, M − NH₂) |
| | 1-{4-[6-Phenyl-3-(1H-pyrazol-4-yl)- | | |
| | imidazo[1,2-a]pyrimidin-7-yl]-phenyl}- | | |
| | cyclobutylamine | | |

### Biological Investigations

The following assays can be used to illustrate the commercial utility of the compounds according to the present invention.

Examples were tested in selected biological assays one or more times. When tested more than once, data are reported as either average values or as median values, wherein
the average value, also referred to as the arithmetic mean value, represents the sum of the values obtained divided by the number of times tested, and
the median value represents the middle number of the group of values when ranked in ascending or descending order. If the number of values in the data set is odd, the median is the middle value. If the number of values in the data set is even, the median is the arithmetic mean of the two middle values.

Examples were synthesized one or more times. When synthesized more than once, data from biological assays represent average values or median values calculated utilizing data sets obtained from testing of one or more synthetic batch.

### Biological Assay 1.0

### Akt1 Kinase Assay

Akt1 inhibitory activity of compounds of the present invention was quantified employing the Akt1 TR-FRET assay as described in the following paragraphs.

His-tagged human recombinant kinase full-length Akt1 expressed in insect cells was purchased form Invitrogen (part number PV 3599). As substrate for the kinase reaction the biotinylated peptide biotin-Ahx-KKLNRTLSFAEPG (C-terminus in amide form) was used which can be purchased e.g. from the company Biosynthan GmbH (Berlin-Buch, Germany).

For the assay 50 nl of a 100 fold concentrated solution of the test compound in DMSO was pipetted into a black low volume 384well microtiter plate (Greiner Bio-One, Frickenhausen, Germany), 2 μl of a solution of Akt1 in assay buffer [50 mM TRIS/HCl pH 7.5, 5 mM MgCl₂, 1 mM dithiothreitol, 0.02% (v/v) Triton X-100 (Sigma)] were added and the mixture was incubated for 15 min at 22° C. to allow pre-binding of the test compounds to the enzyme before the start of the kinase reaction. Then the kinase reaction was started by the addition of 3 μl of a solution of adenosine-tri-phosphate (ATP, 16.7 μM=>final conc. in the 5 μl assay volume is 10 μM) and substrate (1.67 μM=>final conc. in the 5 μl assay volume is 1 μM) in assay buffer and the resulting mixture was incubated for a reaction time of 60 min at 22° C. The concentration of Akt1 in the assay was adjusted depending of the activity of the enzyme lot and was chosen appropriate to have the assay in the linear range, typical enzyme concentrations were in the range of about 0.05 ng/μl (final conc. in the 5 μl assay volume).

The reaction was stopped by the addition of 5 μl of a solution of HTRF detection reagents (200 nM streptavidine-XL665 [Cisbio] and 1.5 nM anti-phosho-Serine antibody [Millipore, cat. #35-001] and 0.75 nM LANCE Eu-W 1024 labeled anti-mouse IgG antibody [Perkin Elmer]) in an aqueous EDTA-solution (100 mM EDTA, 0.1% (w/v) bovine serum albumin in 50 mM HEPES/NaOH pH 7.5). The resulting mixture was incubated 1 h at 22° C. to allow the binding of the biotinylated phosphorylated peptide to the streptavidine-XL665 and the antibodies. Subsequently the amount of phosphorylated substrate was evaluated by measurement of the resonance energy transfer from the anti-mouse-IgG-Eu-Chelate to the streptavidine-XL665. Therefore, the fluorescence emissions at 620 nm and 665 nm after excitation at 350 nm was measured in a HTRF reader, e.g. a Rubystar (BMG Labtechnologies, Offenburg, Germany) or a Viewlux (Perkin-Elmer). The ratio of the emissions at 665 nm and at 622 nm was taken as the measure for the amount of phosphorylated substrate. The data were normalised (enzyme reaction without inhibitor=0% inhibition, all other assay components but no enzyme=100% inhibition). Normally test compound were tested on the same microtiter plate at 10 different concentrations in the range of 20 μM to 1 nM (20 μM, 6.7 μM, 2.2 μM, 0.74 μM, 0.25 μM, 82 nM, 27 nM, 9.2 nM, 3.1 nM and 1 nM, dilution series prepared before the assay at the level of the 100 fold conc. stock solutions by serial 1:3 dilutions) in duplicate values for each concentration and IC₅₀ values were calculated by a 4 parameter fit using an inhouse software.

### Biological Assay 2.0

### Akt2 Kinase Assay

Akt2 inhibitory activity of compounds of the present invention was quantified employing the Akt2 TR-FRET assay as described in the following paragraphs.

His-tagged human recombinant kinase full-length Akt2 expressed in insect cells and activated by PDK1 was purchased form Invitrogen (part number PV 3975). As substrate for the kinase reaction the biotinylated peptide biotin-Ahx-KKLNRTLSFAEPG (C-terminus in amide form) was used which can be purchased e.g. from the company Biosynthan GmbH (Berlin-Buch, Germany).

For the assay 50 nl of a 100 fold concentrated solution of the test compound in DMSO was pipetted into a black low volume 384well microtiter plate (Greiner Bio-One, Frickenhausen, Germany), 2 μl of a solution of Akt2 in assay buffer [50 mM TRIS/HCl pH 7.5, 5 mM MgCl₂, 1 mM dithiothreitol, 0.02% (v/v) Triton X-100 (Sigma)] were added and the mixture was incubated for 15 min at 22° C. to allow pre-binding of the test compounds to the enzyme before the start of the kinase reaction. Then the kinase reaction was started by the addition of 3 μl of a solution of adenosine-tri-phosphate (ATP, 16.7 μM=>final conc. in the 5 μl assay volume is 10 μM) and substrate (1.67 μM=>final conc. in the 5 μl assay volume is 1 μM) in assay buffer and the resulting mixture was incubated for a reaction time of 60 min at 22° C. The concentration of Akt2 in the assay was adjusted depending of the activity of the enzyme lot and was chosen appropriate to have the assay in the linear range, typical enzyme concentrations were in the range of about 0.2 ng/μl (final conc. in the 5 μl assay volume).

The reaction was stopped by the addition of 5 μl of a solution of HTRF detection reagents (200 nM streptavidine-XL665 [Cisbio] and 1.5 nM anti-phosho-Serine antibody [Millipore, cat. #35-001] and 0.75 nM LANCE Eu-W 1024 labeled anti-mouse IgG antibody [Perkin Elmer]) in an aqueous EDTA-solution (100 mM EDTA, 0.1% (w/v) bovine serum albumin in 50 mM HEPES/NaOH pH 7.5).

The resulting mixture was incubated 1 h at 22° C. to allow the binding of the biotinylated phosphorylated peptide to the streptavidine-XL665 and the antibodies.

Subsequently the amount of phosphorylated substrate was evaluated by measurement of the resonance energy transfer from the anti-mouse-IgG-Eu-Chelate to the streptavidine-XL665. Therefore, the fluorescence emissions at 620 nm and 665 nm after excitation at 350 nm was measured in a TR-FRET reader, e.g. a Rubystar (BMG Labtechnologies, Offenburg, Germany) or a Viewlux (Perkin-Elmer). The ratio of the emissions at 665 nm and at 622 nm was taken as the measure for the amount of phosphorylated substrate. The data were normalised (enzyme reaction without inhibitor=0% inhibition, all other assay components but no enzyme=100% inhibition). Normally test compound were tested on the same microtiter plate at 10 different concentrations in the range of 20 μM to 1 nM (20 μM, 6.7 μM, 2.2 μM, 0.74 μM, 0.25 μM, 82 nM, 27 nM, 9.2 nM, 3.1 nM and 1 nM, dilution series prepared before the assay at the level of the 100 fold conc. stock solutions by serial 1:3 dilutions) in duplicate values for each concentration and IC₅₀ values were calculated by a 4 parameter fit using an inhouse software.

Preferred compounds of the present invention show in either the Akt1 or Akt2 kinase assay: median IC₅₀<5 μM, more preferably, median IC₅₀<0.5 μM, even more preferably, median IC₅₀ 0.1 μM.

The following Table gives selected data for selected Examples of the present invention.

| Example | Akt1, median IC₅₀, μM | Akt2, median IC₅₀, μM |
|---|---|---|
| 1-0 | 0.794 | 0.960 |
| 1-1 | 2.574 | 3.260 |
| 1-2 | 3.013 | 2.222 |
| 1-3 | 0.526 | 1.022 |
| 1-4 | 0.755 | 0.408 |
| 2-0 | 2.223 | 1.639 |
| 2-1 | 0.474 | 0.487 |
| 2-2 | 2.046 | 1.583 |
| 2-3 | 13.106 | 7.471 |
| 2-4 | 10.630 | 1.270 |
| 2-5 | not tested | 0.540 |
| 2-6 | 13.012 | 2.864 |
| 3-0 | 1.126 | 0.061 |
| 3-1 | 2.383 | 2.319 |
| 3-2 | 20.000 | 6.637 |
| 3-3 | 20.000 | 1.121 |
| 3-4 | 18.195 | 9.345 |
| 3-5 | 18.839 | 3.557 |
| 4-0 | 0.329 | 0.299 |
| 5-0 | 1.834 | 0.877 |
| 5-1 | 0.958 | 0.502 |
| 5-2 | 0.376 | 0.340 |
| 5-3 | 0.527 | 0.381 |
| 5-4 | 0.283 | 0.197 |
| 5-5 | 1.467 | 0.234 |
| 5-6 | 5.650 | 2.548 |
| 5-7 | 0.672 | 0.549 |
| 5-8 | 0.731 | 0.303 |
| 5-9 | 0.854 | 0.541 |
| 5-10 | 0.930 | 0.982 |
| 5-11 | 1.731 | 1.120 |
| 5-12 | 1.853 | 0.904 |
| 5-13 | 2.243 | 2.240 |
| 5-14 | 3.246 | 2.319 |
| 5-15 | 11.579 | 0.936 |
| 6-0 | 1.415 | 0.681 |
| 6-1 | not tested | 0.629 |
| 6-2 | 0.824 | 0.205 |
| 6-3 | 0.235 | 0.165 |

### Cellular Assays 3.0

### p-AKT1/2/3-S473, -T308, and p-4E-BP1-T70 Assays

The molecular mechanism of action was investigated in a set of experiments to assess the inhibition of the PI3K-AKT-mTOR pathway in responsive cell lines such as KPL4 breast tumour cell line (PIK3CAH1047R, HER20/E and hormone independent). The phospho-substrates of PI3K-AKT-mTOR axis were used as the read-outs to reflect pathway inhibition. Cells were seeded at 60-80% confluency per well in 96-well cell culture plates. After overnight incubation at 37° C. 5% CO2, cells were treated with compounds and vehicle at 37° C. for 2 hours. Thereafter, cells were lysed in 150 μl lysis buffer and the levels of phospho-AKT at T308 and S473 and p-4E-BP1 at T70 sites were determined with the corresponding AlphaScreen® SureFire® assay kits (Perkin Elmer: 4E-BP1 Assay Kit Cat # TRG4E2S10K; Akt 1/2/3 p-Ser 473 #TGRA4S500 and Akt 1/2/3 p-Thr 308 #TGRA3S500 as well as IgG detection Kit #6760617M) as described in the manuals. All measurements where at least done in duplicates and confirmed by independent repetition.

Alternatively pAKT-S473 was measured using the “Akt Duplex” of the MULTI-SPOT® Assay System (Fa. Meso Scale Discovery, Cat#N41100B-1) following manufacturers instructions. Each assay used 20 μg of protein extract and measured total AKT and p-AKT content simultaneously in one well. All measurements where at least done in duplicates and confirmed by independent repetition. Values for P-AKT are expressed as percentage of P-AKT level compared to total-AKT content of the extracts.

The following Table gives selected data for selected Examples of the present invention.

| Example | pAKT-S743 median IC₅₀, μM |
|---|---|
| 1-0 | not tested |
| 1-1 | not tested |
| 1-2 | not tested |
| 1-3 | not tested |
| 1-4 | not tested |
| 2-0 | not tested |
| 2-1 | not tested |
| 2-2 | not tested |
| 2-3 | not tested |
| 2-4 | not tested |
| 2-5 | not tested |
| 2-6 | not tested |
| 3-0 | not tested |
| 3-1 | not tested |
| 3-2 | not tested |
| 3-3 | not tested |
| 3-4 | not tested |
| 3-5 | not tested |
| 4-0 | 0.23 |
| 5-0 | not tested |
| 5-1 | not tested |
| 5-2 | 0.13 |
| 5-3 | not tested |
| 5-4 | not tested |
| 5-5 | not tested |
| 5-6 | not tested |
| 5-7 | not tested |
| 5-8 | not tested |
| 5-9 | not tested |
| 5-10 | not tested |
| 5-11 | not tested |
| 5-12 | not tested |
| 5-13 | not tested |
| 5-14 | not tested |
| 5-15 | not tested |
| 6-0 | 0.92 |
| 6-1 | 0.54 |
| 6-2 | 1.15 |
| 6-3 | 0.24 |

### Biological Assay 4.0

### Tumor Cell Proliferation Assays

Compounds were tested in a cell-based assay that measures the capacity of the compounds to inhibit tumour cell proliferation following a 72 h drug exposure. Cell viability is determined using CellTiter-Glow® (CTG, Promega, cat #G7571/2/3). The CellTiter-Glo® Luminescent Cell Viability Assay is a homogeneous method to determine the number of viable cells in culture. Detection is based on using the luciferase reaction to measure the amount of ATP from viable cells. The amount of ATP in cells correlates with cell viability. Within minutes after a loss of membrane integrity, cells lose the ability to synthesize ATP, and endogenous ATPases destroy any remaining ATP; thus the levels of ATP fall precipitously.

Cells were plated at 3000-5000 cells/well (depending on the cell lines) in 90 μL growth medium on MTPs (Corning; #3603, black plate, clear flat bottom). For each cell line assayed, cells were plated onto a separate plate for determination of fluorescence at t=0 hour and t=72 hour time points. Following overnight incubation at 37° C., chemiluminescence values for the t=0 samples were determined after adding 10 μl medium and 100 μl CTG solution according to manufacture protocol. Plates for the t=72 hour time points were treated with compounds diluted into growth medium at ten times final concentration added in 10 μL to the cell culture plate. Cells were then incubated for 72 hours at 37° C. Chemiluminescence values for the t=72 hour samples were determined. For data analysis, briefly, data from 24 h plate where used to reflect 100% inhibition of growth (“Ci”) and DMSO control for uninhibited growth (“CO”) and analyzed using MTS software package for IC₅₀ and Hill coefficient. Experiments were controlled using a reference compound as standard.

Preferred compounds of the present invention show in this assay an inhibition of cell growth of cell lines such as the KPL-4 breast cancer cell line with a median IC₅₀ of <10 μM, more preferably, median IC50≦1 μM.

The following Table gives selected data for selected Examples of the present invention.

| Example | KPL-4 proliferation IC₅₀, μM |
|---|---|
| 1-0 | 3.22 |
| 1-1 | not tested |
| 1-2 | not tested |
| 1-3 | not tested |
| 1-4 | 1.41 |
| 2-0 | not tested |
| 2-1 | 1.97 |
| 2-2 | not tested |
| 2-3 | not tested |
| 2-4 | not tested |
| 2-5 | 3.62 |
| 2-6 | not tested |
| 3-0 | 1.74 |
| 3-1 | not tested |
| 3-2 | not tested |
| 3-3 | not tested |
| 3-4 | not tested |
| 3-5 | not tested |
| 4-0 | 1.27 |
| 5-0 | not tested |
| 5-1 | not tested |
| 5-2 | 0.93 |
| 5-3 | 2.17 |
| 5-4 | 6.19 |
| 5-5 | 2.20 |
| 5-6 | not tested |
| 5-7 | not tested |
| 5-8 | 3.64 |
| 5-9 | not tested |
| 5-10 | not tested |
| 5-11 | not tested |
| 5-12 | not tested |
| 5-13 | not tested |
| 5-14 | not tested |
| 5-15 | not tested |
| 6-0 | 2.54 |
| 6-1 | 2.10 |
| 6-2 | 10.00 |
| 6-3 | 5.11 |

### Biological Example 5.0

### Caco2 Permeability Assay

Caco-2 cells (purchased from DSMZ Braunschweig, Germany) were seeded at a density of 4.5×10⁴ cell per well on 24 well insert plates, 0.4 μm pore size, and grown for 15 days in DMEM medium supplemented with 10% fetal bovine serum, 1% GlutaMAX (100×, GIBCO), 100 U/ml penicillin, 100 μg/ml streptomycin (GIBCO) and 1% non essential amino acids (100×). Cells were maintained at 37° C. in a hunified 5% CO₂ atmosphere. Medium was changed every 2-3 day. Before running the permeation assay, the culture medium was replaced by a FCS-free hepes-carbonate transport puffer (pH 7.2) For assessment of monolayer integrity the transepithelial electrical resistance (TEER) was measured. Test compounds were predissolved in DMSO and added either to the apical or basolateral compartment in final concentration of 2 μM. Before and after 2 h incubation at 37° C. samples were taken from both compartments. Analysis of compound content was done after precipitation with methanol by LC/MS/MS analysis. Permeability (Papp) was calculated in the apical to basolateral (A→B) and basolateral to apical (B→A) directions. The apparent permeability was calculated using following equation:

Pₐₚₚ=(Vᵣ/Pₒ)(1/S)(P₂/t)

Where Vᵣ is the volume of medium in the receiver chamber, Pₒ is the measured peak area of the test drug in the donor chamber at t=0, S the surface area of the monolayer, P₂ is the measured peak area of the test drug in the acceptor chamber after 2 h of incubation, and t is the incubation time. The efflux ratio basolateral (B) to apical (A) was calculated by dividing the Pₐₚₚ B−A by the PₐₚₚA−B. In addition the compound recovery was calculated. As assay control reference compounds were analyzed in parallel.

### Biological Example 6.0

### In Vivo Rat Pharmacokinetics

For in vivo pharmacokinetic experiments test compounds were administered to male Wistar rats intravenously at doses of 0.5 to 1 mg/kg and intragastral at doses of 1 to 10 mg/kg formulated as solutions using solubilizers such as PEG400 in well-tolerated amounts.

For pharmacokinetics after intravenous administration test compounds were given as i.v. bolus and blood samples were taken at 2 min, 8 min, 15 min, 30 min, 45 min, 1 h, 2 h, 4 h, 6 h, 8 h and 24 h after dosing. Depending on the expected half-life additional samples were taken at later time points (e.g. 48 h, 72 h). For pharmacokinetics after intragastral administration test compounds were given intragastral to fasted rats and blood samples were taken at 5 min, 15 min, 30 min, 45 min, 1 h, 2 h, 4 h, 6 h, 8 h and 24 h after dosing. Depending on the expected half-life additional samples were taken at later time points (e.g. 48 h, 72 h). Blood was collected into Lithium-Heparintubes (Monovetten®, Sarstedt) and centrifuged for 15 min at 3000 rpm. An aliquot of 100 μL from the supernatant (plasma) was taken and precipitated by addition of 400 μL cold acetonitrile and frozen at −20° C. over night. Samples were subsequently thawed and centrifuged at 3000 rpm, 4° C. for 20 minutes. Aliquots of the supernatants were taken for analytical testing using an Agilent 1200 HPLC-system with LCMS/MS detection. PK parameters were calculated by non-compartmental analysis using a PK calculation software.

PK parameters derived from concentration-time profiles after i.v.: CLplasma: Total plasma clearance of test compound (in L/kg/h); CLblood: Total blood clearance of test compound: CLplasma*Cp/Cb (in L/kg/h) with Cp/Cb being the ratio of concentrations in plasma and blood. PK parameters calculated from concentration time profiles after i.g.: Cmax: Maximal plasma concentration (in mg/L); Cmaxnorm: Cmax divided by the administered dose (in kg/L); Tmax: Time point at which Cmax was observed (in h). Parameters calculated from both, i.v. and i.g. concentration-time profiles: AUCnorm: Area under the concentration-time curve from t=0 h to infinity (extrapolated) divided by the administered dose (in kg*h/L); AUC(0−tlast)norm: Area under the concentration-time curve from t=Oh to the last time point for which plasma concentrations could be measured divided by the administered dose (in kg*h/L); t1/2: terminal half-life (in h); F: oral bioavailability: AUCnorm after intragastral administration divided by AUCnorm after intravenous administration (in %).

The person skilled in the art will be aware of methods to show in vivo efficacy of anti-cancer compounds. By way of illustration, the following example describes methods of quantifying the in vivo efficacy in a mouse xenograft model. The skilled person will be able to apply such principles to derive models from alternative tumor material.

### Biological Example 7.0

### In Vivo Xenograft Mechanism of Action Study

To demonstrate that compounds act in tumours by the anticipated mode of action phosphorylation of the AKT protein was investigated in PC3 prostate tumours treated once with 50 mg/kg compound.

To this extent PC3 human prostate tumours were xenografted onto athymic nude mice. PC3 tumour cells were cultivated according to ATCC protocols in recommended media contained 10% FCS and harvested for transplantation in a subconfluent (70%) state. 3×10⁶ tumour cells suspended in 50% Matrigel were subcutaneously implantated into the inguinal region of male mice. Tumours were allowed to grow to the predetermined size of 60-80 mm². When the tumours were approximately in size, the animals were randomized to treatment and control groups (groups size: 9 animals) and treatment was started. Animals were treated once with 50 mg/kg compound or vehicle per oral administration (p.o.) carried out via a gastric tube. Treatment of each animal was based on individual body weight. At 2, 5 and 24 hours post treatment 3 animals each were sacrificed and the PC3 tumours excised. Tumour samples of approximately 5×5×5 mm were lysed on ice in MSD lysis buffer in the presence of protease and phosphatase inhibitors using Tissue Lyzer (Qiagen, Germany). The levels of p-AKT S473 in extracts from tumour tissue were analysed in an ELISA based assay. This assay is based on the “Akt Duplex” of the MULTI-SPOT® Assay System (Fa. Meso Scale Discovery, Cat #N41100B-1) following manufacturers instructions. Each assay used 20 μg of protein extract and measured total AKT and p-AKT content simultaneously in one well. All measurements where at least done in duplicates and confirmed by independent repetition.

Values for P-AKT are expressed as percentage of P-AKT level compared to total-AKT content of the extracts. Vehicle treated tumours were analyzed to determine the basal level of P-AKT in this model and used as a normalization control to determine the % P-AKT relative to vehicle levels.

Preferred compounds of the present invention show in this assay: relative to vehicle levels P-AKT <30% at 2 hours post treatment, more preferably at 5 hours post treatment, even more preferably at 24 hours post treatment.

The following Table gives selected data for selected Examples of the present invention.

| | P-AKT % relative to |
|---|---|
| Example | control at 5 hours |
| 1-0 | 3.2 |
| 2-1 | 6.7 |
| 5-2 | 17.9 |

### Biological Example 7.1

### In Vivo Xenograft Efficacy Study

To determine the therapeutic efficacy and tolerability of compounds, tumour growth of PC3 prostate tumours xenografted onto nude mice may be observed. Mice were treated either with vehicle or compounds.

To this extent PC3 xenografts were established as described above. Tumours were allowed to grow to the predetermined size of 25-35 mm². When the tumours were approximately in size, the animals were randomized to treatment and control groups (groups size: 8 animals) and treatment was started. Treatment of each animal was based on individual body weight and oral administration (p.o.) was carried out via a gastric tube. The oral application volumes were 10 ml/kg for mice. Mice were treated once daily with 50 mg/kg compounds.

Tumour response was assessed by determination of the tumour area (product of the longest diameter and its perpendicular) using a calliper. The animal body weight was monitored as a measure for treatment-related toxicity. Measurement of tumour area and body weight were performed 2-3 times weekly. Statistical analysis was assessed using the SigmaStat software. A one way analysis of variance was performed, and differences to the control were compared by a pair-wise comparison procedure (Dunn's method). T/C ratios (Treatment/Control) were calculated with final tumour weights at study end.

1. A compound of formula (I) 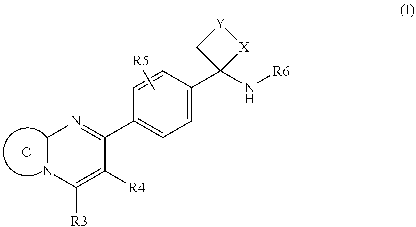
in which ring C and the pyrimidine to which it is fused form a ring system selected from 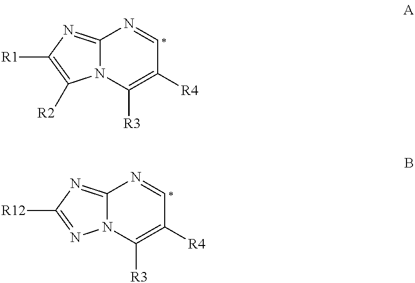
wherein * marks the point of the attachment,
wherein
R1 is hydrogen, or a group selected from 1-6C-alkyl, 3-7C-cycloalkyl, aryl, heteroaryl,
wherein said group being optionally substituted, one or more times, identically or differently, with a substituent selected from:
hydroxy, halogen, 1-6C-alkyl, 1-4C-haloalkyl, 1-6C-alkoxy, —NR8R9, cyano, —C(O)NR8R9, —C(O)OR10, —NHC(O)R11, —NHS(O)₂R11,
R12 is hydrogen, halogen, —NR13R14, or a group selected from 1-6C-alkyl, 3-7C-cycloalkyl, aryl, heteroaryl,
wherein said group being optionally substituted, one or more times, identically or differently, with a substituent selected from:
hydroxy, halogen, 1-6C-alkyl, 1-4C-haloalkyl, 1-6C-alkoxy, —NR8R9, cyano, —C(O)NR8R9, —C(O)OR10, —NHC(O)R11, —NHS(O)₂R11,
R2 is hydrogen, halogen, cyano, or a group selected from 1-6C-alkyl, 3-7C-cycloalkyl, aryl, heteroaryl, (1-6C-alkylen)-aryl, (1-6C-alkylen)-heteroaryl,
wherein said group being optionally substituted, one or more times, identically or differently, with a substituent selected from:
hydroxy, halogen, 1-6C-alkyl, 1-4C-haloalkyl, 1-6C-hydroxyalkyl, 1-6C-alkoxy, —NR8R9, cyano, —C(O)NR8R9, —C(O)OR10, —NHC(O)R11, —NHS(O)₂R11, —S(O)₂R11, —S(O)₂NR8R9,
R3 is hydrogen, 1-6C-alkyl, 3-7C-cycloalkyl, or NR15R16,
R4 is phenyl optionally substituted, one or more times, identically or differently, with a substituent selected from: 1-6C-alkyl, halogen, cyano,
R5 is hydrogen, halogen,
R6 is hydrogen, 1-6C-alkyl,
R8, R9 which can be the same or different, is
hydrogen,
1-4C-alkyl (optionally substituted in the same way or differently one or more times with halogen, hydroxy, mono- or di-(1-4C-alkylamino), 1-4C-alkoxy); or 3-7C-cycloalkyl,
or,
in the case of —NR8R9, R8 and R9 together with the nitrogen to which they are attached may also form a 3-6C-heterocyclic ring,
R10 is hydrogen, 1-6C-alkyl,
R11 is 1-4C-alkyl (optionally substituted in the same way of differently one or more times with halogen, hydroxy) or 3-7C-cycloalkyl,
R13, R14 which can be the same or different, is hydrogen, or a group selected from 1-6C-alkyl, 3-7C-cycloalkyl, aryl, heteroaryl, (1-6C-alkylen)-aryl, (1-6C-alkylen)-heteroaryl,
wherein said group being optionally substituted, one or more times, identically or differently, with a substituent selected from:
hydroxy, halogen, 1-6C-alkyl, 1-4C-haloalkyl, 1-6C-alkoxy, cyano,
R15, R16 which can be the same or different, is hydrogen, or a group selected from 1-6C-alkyl, 3-7C-cycloalkyl, (1-6C-alkylen)-aryl, (1-6C-alkylen)-heteroaryl,
wherein said group being optionally substituted, one or more times, identically or differently, with a substituent selected from:
hydroxy, halogen, 1-6C-alkyl, 1-4C-haloalkyl, 1-6C-alkoxy, cyano, 3-7-cycloalkyl, heterocyclyl, —C(O)NR10R11, —C(O)OR10, —NHC(O)R11, —NHS(O)₂R11,
or,
R15 and R16 together with the nitrogen to which they are attached may also form a 3-6C-heterocyclic ring,
X is —(CH₂)ₙ—,
n is 0, 1, 2, or 3,
Y is —CH₂—, —CH(OH)—,
or an N-oxide, a salt, a tautomer or a stereoisomer of said compound, or a salt of said N-oxide, tautomer or stereoisomer.

2. The compound of formula (I) according to claim 1
wherein
R1 is hydrogen, or a group selected from 1-6C-alkyl, 3-7C-cycloalkyl, aryl, heteroaryl,
wherein said group being optionally substituted, one or more times, identically or differently, with a substituent selected from:
hydroxy, halogen, 1-6C-alkyl, 1-4C-haloalkyl, 1-6C-alkoxy, —NR8R9, cyano, —C(O)NR8R9, —C(O)OR10, —NHC(O)R11, —NHS(O)₂R11,
R12 is hydrogen, halogen, —NR13R14, or a group selected from 1-6C-alkyl, 3-7C-cycloalkyl, aryl, heteroaryl,
wherein said group being optionally substituted, one or more times, identically or differently, with a substituent selected from:
hydroxy, halogen, 1-6C-alkyl, 1-4C-haloalkyl, 1-6C-alkoxy, —NR8R9, cyano, —C(O)NR8R9, —C(O)OR10, —NHC(O)R11, —NHS(O)₂R11,
R2 is hydrogen, halogen, cyano, or a group selected from 1-6C-alkyl, 3-7C-cycloalkyl, aryl, heteroaryl, (1-6C-alkylen)-aryl, (1-6C-alkylen)-heteroaryl,
wherein said group being optionally substituted, one or more times, identically or differently, with a substituent selected from:
hydroxy, halogen, 1-6C-alkyl, 1-4C-haloalkyl, 1-6C-hydroxyalkyl, 1-6C-alkoxy, —NR8R9, cyano, —C(O)NR8R9, —C(O)OR10, —NHC(O)R11, —NHS(O)₂R11, —S(O)₂R11, —S(O)₂NR8R9,
R3 is hydrogen, 1-6C-alkyl, 3-7C-cycloalkyl, or NR15R16,
R4 is phenyl optionally substituted, one or more times, identically or differently, with a substituent selected from: 1-6C-alkyl, halogen, cyano,
R5 is hydrogen, halogen,
R6 is hydrogen,
R8, R9 which can be the same or different, is hydrogen, 1-4C-alkyl (optionally substituted in the same way of differently one or more times with halogen, hydroxy, mono- or di-1-4C-alkylamino), 1-4C-alkoxy); or 3-7C-cycloalkyl, or, in the case of —NR8R9, R8 and R9 together with the nitrogen to which they are attached may also form a 3-6C-heterocyclic ring,
R10 is hydrogen, 1-6C-alkyl,
R11 is 1-4C-alkyl (optionally substituted in the same way of differently one or more times with halogen, hydroxy) or 3-7C-cycloalkyl,
R13, R14 which can be the same or different, is hydrogen, or a group selected from 1-6C-alkyl, 3-7C-cycloalkyl, aryl, heteroaryl, (1-6C-alkylen)-aryl, (1-6C-alkylen)-heteroaryl,
wherein said group being optionally substituted, one or more times, identically or differently, with a substituent selected from:
hydroxy, halogen, 1-6C-alkyl, 1-4C-haloalkyl, 1-6C-alkoxy, cyano,
R15, R16 which can be the same or different, is hydrogen, or a group selected from 1-6C-alkyl, 3-7C-cycloalkyl, (1-6C-alkylen)-aryl, (1-6C-alkylen)-heteroaryl,
wherein said group being optionally substituted, one or more times, identically or differently, with a substituent selected from:
hydroxy, halogen, 1-6C-alkyl, 1-4C-haloalkyl, 1-6C-alkoxy, cyano, 3-7-cycloalkyl, heterocyclyl, —C(O)OR10, or
R15, R16 together with the nitrogen atom to which they are attached may also form a 5- or 6 membered heterocyclic ring optionally containing an additional nitrogen- or oxygen atom,
X is —(CH₂)ₙ—,
n is 0, 1, 2, or 3,
Y is —CH₂—, —CH(OH)—,
or an N-oxide, a salt, a tautomer or a stereoisomer of said compound, or a salt of said N-oxide, tautomer or stereoisomer. or an N-oxide, a salt, a tautomer or a stereoisomer of said compound, or a salt of said N-oxide, tautomer or stereoisomer.

3. The compound according to claim 1,
wherein
R1 is hydrogen, or a group selected from 1-6C-alkyl, 3-7C-cycloalkyl, aryl, heteroaryl,
wherein said group being optionally substituted, one or more times, identically or differently, with a substituent selected from:
hydroxy, halogen, 1-6C-alkyl, 1-4C-haloalkyl, 1-6C-alkoxy, —NR8R9, cyano, —C(O)NR8R9, —C(O)OR10, —NHC(O)R11, —NHS(O)₂R11,
R12 is hydrogen, halogen, —NR13R14, or a group selected from 1-6C-alkyl, 3-7C-cycloalkyl, aryl, heteroaryl,
wherein said group being optionally substituted, one or more times, identically or differently, with a substituent selected from:
hydroxy, halogen, 1-6C-alkyl, 1-4C-haloalkyl, 1-6C-alkoxy, —NR8R9, cyano, —C(O)NR8R9, —C(O)OR10, —NHC(O)R11, —NHS(O)₂R11,
R2 is hydrogen, halogen, cyano, or a group selected from 1-6C-alkyl, 3-7C-cycloalkyl, aryl, heteroaryl, (1-6C-alkylen)-aryl, (1-6C-alkylen)-heteroaryl,
wherein said group being optionally substituted, one or more times, identically or differently, with a substituent selected from:
hydroxy, halogen, 1-6C-alkyl, 1-4C-haloalkyl, 1-6C-hydroxyalkyl, 1-6C-alkoxy, —NR8R9, cyano, —C(O)NR8R9, —C(O)OR10, —NHC(O)R11, —NHS(O)₂R11, —S(O)₂R11, —S(O)₂NR8R9,
R3 is hydrogen, 1-6C-alkyl, 3-7C-cycloalkyl, or NR15R16,
R4 is phenyl,
R5 is hydrogen,
R6 is hydrogen,
R8, R9 which can be the same or different, is hydrogen, 1-4C-alkyl (optionally substituted in the same way or differently one or more times with halogen, hydroxy, mono- or di-(1-4C-alkylamino), 1-4C-alkoxy); or 3-7C-cycloalkyl,
or,
in the case of —NR8R9, R8 and R9 together with the nitrogen to which they are attached may also form a 3-6C-heterocyclic ring,
R10 is hydrogen, 1-6C-alkyl,
R11 is 1-4C-alkyl (optionally substituted in the same way of differently one or more times with halogen, hydroxy) or 3-7C-cycloalkyl, R13, R14 which can be the same or different, is hydrogen, or a group selected from 1-6C-alkyl, 3-7C-cycloalkyl, aryl, heteroaryl, (1-6C-alkylen)-aryl, (1-6C-alkylen)-heteroaryl,
wherein said group being optionally substituted, one or more times, identically or differently, with a substituent selected from:
hydroxy, halogen, 1-6C-alkyl, 1-4C-haloalkyl, 1-6C-alkoxy, cyano,
R15, R16 which can be the same or different, is hydrogen, or a group selected from 1-6C-alkyl, 3-7C-cycloalkyl, (1-6C-alkylen)-aryl, (1-6C-alkylen)-heteroaryl,
wherein said group being optionally substituted, one or more times, identically or differently, with a substituent selected from:
hydroxy, halogen, 1-6C-alkyl, 1-4C-haloalkyl, 1-6C-alkoxy, cyano, 3-7-cycloalkyl, heterocyclyl, —C(O)OR10, or
or R15, R16 together with the nitrogen atom to which they are attached may also form a 5- or 6-membered heterocyclic ring optionally containing an additional nitrogen- or oxygen atom,
X is —(CH₂)ₙ—,
n is 0, 1 or 2,
Y is —CH₂—, —CH(OH)—,
or an N-oxide, a salt, a tautomer or a stereoisomer of said compound, or a salt of said N-oxide, tautomer or stereoisomer.

4. The compound according to claim 1,
wherein
R1 is hydrogen, 1-6C-alkyl,
R12 is hydrogen, 1-6C-alkyl, 3-7C-cycloalkyl, heteroaryl, NR13R14,
R2 is hydrogen, aryl, heteroaryl
wherein said aryl being optionally substituted, one or more times, identically or differently, with a substituent selected from:
halogen, 1-6C-hydroxyalkyl, cyano, —S(O)₂R11, C(O)NR8R9,
R3 is hydrogen, 1-6C-alkyl, NR15R16,
R4 is phenyl,
R5 is hydrogen,
R6 is hydrogen,
R8, R9 is hydrogen,
R10 is hydrogen, 1-4C-alkyl,
R11 is 1-4C-alkyl,
R13, R14 which can be the same or different, is hydrogen, or a group selected from 1-6C-alkyl, aryl, (1-6C-alkylen)-heteroaryl, wherein said group is optionally substituted with 1-6C-alkoxy,
R15, R16 which can be the same or different, is hydrogen, or a group selected from 1-6C-alkyl, 3-7C-cycloalkyl, (1-6C-alkylen)-aryl,
wherein said group being optionally substituted, one or more times, identically or differently, with a substituent selected from:
hydroxy, halogen, 1-6C-alkoxy, 3-7C-cycloalkyl, —C(O)OR10,
or R15, R16 together with the nitrogen atom to which they are attached may also form a 6-membered ring containing one oxygen atom,
X is —(CH₂)ₙ—,
n is 0, 1 or 2,
Y is —CH₂—,
or an N-oxide, a salt, a tautomer or a stereoisomer of said compound, or a salt of said N-oxide, tautomer or stereoisomer.

5. The compound according to claim 1,
wherein
R1 is hydrogen, methyl,
R12 is hydrogen, methyl, cyclopropyl, N-methyl-pyrazolyl, pyridyl, NR13R14,
R2 is hydrogen, 1H-pyrazol-yl, or phenyl substituted one or more times with fluorine, cyano, —S(O)₂R11, C(O)NR8R9, hydroxymethyl,
R3 is hydrogen, methyl, NR15R16,
R4 is phenyl,
R5 is hydrogen,
R6 is hydrogen,
R8, R9 is hydrogen,
R11 is methyl
R13, R14 which can be the same or different, is hydrogen, methyl, ethyl, —CH(CH3)2, —(CH₂)₂—OCH₃, phenyl, —CH₂-(pyridyl),
R15, R16 which can be the same or different, is hydrogen, cyclopropyl, cyclobutyl which are optionally substituted by —C(O)OCH₂CH₃, cyclohexyl optionally substituted by hydroxy, or 1-4C-alkyl optionally substituted with methoxy, cyclopropyl, 4-fluoro-phenyl, or
R15, R16 together with the nitrogen atom to which they are attached form a morpholine ring,
X is (—CH₂—)ₙ,
n is 0, 1 or 2
Y is —CH₂—,
or an N-oxide, a salt, a tautomer or a stereoisomer of said compound, or a salt of said N-oxide, tautomer or stereoisomer.

6. The compound according to claim 1, which is selected from the group consisting of:
1-[4-(2-methyl-6-phenyl-[1,2,4]triazolo[1,5-a]pyrimidin-5-yl)phenyl]-cyclobutylamine,
1-[4-(2-methyl-6-phenyl-[1,2,4]triazolo[1,5-a]pyrimidin-5-yl)phenyl]-cyclopentylamine,
1-[4-(2-methyl-6-phenyl-[1,2,4]triazolo[1,5-a]pyrimidin-5-yl)phenyl]-cyclopropylamine,
1-[4-(2,7-dimethyl-6-phenyl-[1,2,4]triazolo[1,5-a]pyrimidin-5-yl)phenyl]-cyclobutylamine,
1-[4-(2-cyclopropyl-6-phenyl-[1,2,4]triazolo[1,5-a]pyrimidin-5-yl)phenyl]-cyclobutylamine,
1-[4-(6-Phenyl-imidazo[1,2-a]pyrimidin-7-yl)-phenyl]-cyclobutylamine,
1-[4-(2-Methyl-6-phenyl-imidazo[1,2-a]pyrimidin-7-yl)-phenyl]-cyclobutylamine,
{7-[4-(1-Amino-cyclobutyl)-phenyl]-6-phenyl-imidazo[1,2-a]pyrimidin-5-yl)-cyclopropylamine,
1-[4-(5-Morpholino-4-yl-6-phenyl-imidazo[1,2-a]pyrimidin-7-yl)-phenyl]-cyclobutylamine,
{7-[4-(1-Amino-cyclobutyl)-phenyl]-6-phenyl-imidazo[1,2-a]pyrimidin-5-yl}-((S)-2-methoxy-1-methyl-ethyl)-amine,
{7-[4-(1-Amino-cyclobutyl)-phenyl]-2-methyl-6-phenyl-imidazo[1,2-a]pyrimidin-5-yl)-cyclopropylamine,
{7-[4-(1-Amino-cyclobutyl)-phenyl]-2-methyl-6-phenyl-imidazo[1,2-a]pyrimidin-5-yl}-((S)-2-methoxy-1-methyl-ethyl)-amine,
{5-[4-(1-Aminocyclobutyl)-phenyl]-6-phenyl-2-pyridine-2-yl-[1,2,4]triazolo[1,5-a]pyrimidin-7-yl}-tert.-butylamine,
{5-[4-(1-Aminocyclobutyl)-phenyl]-6-phenyl-2-pyridine-2-yl-[1,2,4]triazolo[1,5-a]pyrimidin-7-yl}cyclopropylamine,
1-[4-(7-Morpholine-4-yl-6-phenyl-2-pyridine-2-yl-[1,2,4]triazolo[1,5-a]pyrimidin-5-yl)-phenyl]-cyclobutylamine, 1-{5-[4-(1-Aminocyclobutyl)-phenyl]-2-methyl-6-phenyl-[1,2,4]triazolo[1,5-a]pyrimidin-7-ylamino}-cyclobutanecarboxylic acid ethyl ester,
{5-[4-(1-Amino-cyclobutyl)-phenyl]-2-methyl-6-phenyl-[1,2,4]triazolo[1,5-a]pyrimidin-7-yl}-(3-methoxy-propyl)-amine,
{5-[4-(1-Amino-cyclobutyl)-phenyl]-2-methyl-6-phenyl-[1,2,4]triazolo[1,5-a]pyrimidin-7-yl}-[2-(4-fluoro-phenyl)-ethyl]-amine,
1-{4-[3-(4-Methansulfonyl-phenyl)-6-phenyl-imidazo[1,2-a]pyrimidin-7-yl]-phenyl}-cyclobutylamine,
{5-[4-(1-Amino-cyclobutyl)-phenyl]-6-phenyl-[1,2,4]triazolo[1,5-a]pyrimidin-2-yl}-(2-methoxy-ethyl)-amine,
{5-[4-(1-amino-cyclobutyl)-phenyl]-6-phenyl-[1,2,4]triazolo[1,5-a]pyrimidin-2-yl}-ethyl-amine,
1-{4-[2-(1-methyl-1H-pyrazol-4-yl)-6-phenyl-[1,2,4]triazolo[1,5-a]pyrimidin-5-yl]-phenyl}-cyclobutylamine,
{5-[4-(1-amino-cyclobutyl)-phenyl]-6-phenyl-[1,2,4]triazolo[1,5-a]pyrimidin-2-yl}-methyl-amine,
{5-[4-(1-Amino-cyclobutyl)-phenyl]-6-phenyl-[1,2,4]triazolo[1,5-a]pyrimidin-2-yl}-phenyl-amine,
{5-[4-(1-amino-cyclobutyl)-phenyl]-6-phenyl-[1,2,4]triazolo[1,5-a]pyrimidin-2-yl}-isopropyl-amine,
{5-[4-(1-amino-cyclobutyl)-phenyl]-6-phenyl-[1,2,4]triazolo[1,5-a]pyrimidin-2-yl}-pyridin-4-ylmethyl-amine,
{5-[4-(1-amino-cyclobutyl)-phenyl]-2-methyl-6-phenyl-[1,2,4]triazolo[1,5-a]pyrimidin-7-yl}-isopropyl-amine,
{5-[4-(1-amino-cyclobutyl)-phenyl]-2-methyl-6-phenyl-[1,2,4]triazolo[1,5-a]pyrimidin-7-yl}-cyclobutyl-amine,
{5-[4-(1-amino-cyclobutyl)-phenyl]-2-methyl-6-phenyl-[1,2,4]triazolo[1,5-a]pyrimidin-7-yl}-cyclopropyl methyl-amine,
{5-[4-(1-amino-cyclobutyl)-phenyl]-2-cyclopropyl-6-phenyl-[1,2,4]triazolo[1,5-a]pyrimidin-7-yl}-methyl-amine,
{5-[4-(1-amino-cyclobutyl)-phenyl]-2-methyl-6-phenyl-[1,2,4]triazolo[1,5-a]pyrimidin-7-yl}-ethyl-amine,
{5-[4-(1-amino-cyclobutyl)-phenyl]-2-methyl-6-phenyl-[1,2,4]triazolo[1,5-a]pyrimidin-7-yl}-cyclopropyl-amine,
{5-[4-(1-amino-cyclobutyl)-phenyl]-2-methyl-6-phenyl-[1,2,4]triazolo[1,5-a]pyrimidin-7-yl}-methyl-amine,
{5-[4-(1-amino-cyclobutyl)-phenyl]-2-methyl-6-phenyl-[1,2,4]triazolo[1,5-a]pyrimidin-7-yl}-(2-methoxy-ethyl)-amine,
4-{5-[4-(1-amino-cyclobutyl)-phenyl]-2-cyclopropyl-6-phenyl-[1,2,4]triazolo[1,5-a]pyrimidin-7-ylamino}cyclohexanol,
1-{4-[3-(4-Fluorophenyl)-6-phenyl-imidazo[1,2-a]pyrimidin-7-yl]-phenyl}-cyclobutylamine,
(5-{7-[4-(1-Amino-cyclobutyl)-phenyl]-6-phenyl-imidazo[1,2-a]pyrimidin-3-yl}-2-fluoro-phenyl)-methanol,
3-{7-[4-(1-Amino-cyclobutyl)-phenyl]-6-phenyl-imidazo[1,2-a]pyrimidin-3-yl}-benzamide,
1-{4-[6-Phenyl-3-(1H-pyrazol-4-yl)-imidazo[1,2-a]pyrimidin-7-yl]-phenyl}-cyclobutylamine.

7. A compound of formula (II), 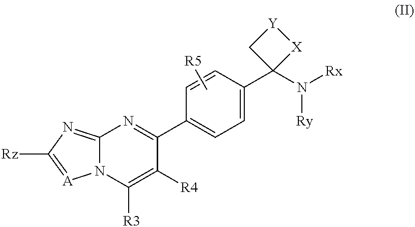
wherein
Rx is R6 or a protecting group
Ry is hydrogen or a protecting group,
whereby Rx and Ry together, or Y and Rx together, may form a cyclic protecting group,
Rz is R1 or R12
wherein
X is —(CH₂)ₙ—,
n is 0, 1, 2, or 3,
Y is —CH₂—, —CH(OH)—,
R1 is hydrogen, or a group selected from 1-6C-alkyl, 3-7C-cycloalkyl, aryl, heteroaryl,
wherein said group being optionally substituted, one or more times, identically or differently, with a substituent selected from:
hydroxy, halogen, 1-6C-alkyl, 1-4C-haloalkyl, 1-6C-alkoxy, —NR8R9, cyano, —C(O)NR8R9, —C(O)OR10, —NHC(O)R11, —NHS(O)₂R11,
R12 is hydrogen, halogen, —NR13R14, or a group selected from 1-6C-alkyl, 3-7C-cycloalkyl, aryl, heteroaryl,
wherein said group being optionally substituted, one or more times, identically or differently, with a substituent selected from:
hydroxy, halogen, 1-6C-alkyl, 1-4C-haloalkyl, 1-6C-alkoxy, —NR8R9, cyano, —C(O)NR8R9, —C(O)OR10, —NHC(O)R11, —NHS(O)₂R11,
R3 is hydrogen, 1-6C-alkyl, 3-7C-cycloalkyl, or NR15R16,
R4 is phenyl optionally substituted, one or more times, identically or differently, with a substituent selected from: 1-6C-alkyl, halogen, cyano,
R5 is hydrogen, halogen,
R6 is hydrogen, 1-6C-alkyl,
R8, R9 which can be the same or different, is
hydrogen,
1-4C-alkyl (optionally substituted in the same way or differently one or more times with halogen, hydroxy, mono- or di-(1-4C-alkylamino), 1-4C-alkoxy); or 3-7C-cycloalkyl,
or,
in the case of —NR8R9, R8 and R9 together with the nitrogen to which they are attached may also form a 3-6C-heterocyclic ring,
R10 is hydrogen, 1-6C-alkyl,
R11 is 1-4C-alkyl (optionally substituted in the same way of differently one or more times with halogen, hydroxy) or 3-7C-cycloalkyl,
R13, R14 which can be the same or different, is hydrogen, or a group selected from 1-6C-alkyl, 3-7C-cycloalkyl, aryl, heteroaryl, (1-6C-alkylen)-aryl, (1-6C-alkylen)-heteroaryl,
wherein said group being optionally substituted, one or more times, identically or differently, with a substituent selected from:
hydroxy, halogen, 1-6C-alkyl, 1-4C-haloalkyl, 1-6C-alkoxy, cyano,
R15, R16 which can be the same or different, is hydrogen, or a group selected from 1-6C-alkyl, 3-7C-cycloalkyl, (1-6C-alkylen)-aryl, (1-6C-alkylen)-heteroaryl,
wherein said group being optionally substituted, one or more times, identically or differently, with a substituent selected from:
hydroxy, halogen, 1-6C-alkyl, 1-4C-haloalkyl, 1-6C-alkoxy, cyano, 3-7-cycloalkyl, heterocyclyl, —C(O)NR10R11, —C(O)OR10, —NHC(O)R11, —NHS(O)₂R11,
or,
R15 and R16 together with the nitrogen to which they are attached may also form a 3-6C-heterocyclic ring.

8. (canceled)

9. (canceled)

10. A method for the treatment or prophylaxis of a hyperproliferative disease or disorders responsive to induction of apoptosis comprising administering to a human in need thereof an effective amount of a compound according to claim 1.

11. The method according to claim 10, wherein the hyperproliferative disease or disorder responsive to induction of apoptosis is a benign or malignant neoplasm.

12. A pharmaceutical composition comprising at least one compound according to claim 1, together with at least one pharmaceutically acceptable auxiliary.

13. (canceled)

14. A pharmaceutical combination comprising at least one compound according to claim 1, and one or more second active ingredients selected from chemotherapeutic anti-cancer agents and target-specific anti-cancer agents.

15. (canceled)

16. The compound according to claim 2,
wherein
R1 is hydrogen, or a group selected from 1-6C-alkyl, 3-7C-cycloalkyl, aryl, heteroaryl,
wherein said group being optionally substituted, one or more times, identically or differently, with a substituent selected from:
hydroxy, halogen, 1-6C-alkyl, 1-4C-haloalkyl, 1-6C-alkoxy, —NR8R9, cyano, —C(O)NR8R9, —C(O)OR10, —NHC(O)R11, —NHS(O)₂R11,
R12 is hydrogen, halogen, —NR13R14, or a group selected from 1-6C-alkyl, 3-7C-cycloalkyl, aryl, heteroaryl,
wherein said group being optionally substituted, one or more times, identically or differently, with a substituent selected from:
hydroxy, halogen, 1-6C-alkyl, 1-4C-haloalkyl, 1-6C-alkoxy, —NR8R9, cyano, —C(O)NR8R9, —C(O)OR10, —NHC(O)R11, —NHS(O)₂R11,
R2 is hydrogen, halogen, cyano, or a group selected from 1-6C-alkyl, 3-7C-cycloalkyl, aryl, heteroaryl, (1-6C-alkylen)-aryl, (1-6C-alkylen)-heteroaryl,
wherein said group being optionally substituted, one or more times, identically or differently, with a substituent selected from:
hydroxy, halogen, 1-6C-alkyl, 1-4C-haloalkyl, 1-6C-hydroxyalkyl, 1-6C-alkoxy, —NR8R9, cyano, —C(O)NR8R9, —C(O)OR10, —NHC(O)R11, —NHS(O)₂R11, —S(O)₂R11, —S(O)₂NR8R9,
R3 is hydrogen, 1-6C-alkyl, 3-7C-cycloalkyl, or NR15R16,
R4 is phenyl,
R5 is hydrogen,
R6 is hydrogen,
R8, R9 which can be the same or different, is hydrogen, 1-4C-alkyl (optionally substituted in the same way or differently one or more times with halogen,
hydroxy, mono- or di-(1-4C-alkylamino), 1-4C-alkoxy); or 3-7C-cycloalkyl,
or,
in the case of —NR8R9, R8 and R9 together with the nitrogen to which they are attached may also form a 3-6C-heterocyclic ring,
R10 is hydrogen, 1-6C-alkyl,
R11 is 1-4C-alkyl (optionally substituted in the same way of differently one or more times with halogen, hydroxy) or 3-7C-cycloalkyl,
R13, R14 which can be the same or different, is hydrogen, or a group selected from 1-6C-alkyl, 3-7C-cycloalkyl, aryl, heteroaryl, (1-6C-alkylen)-aryl, (1-6C-alkylen)-heteroaryl,
wherein said group being optionally substituted, one or more times, identically or differently, with a substituent selected from:
hydroxy, halogen, 1-6C-alkyl, 1-4C-haloalkyl, 1-6C-alkoxy, cyano,
R15, R16 which can be the same or different, is hydrogen, or a group selected from 1-6C-alkyl, 3-7C-cycloalkyl, (1-6C-alkylen)-aryl, (1-6C-alkylen)-heteroaryl,
wherein said group being optionally substituted, one or more times, identically or differently, with a substituent selected from:
hydroxy, halogen, 1-6C-alkyl, 1-4C-haloalkyl, 1-6C-alkoxy, cyano, 3-7-cycloalkyl, heterocyclyl, —C(O)OR10, or
or R15, R16 together with the nitrogen atom to which they are attached may also form a 5- or 6-membered heterocyclic ring optionally containing an additional nitrogen- or oxygen atom,
X is —(CH₂)ₙ—,
n is 0, 1 or 2,
Y is —CH₂—, —CH(OH)—,
or an N-oxide, a salt, a tautomer or a stereoisomer of said compound, or a salt of said N-oxide, tautomer or stereoisomer.

17. The compound according to claim 2,
wherein
R1 is hydrogen, 1-6C-alkyl,
R12 is hydrogen, 1-6C-alkyl, 3-7C-cycloalkyl, heteroaryl, NR13R14,
R2 is hydrogen, aryl, heteroaryl
wherein said aryl being optionally substituted, one or more times, identically or differently, with a substituent selected from:
halogen, 1-6C-hydroxyalkyl, cyano, —S(O)₂R11, C(O)NR8R9,
R3 is hydrogen, 1-6C-alkyl, NR15R16,
R4 is phenyl,
R5 is hydrogen,
R6 is hydrogen,
R8, R9 is hydrogen,
R10 is hydrogen, 1-4C-alkyl,
R11 is 1-4C-alkyl,
R13, R14 which can be the same or different, is hydrogen, or a group selected from 1-6C-alkyl, aryl, (1-6C-alkylen)-heteroaryl, wherein said group is optionally substituted with 1-6C-alkoxy,
R15, R16 which can be the same or different, is hydrogen, or a group selected from 1-6C-alkyl, 3-7C-cycloalkyl, (1-6C-alkylen)-aryl,
wherein said group being optionally substituted, one or more times, identically or differently, with a substituent selected from:
hydroxy, halogen, 1-6C-alkoxy, 3-7C-cycloalkyl, —C(O)OR10,
or R15, R16 together with the nitrogen atom to which they are attached may also form a 6-membered ring containing one oxygen atom,
X is —(CH₂)ₙ—,
n is 0, 1 or 2,
Y is —CH₂—,
or an N-oxide, a salt, a tautomer or a stereoisomer of said compound, or a salt of said N-oxide, tautomer or stereoisomer.

18. The compound according to claim 2,
wherein
R1 is hydrogen, methyl,
R12 is hydrogen, methyl, cyclopropyl, N-methyl-pyrazolyl, pyridyl, NR13R14,
R2 is hydrogen, 1H-pyrazol-yl, or phenyl substituted one or more times with fluorine, cyano, —S(O)₂R11, C(O)NR8R9, hydroxymethyl,
R3 is hydrogen, methyl, NR15R16,
R4 is phenyl,
R5 is hydrogen,
R6 is hydrogen,
R8, R9 is hydrogen,
R11 is methyl
R13, R14 which can be the same or different, is hydrogen, methyl, ethyl, —CH(CH3)2, —(CH₂)₂—OCH₃, phenyl, —CH₂-(pyridyl),
R15, R16 which can be the same or different, is hydrogen, cyclopropyl, cyclobutyl which are optionally substituted by —C(O)OCH₂CH₃, cyclohexyl optionally substituted by hydroxy, or 1-4C-alkyl optionally substituted with methoxy, cyclopropyl, 4-fluoro-phenyl, or
R15, R16 together with the nitrogen atom to which they are attached form a morpholine ring,
X is (—CH₂—)ₙ,
n is 0, 1 or 2
Y is —CH₂—,
or an N-oxide, a salt, a tautomer or a stereoisomer of said compound, or a salt of said N-oxide, tautomer or stereoisomer.
